(19) United States
(12) Patent Application Publication
(10) Pub. No.: US 2011/0172296 A1
(43) Pub. Date: Jul. 14, 2011

(54) MODULATION OF TRANSFORMING GROWTH FACTOR-BETA 1 EXPRESSION

(76) Inventors: C. Frank Bennett, Carlsbad, CA (US); Susan M. Freier, San Diego, CA (US); Nicholas M. Dean, Olivenhain, CA (US); J. Gordon Foulkes, Encinitas, CA (US); William A. Gaarde, Carlsbad, CA (US)

(21) Appl. No.: 13/005,508

(22) Filed: Jan. 12, 2011

Related U.S. Application Data
(60) Provisional application No. 61/294,303, filed on Jan. 12, 2010.

(51) Int. Cl. A61K 31/7088; C07H 21/04; C07H 21/02; A61P 17/02

(52) U.S. Cl. 514/44 R; 536/23.1; 536/24.5

(57) ABSTRACT

Provided are compounds capable of inhibiting expression of TGF-beta 1 and compositions containing same as well as methods using such compounds for treating fibrotic diseases including the reduction of scarring resulting from wound healing.

### RELATED APPLICATIONS

This application claims priority under 35 USC 119(e) to Provisional Patent Application Ser. No. 61/294,303, filed Jan. 12, 2010, which is incorporated herein by reference in its entirety.

### FIELD OF THE INVENTION

This invention concerns methods, compounds, and compositions for modulating expression of TGF-beta1 to treat, prevent, or ameliorate TGF-beta1 associated diseases and disorders.

### SEQUENCE LISTING

The present application is being filed along with a Sequence Listing in electronic format. The Sequence Listing is provided as a file entitled BIOL0118USSEQ.txt, created Jan. 12, 2011, which is 92 Kb in size. The information in the electronic format of the sequence listing is incorporated herein by reference in its entirety.

### BACKGROUND

Fibrosis is a pathological process that generally results from injury and can occur in any organ. Fibrosis is the excessive accumulation of extracellular matrix within a tissue, forming scar tissue. Such accumulation can cause dysfunction and, potentially, organ failure. Fibrosis can be either chronic or acute. Chronic fibrosis includes fibrosis of the major organs, most commonly liver, lung, kidney and/or heart, and normally has a genetic or idiopathic origin. Progressive fibrosis of the kidney is the main cause of chronic renal disease. In diabetics, fibrosis within glomeruli (glomerulosclerosis) and between tubules (tubulointerstitial fibrosis) causes the progressive loss of renal function that leads to end-stage renal disease. Fibrotic lung disorders can result in severe impairment of lung function.

Another form of fibrosis occurs in the skin, commonly referred to as scarring, which from an evolutionary perspective can be viewed as a natural part of the healing process. Skin scars occur when the dermis is damaged. Abnormal scarring can result from the overproduction of collagen, which causes the scar to be raised above the surrounding skin. Hypertrophic scars take the form of a red raised lump on the skin, but generally do not grow beyond the boundaries of the original wound. Keloid scars are a more serious, disfiguring form of scarring, potentially growing indefinitely into large, benign tumor-like growths. Keloid scars can be caused by surgery, an accident, acne or, sometimes, body piercings. In some people, keloid scars can form spontaneously. Keloid scars are often found in individuals of darker complexion.

Acute fibrosis is associated with injury, often as a result of surgery. Surgical adhesion represents the largest class of acute fibrosis. Surgery often results in excessive scarring and fibrous adhesions. It is estimated that over 90% of post-surgical patients are affected by adhesions. Abdominal adhesions can lead to small bowel obstruction and female infertility. Fibrosis after neck and back surgery (laminectomy, discectomy) can cause significant pain. Fibrosis after eye surgery can impair vision. Pericardial adhesions after coronary bypass surgery, fibrosis after organ transplant rejection and general scarring after plastic surgery are other examples of acute fibrosis.

Reduction or prevention of fibrosis represents a major unmet medical need. There is currently a lack of acceptable options for treating almost any fibrotic condition. Thus, the identification of genes which are involved in this process and the development of drugs targeting such genes remains a key unmet clinical goal. It is therefore an object herein to provide compounds and methods for the treatment of such diseases and disorders.

Discovered as a growth factor (Growth Factors 8 (1993), pp. 1-9; Proc. Natl. Acad. Sci. USA 82 (1985), pp. 119-123), transforming growth factor-beta (TGF-β) has emerged as a pivotal immunoregulatory cytokine (J. Exp. M 180 (1994), pp. 1587-1590; Int. Rev. Immunol. 16 (1998), pp. 553-580; Annu. Rev. Immunol. 16 (1998), pp. 137-161) which regulates biological processes such as cell proliferation, differentiation and immune reaction (J. Cell. Biochem. (2007), pp. 593-608). Among its many functions, it has been implicated in tissue repair by stimulating the deposition of extracellular matrix in multiple ways. TGF-β stimulates the synthesis of matrix proteins, including fibronectin, collagens and proteoglycans. It also blocks the degradation of matrix by inhibiting protease secretion and by inducing the expression of protease inhibitors. It facilitates cell-matrix adhesion and matrix deposition via modulation of expression of integrin matrix receptors. TGF-β also upregulates its own expression. Of the multiple isoforms, TGF-β1, 2, and 3 have been identified in mammalian species and have demonstrated overlapping and distinct functional properties (J. Cell. Biochem. (2007), pp. 593-608).

There is a currently a lack of acceptable options for treating conditions of scarring and fibrosis. It is therefore an object herein to provide compounds and methods for the treatment of such diseases and disorder.

Antisense technology is emerging as an effective means for reducing the expression of certain gene products and may therefore prove to be uniquely useful in a number of therapeutic, diagnostic, and research applications for the modulation of TGF-beta1. Certain TGF-beta1 targeting antisense oligonucleotides (ASOs) have been described in U.S. Pat. Nos. 5,683,988, 6,436,909; 6,455,689 and 6,972,171. However, there remains a need for additional such compounds, particularly compounds with improved characteristics, such as having increased potency and/or reduced toxicity compared to those previously described. It is an object herein to provide additional compounds and methods including, for example, compounds and methods demonstrating improved characteristics such as, but not limited to, improved potency and/or improved tolerability.

### FIGURES

FIG. 1: A chart showing the effect of antisense inhibition on skin thickening compared to the control at day 18 after bleomycin treatment as described in Example 10.

FIG. 2: A chart showing the effect of antisense inhibition on skin breaking tension compared to the control at day 18 after bleomycin treatment as described in Example 10.

### SUMMARY

Provided herein are methods, compounds, and compositions for modulating of TGF-beta1. In certain embodiments, TGF-beta1 specific inhibitors are provided which modulate expression of TGF-beta1. In certain embodiments, TGF-beta1 specific inhibitors are nucleic acids, antisense compounds or antisense oligonucleotides. Pharmaceutical and other compositions comprising the TGF-beta1 specific inhibitors are also provided.

Further provided are methods of modulating TGF-beta1 in cells or tissues, comprising contacting said cells or tissues with one or more of the TGF-beta1 specific inhibitors or compositions. Further provided are methods of treating an animal, particularly a human, suspected of having or being prone to a disease or condition associated with expression of TGF-beta1 by administering a therapeutically or prophylactically effective amount of one or more of the TGF-beta1 specific inhibitors or compositions provided herein. In certain embodiments, modulation of TGF-beta1 can be measured by mRNA and/or protein expression levels.

Further provided are TGF-beta1 specific inhibitors or compositions having superior inhibitory activity compared to previously described TGF-beta1 targeting antisense oligonucleotides. Also provided are unique TGF-beta1 mRNA sequence ‘hot-spots”, the target of which with TGF-beta1 specific inhibitors or compositions results in superior reduction of TGF-beta1 expression. Also provided are TGF-beta1 specific inhibitors or compositions with superior tolerability characteristics.

### DETAILED DESCRIPTION OF THE INVENTION

It is to be understood that both the foregoing general description and the following detailed description are exemplary and explanatory only and are not restrictive of the invention which is defined by the claims. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the inventions described.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. To the extent permitted, all patents, applications, published applications and other publications, GENBANK Accession Numbers and associated sequence information obtainable through databases such as National Center for Biotechnology Information (NCBI) and other data referred to herein are hereby incorporated by reference in their entirety.

Unless otherwise indicated, the following terms have the following meanings:

“2′-O-methoxyethyl” (also 2′-MOE, 2′-O-(2-methoxyethyl) and 2′-O(CH₂)₂—OCH₃) refers to an O-methoxy-ethyl modification of the 2′ position of a furosyl ring. A 2′-β-methoxyethyl modified sugar is a modified sugar.

“2′-O-methoxyethyl nucleoside” means a nucleoside comprising a 2′-O-methoxyethyl modified sugar moiety.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5′ position. A 5-methylcytosine is a modified nucleobase.

“About” means within ±10% of a value. For example, if it is stated, “the LDL levels of nave mice are about 40 mg/dL”, it is implied that the LDL levels are within a range of 36 mg/dL and 44 mg/dL. “Administered concomitantly” refers to the co-administration of two agents in any manner in which the pharmacological effects of both are manifest in the patient. Concomitant administration does not require that both agents be administered in a single pharmaceutical composition, in the same dosage form, at the same time or by the same route of administration.

“Administering” means providing a pharmaceutical agent to an individual, and includes, but is not limited to, administering by a medical professional and self-administering.

“Ameliorate” means to make better or improve the symptoms of a condition or disease in a subject.

“Animal” refers to human or non-human animals, including, but not limited to, mice, rats, rabbits, dogs, cats, pigs, horses and non-human primates, including, but not limited to, monkeys and chimpanzees.

“Antisense compound” means an oligomeric compound that is capable of undergoing hybridization to a target nucleic acid through hydrogen bonding.

“Antisense inhibition” means the reduction of target nucleic acid or protein levels in the presence of an antisense compound complementary to a target nucleic acid compared to the target nucleic acid or protein levels in the absence of the antisense compound.

“Antisense oligonucleotide” means a single-stranded oligonucleotide having a nucleobase sequence that permits hybridization to a complementary region or segment of a target nucleic acid.

“Bicyclic sugar” means a furosyl ring modified by the bridging of two non-geminal ring atoms. A bicyclic sugar is a modified sugar moiety.

“Cap structure” or “terminal cap moiety” means a chemical modification, which has been incorporated at a terminus of an antisense compound. An antisense compound can have both termini “capped”.

“Chimeric antisense compounds” means antisense compounds that have at least 2 chemically distinct regions, each region can include a plurality of subunits.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses administration in parallel or sequentially.

“Complementarity” means the capacity for pairing between nucleobases of a first nucleic acid and a second nucleic acid. In certain embodiments, complementarity between the first and second nucleic acid may be between two DNA strands, between two RNA strands, or between a DNA and an RNA strand. In certain embodiments, some of the nucleobases on one strand are matched to a complementary hydrogen bonding base on the other strand. In certain embodiments, all of the nucleobases on one strand are matched to a complementary hydrogen bonding base on the other strand. In certain embodiments, a first nucleic acid is an antisense compound and a second nucleic acid is a target nucleic acid. In certain such embodiments, an antisense oligonucleotide is a first nucleic acid and a target nucleic acid is a second nucleic acid.

“Comprise,” “comprises” and “comprising” are to be understood to imply the inclusion of a stated step or element or group of steps or elements but not the exclusion of any other step or element or group of steps or elements.

“Contiguous nucleobases” means nucleobases immediately adjacent to each other.

“Cross-reactive” means an oligomeric compound targeting one nucleic acid sequence can hybridize to a different nucleic acid sequence. For example, in some instances an antisense oligonucleotide targeting human TGF-beta1 can cross-react with a murine TGF-beta1. Whether an oligomeric compound cross-reacts with a nucleic acid sequence other than its designated target depends on the degree of complementarity the compound has with the non-target nucleic acid sequence. The higher the complementarity between the oligomeric compound and the non-target nucleic acid, the more likely the oligomeric compound will cross-react with the nucleic acid.

“Cure” means a method that restores health or a prescribed treatment for an illness.

“Deoxyribonucleotide” means a nucleotide having a hydrogen atom at the 2′ position of the sugar portion of the nucleotide. Deoxyribonucleotides can be modified with any of a variety of substituents.

“Designing” or “Designed to” refer to the process of designing an oligomeric compound that specifically hybridizes with a selected nucleic acid molecule or portion thereof.

“Diluent” means an ingredient in a composition that lacks pharmacological activity, but is pharmaceutically necessary or desirable. For example, in drugs that are injected, the diluent can be a liquid, e.g. saline solution.

“Dose” means a specified quantity of a pharmaceutical agent provided in a single administration, or in a specified time period. In certain embodiments, a dose can be administered in two or more boluses, tablets, or injections. For example, in certain embodiments, where subcutaneous administration is desired, the desired dose requires a volume not easily accommodated by a single injection. In such embodiments, two or more injections can be used to achieve the desired dose. In certain embodiments, a dose can be administered in two or more injections to minimize injection site reaction in an individual. In other embodiments, the pharmaceutical agent is administered by infusion over an extended period of time or continuously. Doses can be stated as the amount of pharmaceutical agent per hour, day, week or month. Doses can be expressed as mg/kg or g/kg.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Duration” means the period of time during which an activity or event continues. In certain embodiments, the duration of treatment is the period of time during which doses of a pharmaceutical agent are administered.

“Efficacy” means the ability to produce a desired effect.

“Expression” includes all the functions by which a gene's coded information is converted into structures present and operating in a cell. Such structures include, but are not limited to, the products of transcription and translation.

“First agent” or “first therapeutic agent” means an agent that can be used in combination with a “second agent”. In certain embodiments, the first agent is any antisense compound, oligonucleotide or composition that inhibits TGF-beta1 described herein.

“Fully complementary” or “100% complementary” means each nucleobase of a first nucleic acid has a complementary nucleobase in a second nucleic acid. In certain embodiments, a first nucleic acid is an antisense compound and a second nucleic acid is a target nucleic acid. In certain such embodiments, an antisense oligonucleotide is a first nucleic acid and a target nucleic acid is a second nucleic acid.

“Gapmer” means an antisense compound in which an internal position having a plurality of nucleotides that supports RNaseH cleavage is positioned between external regions having one or more nucleotides that are chemically distinct from the nucleosides of the internal region. A “gap segment” means the plurality of nucleotides that make up the internal region of a gapmer. A “wing segment” can be the external region of a gapmer.

“Gap-widened” means an antisense compound has a gap segment of 12 or more contiguous 2′-deoxyribonucleotides positioned between and immediately adjacent to 5′ and 3′ wing segments of from one to six nucleotides having modified sugar moieties.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Immediately adjacent” means there are no intervening nucleotides between the immediately adjacent elements. For example, between regions, segments, nucleotides and/or nucleosides.

“Induce”, “inhibit”, “potentiate”, “elevate”, “increase”, “decrease” or the like, e.g., denote quantitative differences between two states. For example, “an amount effective to inhibit the activity or expression of TGF-beta1” means that the level of activity or expression of TGF-beta1 in a treated sample will differ from the level of TGF-beta1 activity or expression in untreated cells. Such terms are applied to, for example, levels of expression, and levels of activity.

“Inhibiting the expression or activity” refers to a reduction, blockade of the expression or activity of the target and does not necessarily indicate a total elimination of expression or activity.

“Internucleoside linkage” refers to the chemical bond between nucleosides.

“Intravenous administration” means administration into a vein.

“Linked nucleosides” means adjacent nucleosides which are bonded together.

“Mismatch” refers to a non-complementary nucleobase within an oligomeric compound complementary to a target nucleic acid.

“Modified internucleoside linkage” refers to a substitution and/or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases, adenine (A) and guanine (G), and the pyrimidine bases, thymine (T), cytosine (C) and uracil (U).

“Modified oligonucleotide” means an oligonucleotide comprising a modified internucleoside linkage, a modified sugar, and/or a modified nucleobase. A modified oligonucleotide can also have a nucleoside mimetic or nucleotide mimetic.

“Modified sugar” refers to a substitution and/or any change from a natural sugar.

“Modulation” means a perturbation of function, for example, one associated with either an increase (stimulation or induction) or a decrease (inhibition or reduction) in expression.

“Monomer” refers to a single unit of an oligomer. Monomers include, but are not limited to, nucleosides and nucleotides, whether naturally occurring or modified.

“Motif” means the pattern of unmodified and modified nucleosides in an antisense compound.

“Naturally occurring internucleoside linkage” means a 3′ to 5′ phosphodiester linkage.

“Natural sugar” means a sugar found in DNA (2′-H) or RNA (2′-OH).

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, a complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the oligonucleotide and the target nucleic acid are considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleotide” means a nucleoside having a phosphate group covalently linked to the sugar portion of the nucleoside.

“Nucleoside mimetic” includes those structures used to replace the sugar or the sugar and the base, and not necessarily the linkage at one or more positions of an oligomeric compound; for example, nucleoside mimetics having morpholino, cyclohexenyl, cyclohexyl, tetrahydropyranyl, bicyclo or tricyclo sugar mimetics, such as non furanose sugar units.

“Nucleotide mimetic” includes those structures used to replace the nucleoside and the linkage at one or more positions of an oligomeric compound; for example, peptide nucleic acids or morpholinos (morpholinos linked by —N(H)—C(═O)—O— or other non-phosphodiester linkage).

“Oligomeric compound” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration,” means administration by a manner other than through the digestive tract e.g., through topical administration, injection or infusion. Parenteral administration includes, but is not limited to, subcutaneous administration, intravenous administration, and intramuscular administration.

“Pharmaceutically acceptable carrier” or “Pharmaceutically acceptable diluent” means a carrier or diluent that does not interfere with the structure or function of the oligonucleotide. Certain of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

“Pharmaceutically acceptable salts” or “salts” means physiologically and pharmaceutically acceptable salts of antisense compounds, i.e., salts that retain the desired biological activity of the parent oligonucleotide and do not impart undesired toxicological effects thereto.

“Pharmaceutical composition” or “composition” means a mixture of substances suitable for administering to an animal. For example, a composition can comprise one or more antisense oligonucleotides and a sterile aqueous solution.

“Phosphorothioate internucleoside linkage” or “phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevention” or “preventing” refers to delaying or forestalling the onset or development of a condition or disease for a period of time from hours to days, preferably weeks to months to years or permanently.

“Prodrug” means a therapeutic agent that is prepared in an inactive form that is converted to an active form (i.e., a drug) within the body or cells thereof by the action of endogenous or non-endogenous enzymes or other chemicals and/or conditions.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides can be modified with any of a variety of substituents.

“Second agent” or “second therapeutic agent” means an agent that can be used in combination with a “first agent”. A second therapeutic agent can be any agent that inhibits or prevents excess collagen production. A second therapeutic agent can include, but is not limited to, an siRNA or antisense oligonucleotide, including antisense oligonucleotides targeting TGF-beta1. A second agent can also include anti-TGF-beta antibodies, TGF-beta receptor inhibitors, factors that modulate connective tissue growth factor (CTGF) (e.g., an siRNA or antisense oligonucleotide), or non-specific agents, such as steroids. A second therapeutic agent can also include, but is not limited to, silicone wrap, TGF-β3 (e.g. Juvista), 17β-estrodiol (e.g. Zesteem), IL-10 (e.g. Prevascar), mannose 6-phosphate (e.g. Juvidex), AZX100 (a 24 amino acid peptide developed by Capstone Therapeutics), serum amyloid protein, or antibodies targeting integrin αvβ6, or molecules that inhibit the activity of ALK-4 and/or ALK-5 (i.e. the TGF-beta receptors), Dermagraft, Apligraf, Regranex (PDGF), electrical stimulation, “growth factors” as a category, dressings as a category, small intestinal submucosa, (SIS), Promogran, or hyperbaric oxygen.

“Segments” are defined as smaller, sub-portions of regions within a nucleic acid. For example, a “target segment” means the sequence of nucleotides of a target nucleic acid to which one or more antisense compounds is targeted. “5′ target site” refers to the 5′-most nucleotide of a target segment. “3′ target site” refers to the 3′-most nucleotide of a target segment.

“Shortened” or “truncated” versions of antisense oligonucleotides or target nucleic acids taught herein have one, two or more nucleosides deleted.

“Side effects” mean physiological responses attributable to a treatment other than the desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand. “Single-stranded modified oligonucleotide” means a modified oligonucleotide which is not hybridized to a complementary strand.

“siRNA” is defined as a double-stranded compound having a first and second strand and comprises a central complementary portion between said first and second strands and terminal portions that are optionally complementary between said first and second strands or with a target mRNA. In one non-limiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand can contain modifications or additions to either terminus.

“Sites,” as used herein, are defined as unique nucleobase positions within a target nucleic acid.

“Slows progression” means a decrease in the development of a disease, condition or symptom.

“Specifically hybridizable” means an antisense compound that hybridizes to a target nucleic acid to induce a desired effect, while exhibiting minimal or no effects on non-target nucleic acids.

“Subcutaneous administration” means administration just below the skin.

“Subject” means a human or non-human animal selected for treatment or therapy.

“Targeted” or “targeted to” means having a nucleobase sequence that will allow specific hybridization of an antisense compound to a target nucleic acid to induce a desired effect.

“Target nucleic acid,” “target RNA,” “target RNA transcript” and “nucleic acid target” all mean a nucleic acid capable of being targeted by antisense compounds.

“Targeting” means the process of design and selection of an antisense compound that will specifically hybridize to a target nucleic acid and induce a desired effect.

“TGF-beta1” means any nucleic acid or protein sequence encoding TGF-beta1. For example, in certain embodiments, TGF-beta1 includes a DNA sequence encoding TGF-beta1, an RNA sequence transcribed from DNA encoding TGF-beta1 (including genomic DNA comprising introns and exons), an mRNA sequence encoding TGF-beta1, or a peptide sequence encoding TGF-beta1.

“TGF-beta1 nucleic acid” means any nucleic acid encoding TGF-beta1. For example, in certain embodiments, a TGF-beta1 nucleic acid includes, without limitation, a DNA sequence encoding TGF-beta1, an RNA sequence transcribed from DNA encoding TGF-beta1, and an mRNA sequence encoding TGF-beta1.

“TGF-beta1 mRNA” means an mRNA encoding a TGF-beta1 protein.

“Therapeutically effective amount” or “effective amount” means an amount of a pharmaceutical agent such as an antisense compound that provides a therapeutic benefit to an individual. “Effective amount” in the context of modulating an activity or of treating or preventing a condition means the administration of that amount of active ingredient or pharmaceutical agent such as an antisense compound to a subject in need of such modulation, such as inhibition, treatment or prophylaxis, either in a single dose or as part of a series of doses, that is effective for modulating that activity, such as inhibition of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Treatment” refers to administering a composition of the invention to effect an alteration or improvement of a disease, condition or symptom.

“Unmodified nucleotide” means a nucleotide composed of naturally occurring nucleobases, sugar moieties and internucleoside linkages. In certain embodiments, an unmodified nucleotide is a RNA nucleotide (i.e., β-D-ribonucleosides) or a DNA nucleotide (i.e., β-D-deoxyribonucleoside).

“Wing segment” means one or a plurality of nucleosides modified to impart to an oligonucleotide properties such as enhanced inhibitory activity, increased binding affinity for a target nucleic acid, or resistance to degradation by in vivo nucleases.

### CERTAIN EMBODIMENTS

Provided herein are methods, compounds, and compositions for modulating TGF-beta1 activity level or expression.

In certain embodiments, TGF-beta1 specific inhibitors are provided for reduction of TGF-beta1. In certain embodiments, TGF-beta1 specific inhibitors are nucleic acids, antisense compounds, or antisense oligonucleotides. In certain embodiments, an antisense compound includes an antisense oligonucleotide.

In certain embodiments, the TGF-beta1 specific inhibitors are targeted to a TGF-beta1 nucleic acid. In certain embodiments, the TGF-beta1 nucleic acid is a human TGF-beta1 nucleic acid with any of the sequences set forth in GENBANK Accession No. NM_{—}000660.3 (incorporated herein as SEQ ID NO: 1) and GENBANK Accession No. NT 011109.15 truncated from 14103000 to 1413000, (incorporated herein as SEQ ID NO: 2). In certain embodiments, the TGF-beta1 nucleic acid is a murine TGF-beta1 nucleic acid with the sequence set forth in GENBANK Accession No. NT 039413.7 truncated at nucleotides 23471000 to 23492000 (incorporated herein as SEQ ID NO: 3).

In certain embodiments, the compounds or oligonucleotides provided herein have 12 to 30 linked nucleosides and have a nucleobase sequence comprising a contiguous nucleobase portion of a nucleobase sequence selected from among the nucleobase sequences recited in SEQ ID NOs: 4-159. In certain embodiments, the portion is at least 8, 9, 10, 11, 12, 13, 14, 15, 16, 17, 18, 19 or 20 contiguous nucleobases of a nucleobase sequence selected from among the nucleobase sequences recited in SEQ ID NOs: 4-159.

In certain embodiments, an antisense compound or oligonucleotide targeted to a TGF-beta1 nucleic acid is 20 subunits in length. In such embodiments, antisense compounds or oligonucleotides are 20 linked subunits in length.

In certain embodiments, an antisense compound or oligonucleotide targeted to a TGF-beta1 nucleic acid is 20 nucleobases in length. In certain such embodiments, an antisense compound or oligonucleotide targeted to a TGF-beta1 nucleic acid is 20 linked nucleobases in length.

In certain embodiments, antisense compounds or oligonucleotides target a range of a TGF-beta1 nucleic acid. In certain embodiments, such compounds or oligonucleotides targeted to a range of a TGF-beta1 nucleic acid have at least an 8 nucleobase portion that is complementary to an equal length portion within the range. In certain embodiments, such compounds or oligonucleotides, which are targeted to a range of a TGF-beta1 nucleic acid, have at least an 8 nucleobase portion that is complementary to an equal length portion within the range or target region identified herein.

In certain embodiments, an antisense compound or oligonucleotide targeted to a TGF-beta1 nucleic acid target the following nucleotide regions of SEQ ID NO: 1:1-22, 1-20, 140-179, 159-179, 236-255, 280-327, 282-363, 282-305, 290-363, 290-327, 292-321, 371-400, 373-400, 375-396, 381-400, 446-497, 446-495, 446-465, 538-676, 538-640, 558-640, 625-676, 627-676, 629-668, 631-652, 637-664, 1139-1207, 1149-1170, 1139-1170, 2109-2203, 2109-2192, 2109-2176, 2109-2138, 2111-2176, 2111-2138, 2111-2136, 2111-2192, 2157-2203, or 2157-2192.

In certain embodiments, an antisense compound or oligonucleotide targeted to a TGF-beta1 nucleic acid hybridizes exclusively within the following nucleotide regions of SEQ ID NO: 1:1-22, 1-20, 140-179, 159-179, 236-255, 280-327, 282-363, 282-305, 290-363, 290-327, 292-321, 371-400, 373-400, 375-396, 381-400, 446-497, 446-495, 446-465, 538-676, 538-640, 558-640, 625-676, 627-676, 629-668, 631-652, 637-664, 1139-1207, 1149-1170, 1139-1170, 2109-2203, 2109-2192, 2109-2176, 2109-2138, 2111-2176, 2111-2138, 2111-2136, 2111-2192, 2157-2203, or 2157-2192.

In certain embodiments, antisense compounds or oligonucleotides target a region of a TGF-beta1 nucleic acid. In certain embodiments, such compounds or oligonucleotides targeted to a region of a TGF-beta1 nucleic acid have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobase portion complementary to an equal length portion of a region recited herein. For example, the portion can consist of an 8 contiguous nucleobase portion complementary to an equal length portion of a region recited herein. In certain embodiments, such compounds or oligonucleotides target the following nucleotide regions of SEQ ID NO: 1:1-22, 1-20, 140-179, 159-179, 236-255, 280-327, 282-363, 282-305, 290-363, 290-327, 292-321, 371-400, 373-400, 375-396, 381-400, 446-497, 446-495, 446-465, 538-676, 538-640, 558-640, 625-676, 627-676, 629-668, 631-652, 637-664, 1139-1207, 1149-1170, 1139-1170, 2109-2203, 2109-2192, 2109-2176, 2109-2138, 2111-2176, 2111-2138, 2111-2136, 2111-2192, 2157-2203, or 2157-2192.

In certain embodiments, the following nucleotide regions of SEQ ID NO: 1, when targeted by antisense compounds or oligonucleotides, display at least 60% inhibition: 1-20, 159-255, 282-305, 290-363, 375-396, 381-465, 538-676, or 1139-2308.

In certain embodiments, the following nucleotide regions of SEQ ID NO: 1, when targeted by antisense compounds or oligonucleotides, display at least 65% inhibition: 159-179, 282-305, 290-327, 375-394, 381-465, 538-676, 1139-1287, or 1555-2203.

In certain embodiments, the following nucleotide regions of SEQ ID NO: 1, when targeted by antisense compounds or oligonucleotides, display at least 70% inhibition: 159-179, 284-305, 292-321, 308-327, 446-465, 538-640, 625-676, 1139-1287, or 1891-2192.

In certain embodiments, the following nucleotide regions of SEQ ID NO: 1, when targeted by antisense compounds or oligonucleotides, display at least 75% inhibition: 159-179, 292-311, 298-319, 558-640, 627-676, 1139-1207, 1891-1998, or 2111-2176.

In certain embodiments, the following nucleotide regions of SEQ ID NO: 1, when targeted by antisense compounds or oligonucleotides, display at least 80% inhibition: 159-178, 292-311, 298-317, 621-640, 629-668, 655-674, 1139-1158, 1143-1162, 1149-1170, 1891-1998, or 2111-2176.

In certain embodiments, the following nucleotide regions of SEQ ID NO: 1, when targeted by antisense compounds or oligonucleotides, display at least 85% inhibition: 159-178, 292-311, 298-317, 629-652, 637-664, 2111-2136, or 2157-2176.

In certain embodiments, the following nucleotide regions of SEQ ID NO: 1, when targeted by antisense compounds or oligonucleotides, display at least 90% inhibition: 631-650, 643-662, or 2157-2176.

In certain embodiments, an antisense compound or oligonucleotide targeted to a TGF-beta1 nucleic acid target the following nucleotide regions of SEQ ID NO 2: 3058-3286, 3891-3910, 4228-4725, 4302-4555, 4744-5053, 5615-5680, 5996-6933, 6423-6528, 6452-6471, 6676-6933, 6747-6837, 7661-8374, 9216-9893, 10754-12857, 10754-10927, 11275-11936, 12119-12842, 14052-14119, 14083-14119, 14879-15112, 14879-14978, 15020-15112, 15205-15253, 15636-15907, 15717-15907, 18043-18203, 18114-18203, 18953-19168, 18953-18975, 19046-19065, 19149-19168, 19512-19531, 20285-23427, 20285-21133, 20285-20902, 21934-22892, or 23222-23367.

In certain embodiments, antisense compounds or oligonucleotides target a range of a TGF-beta1 nucleic acid. In certain embodiments, such compounds or oligonucleotides targeted to a range of a TGF-beta1 nucleic acid have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobase portion complementary to an equal length portion of a region recited herein. In certain embodiments, such compounds or oligonucleotides, which are targeted to a region of a TGF-beta1 nucleic acid and have a portion that is complementary to an equal length portion of the region, target the following nucleotide regions of SEQ ID NO: 2: 3058-3286, 3891-3910, 4228-4725, 4302-4555, 4744-5053, 5615-5680, 5996-6933, 6423-6528, 6452-6471, 6676-6933, 6747-6837, 7661-8374, 9216-9893, 10754-12857, 10754-10927, 11275-11936, 12119-12842, 14052-14119, 14083-14119, 14879-15112, 14879-14978, 15020-15112, 15205-15253, 15636-15907, 15717-15907, 18043-18203, 18114-18203, 18953-19168, 18953-18975, 19046-19065, 19149-19168, 19512-19531, 20285-23427, 20285-21133, 20285-20902, 21934-22892, or 23222-23367.

In certain embodiments, the following nucleotide regions of SEQ ID NO: 2, when targeted by antisense compounds or oligonucleotides, display at least 60% inhibition: 3058-3077, 3267-3286, 3891-3910, 4302-4321, 4536-4555, 6452-6471, 6509-6528, 6676-6695, 6747-6766, 6818-6837, 6914-6933, 7661-7680, 8355-8374, 9362-9381, 10908-10927, 11275-11294, 11917-11936, 12119-12138, 14083-14102, 14100-14119, 14893-14912, 14959-14978, 15020-15039, 15093-15112, 15205-15224, 15234-15253, 15636-15655, 15717-15736, 15819-15838, 15888-15907, 18114-18133, 18184-18203, 18956-18975, 19046-19065, 19149-19168, 19512-19531, 20285-20304, 20883-20902, 21934-21953, 22018-22037, 22873-22892, or 23348-23367.

In certain embodiments, the following nucleotide regions of SEQ ID NO: 2, when targeted by antisense compounds or oligonucleotides, display at least 65% inhibition: 3058-3077, 3267-3286, 3891-3910, 4536-4555, 6452-6471, 6509-6528, 6676-6695, 6747-6766, 6818-6837, 7661-7680, 8355-8374, 10908-10927, 11275-11294, 11917-11936, 14083-14102, 14100-14119, 14893-14912, 14959-14978, 15020-15039, 15205-15224, 15234-15253, 15636-15655, 15717-15736, 15819-15838, 15888-15907, 18114-18133, 18184-18203, 19046-19065, 19512-19531, 20285-20304, 20883-20902, 21934-21953, 22018-22037, or 22873-22892.

In certain embodiments, the following nucleotide regions of SEQ ID NO: 2, when targeted by antisense compounds or oligonucleotides, display at least 70% inhibition: 3058-3077, 3267-3286, 4536-4555, 6452-6471, 6747-6766, 6818-6837, 7661-7680, 8355-8374, 11275-11294, 11917-11936, 14083-14102, 14893-14912, 15020-15039, 15205-15224, 15717-15736, 15819-15838, 15888-15907, 18114-18133, 18184-18203, 19046-19065, 19512-19531, 20285-20304, 20883-20902, 21934-21953, 22018-22037, or 22873-22892.

In certain embodiments, the following nucleotide regions of SEQ ID NO: 2, when targeted by antisense compounds or oligonucleotides, display at least 75% inhibition: 3267-3286, 4536-4555, 6452-6471, 6818-6837, 7661-7680, 11275-11294, 14083-14102, 14893-14912, 15020-15039, 15205-15224, 18184-18203, 19512-19531, 20285-20304, 20883-20902, 21934-21953, or 22018-22037.

In certain embodiments, the following nucleotide regions of SEQ ID NO: 2, when targeted by antisense compounds or oligonucleotides, display at least 80% inhibition: 3267-3286, 4536-4555, 6452-6471, 6818-6837, 7661-7680, 15020-15039, 15205-15224, 18184-18203, 19512-19531, 20285-20304, 21934-21953, or 22018-22037.

In certain embodiments, the following nucleotide regions of SEQ ID NO: 2, when targeted by antisense compounds or oligonucleotides, display at least 85% inhibition: 15205-15224 or 18184-18203.

In certain embodiments, the following antisense compounds or oligonucleotides target a region of a TGF-beta1 nucleic acid and effect at least a 60% inhibition of a TGF-beta1 mRNA: Oligo IDs 413967, 413970, 413971, 413972, 413974, 413975, 413976, 413978, 413979, 413980, 413981, 413982, 413983, 413984, 413985, 413986, 413987, 413988, 413991, 413992, 413994, 413995, 413999, 414000, 414001, 414002, 414003, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414030, 414031, 414032, 414033, 414034, 414035, 414036, 414037, 414038, 414039, 414040, 414041, 414042, 414043, 414045, 414046, 414048, 414050, 414058, 414059, 414061, 414062, 14063, 414064, 414066, 414067, 414069, 414073, 414075, 414077, 414079, 414084, 414085, 414087, 414088, 414090, 414091, 414092, 414093, 414094, 414096, 414097, 414098, 414101, 414102, 414104, 414106, 414108, 414109, 414111, 414113, 414116, 414117, 414118, and 414121.

In certain embodiments, the following antisense compounds or oligonucleotides target a region of a TGF-beta1 nucleic acid and effect at least a 65% inhibition of a TGF-beta1 mRNA: Oligo IDs 413970, 413971, 413974, 413975, 413976, 413978, 413979, 413980, 413981, 413982, 413983, 413984, 413985, 413986, 413987, 413991, 413994, 413995, 413999, 414000, 414001, 414002, 414003, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414031, 414032, 414033, 414034, 414035, 414036, 414037, 414038, 414039, 414040, 414041, 414042, 414045, 414046, 414050, 414058, 414059, 414061, 414062, 414063, 414066, 414067, 414073, 414075, 414077, 414084, 414085, 414087, 414088, 414090, 414092, 414093, 414094, 414096, 414097, 414098, 414101, 414102, 414106, 414109, 414111, 414113, 414116, 414117, and 414118.

In certain embodiments, the following antisense compounds or oligonucleotides target a region of a TGF-beta1 nucleic acid and effect at least 70% inhibition of a TGF-beta1 mRNA: Oligo IDs 413970, 413971, 413975, 413976, 413979, 413980, 413981, 413982, 413983, 413984, 413987, 413995, 413999, 414000, 414001, 414002, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414032, 414033, 414035, 414036, 414037, 414038, 414039, 414040, 414041, 414045, 414050, 414058, 414062, 414063, 414066, 414067, 414075, 414077, 414084, 414087, 414090, 414092, 414096, 414097, 414098, 414101, 414102, 414106, 414109, 414111, 414113, 414116, 414117, and 414118.

In certain embodiments, the following antisense compounds or oligonucleotides target a region of a TGF-beta1 nucleic acid and effect at least 75% inhibition of a TGF-beta1 mRNA: Oligo IDs 413970, 413971, 413979, 413982, 413983, 414000, 414001, 414002, 414005, 414006, 414007, 414008, 414009, 414010, 414011, 414012, 414013, 414014, 414015, 414016, 414017, 414018, 414019, 414022, 414023, 414024, 414025, 414026, 414027, 414028, 414032, 414033, 414035, 414036, 414037, 414038, 414039, 414040, 414045, 414050, 414058, 414063, 414066, 414075, 414084, 414087, 414090, 414092, 414102, 414109, 414111, 414113, 414116, and 414117.

In certain embodiments, the following antisense compounds or oligonucleotides target a region of a TGF-beta1 nucleic acid and effect at least 80% inhibition of a TGF-beta1 mRNA: Oligo IDs 413970, 413979, 413982, 414002, 414006, 414007, 414008, 414009, 414010, 414011, 414012, 414013, 414014, 414015, 414018, 414022, 414024, 414026, 414027, 414032, 414033, 414035, 414036, 414037, 414038, 414039, 414040, 414045, 414050, 414058, 414063, 414066, 414090, 414092, 414102, 414109, 414111, 414116, and 414117.

In certain embodiments, the following antisense compounds or oligonucleotides target a region of a TGF-beta1 nucleic acid and effect at least 85% inhibition of a TGF-beta1 mRNA: Oligo IDs 413970, 413979, 413982, 414006, 414007, 414008, 414010, 414011, 414012, 414013, 414014, 414035, 414036, 414037, 414038, 414040, 414092, and 414102.

In certain embodiments, the following antisense compounds or oligonucleotides target a region of a TGF-beta1 nucleic acid and effect at least 90% inhibition of a TGF-beta1 mRNA: Oligo IDs 414007, 414013, and 414040.

In certain embodiments, a target region is nucleotides 1-20 of SEQ ID NO: 1. In certain embodiments, an antisense compound is targeted to nucleotides 1-20 of SEQ ID NO: 1. In certain embodiments, an antisense compound targeted to a TGF-beta1 nucleic acid comprises a nucleobase sequence of SEQ ID NO: 4. In certain such embodiments, an antisense compound targeted to nucleotides 1-20 of SEQ ID NO: 1 is Oligo ID: 413967.

In certain embodiments, a target region is nucleotides 159-255 of SEQ ID NO: 1. In certain embodiments, an antisense compound is targeted to nucleotides 159-255 of SEQ ID NO: 1. In certain embodiments, an antisense compound targeted to a TGF-beta1 nucleic acid comprises a nucleobase sequence selected from SEQ ID NOs: 7, 8, or 9. In certain such embodiments, an antisense compound targeted to nucleotides 159-255 of SEQ ID NO: 1 is selected from Oligo IDs: 413970, 413971 or 413972.

In certain embodiments, a target region is nucleotides 282-305 of SEQ ID NO: 1. In certain embodiments, an antisense compound is targeted to nucleotides 282-305 of SEQ ID NO: 1. In certain embodiments, an antisense compound targeted to a TGF-beta1 nucleic acid comprises a nucleobase sequence selected from SEQ ID NOs: 11, 12, or 13. In certain such embodiments, an antisense compound targeted to nucleotides 282-305 of SEQ ID NO: 1 is selected from Oligo IDs: 413974, 413975, or 413976.

In certain embodiments, a target region is nucleotides 290-363 of SEQ ID NO: 1. In certain embodiments, an antisense compound is targeted to nucleotides 290-363 of SEQ ID NO: 1. In certain embodiments, an antisense compound targeted to a TGF-beta1 nucleic acid comprises a nucleobase sequence selected from SEQ ID NOs: 15-25. In certain such embodiments, an antisense compound targeted to nucleotides 290-363 of SEQ ID NO: 1 is selected from Oligo IDs: 413978, 413979, 413980, 413981, 413982, 413983, 413984, 413985, 413986, 413987 or 413988.

In certain embodiments, a target region is nucleotides 292-321 of SEQ ID NO: 1. In certain embodiments, an antisense compound is targeted to nucleotides 292-321 of SEQ ID NO: 1. In certain embodiments, an antisense compound targeted to a TGF-beta1 nucleic acid comprises a nucleobase sequence selected from SEQ ID NOs: 16-21. In certain such embodiments, an antisense compound targeted to nucleotides 292-321 of SEQ ID NO: 1 is selected from Oligo IDs: 413979, 413980, 413981, 413982, 413983, or 413984.

In certain embodiments, a target region is nucleotides 375-396 of SEQ ID NO: 1. In certain embodiments, an antisense compound is targeted to nucleotides 375-396 of SEQ ID NO: 1. In certain embodiments, an antisense compound targeted to a TGF-beta1 nucleic acid comprises a nucleobase sequence selected from SEQ ID NOs: 28 or 29. In certain such embodiments, an antisense compound targeted to nucleotides 375-396 of SEQ ID NO: 1 is selected from Oligo IDs: 413991 or 413992.

In certain embodiments, a target region is nucleotides 381-465 of SEQ ID NO: 1. In certain embodiments, an antisense compound is targeted to nucleotides 381-465 of SEQ ID NO: 1. In certain embodiments, an antisense compound targeted to a TGF-beta1 nucleic acid comprises a nucleobase sequence selected from SEQ ID NOs: 31 or 32. In certain such embodiments, an antisense compound targeted to nucleotides 381-465 of SEQ ID NO: 1 is selected from Oligo IDs: 413994 or 413995.

In certain embodiments, a target region is nucleotides 538-676 of SEQ ID NO: 1. In certain embodiments, an antisense compound is targeted to nucleotides 538-676 of SEQ ID NO: 1. In certain embodiments, an antisense compound targeted to a TGF-beta1 nucleic acid comprises a nucleobase sequence selected from SEQ ID NOs: 36-56. In certain such embodiments, an antisense compound targeted to nucleotides 538-676 of SEQ ID NO: 1 is selected from Oligo IDs: 413999, 414000, 414001, 414002, 414003, 414004, 414005, 414006, 414007, 414008, 414009, 414010, 414011, 414012, 414013, 414014, 414015, 414016, 414017, 414018, or 414019.

In certain embodiments, a target region is nucleotides 538-640 of SEQ ID NO: 1. In certain embodiments, an antisense compound is targeted to nucleotides 538-640 of SEQ ID NO: 1. In certain embodiments, an antisense compound targeted to a TGF-beta1 nucleic acid comprises a nucleobase sequence selected from SEQ ID NOs: 36-39. In certain such embodiments, an antisense compound targeted to nucleotides 538-640 of SEQ ID NO: 1 is selected from Oligo IDs: 413999, 414000, 414001, or 414002.

In certain embodiments, a target region is nucleotides 558-640 of SEQ ID NO: 1. In certain embodiments, an antisense compound is targeted to nucleotides 558-640 of SEQ ID NO: 1. In certain embodiments, an antisense compound targeted to a TGF-beta1 nucleic acid comprises a nucleobase sequence selected from SEQ ID NOs: 37-39. In certain such embodiments, an antisense compound targeted to nucleotides 558-640 of SEQ ID NO: 1 is selected from Oligo IDs: 414000, 414001, or 414002.

In certain embodiments, a target region is nucleotides 625-676 of SEQ ID NO: 1. In certain embodiments, an antisense compound is targeted to nucleotides 625-676 of SEQ ID NO: 1. In certain embodiments, an antisense compound targeted to a TGF-beta1 nucleic acid comprises a nucleobase sequence selected from SEQ ID NOs: 41-56. In certain such embodiments, an antisense compound targeted to nucleotides 625-676 of SEQ ID NO: 1 is selected from Oligo IDs: 414004, 414005, 414006, 414007, 414008, 414009, 414010, 414011, 414012, 414013, 414014, 414015, 414016, 414017, 414018, or 414019.

In certain embodiments, a target region is nucleotides 627-676 of SEQ ID NO: 1. In certain embodiments, an antisense compound is targeted to nucleotides 627-676 of SEQ ID NO: 1. In certain embodiments, an antisense compound targeted to a TGF-beta1 nucleic acid comprises a nucleobase sequence selected from SEQ ID NOs: 42-56. In certain such embodiments, an antisense compound targeted to nucleotides 627-676 of SEQ ID NO: 1 is selected from Oligo IDs: 414005, 414006, 414007, 414008, 414009, 414010, 414011, 414012, 414013, 414014, 414015, 414016, 414017, 414018, or 414019.

In certain embodiments, a target region is nucleotides 629-668 of SEQ ID NO: 1. In certain embodiments, an antisense compound is targeted to nucleotides 629-668 of SEQ ID NO: 1. In certain embodiments, an antisense compound targeted to a TGF-beta1 nucleic acid comprises a nucleobase sequence selected from SEQ ID NOs: 43-52. In certain such embodiments, an antisense compound targeted to nucleotides 629-668 of SEQ ID NO: 1 is selected from Oligo IDs: 414006, 414007, 414008, 414009, 414010, 414011, 414012, 414013, 414014, or 414015.

In certain embodiments, a target region is nucleotides 631-652 of SEQ ID NO: 1. In certain embodiments, an antisense compound is targeted to nucleotides 631-652 of SEQ ID NO: 1. In certain embodiments, an antisense compound targeted to a TGF-beta1 nucleic acid comprises a nucleobase sequence selected from SEQ ID NOs: 44 or 45. In certain such embodiments, an antisense compound targeted to nucleotides 631-652 of SEQ ID NO: 1 is selected from Oligo IDs: 414007 or 414008.

In certain embodiments, a target region is nucleotides 637-664 of SEQ ID NO: 1. In certain embodiments, an antisense compound is targeted to nucleotides 637-664 of SEQ ID NO: 1. In certain embodiments, an antisense compound targeted to a TGF-beta1 nucleic acid comprises a nucleobase sequence selected from SEQ ID NOs: 47-51. In certain such embodiments, an antisense compound targeted to nucleotides 637-664 of SEQ ID NO: 1 is selected from Oligo IDs: 414010, 414011, 414012, 414013, or 414014.

In certain embodiments, a target region is nucleotides 1139-2308 of SEQ ID NO: 1. In certain embodiments, an antisense compound is targeted to nucleotides 1139-2308 of SEQ ID NO: 1. In certain embodiments, an antisense compound targeted to a TGF-beta1 nucleic acid comprises a nucleobase sequence selected from SEQ ID NOs: 58-79. In certain such embodiments, an antisense compound targeted to nucleotides 1139-2308 of SEQ ID NO: 1 is selected from Oligo IDs: 414022, 414023, 414024, 414025, 414026, 414027, 414028, 414029, 414030, 414031, 414032, 414033, 414034, 414035, 414036, 414037, 414038, 414039, 414040, 414041, 414042, or 414043.

In certain embodiments, a target region is nucleotides 1139-1287 of SEQ ID NO: 1. In certain embodiments, an antisense compound is targeted to nucleotides 1139-1287 of SEQ ID NO: 1. In certain embodiments, an antisense compound targeted to a TGF-beta1 nucleic acid comprises a nucleobase sequence selected from SEQ ID NOs: 58-73. In certain such embodiments, an antisense compound targeted to nucleotides 1139-1287 of SEQ ID NO: 1 is selected from Oligo IDs: 414022, 414023, 414024, 414025, 414026, 414027, 414028, or 414029.

In certain embodiments, a target region is nucleotides 2111-2176 of SEQ ID NO: 1. In certain embodiments, an antisense compound is targeted to nucleotides 2111-2176 of SEQ ID NO: 1. In certain embodiments, an antisense compound targeted to a TGF-beta1 nucleic acid comprises a nucleobase sequence selected from SEQ ID NOs: 71-76. In certain such embodiments, an antisense compound targeted to nucleotides 2111-2176 of SEQ ID NO: 1 is selected from Oligo IDs: 414035, 414036, 414037, 414038, 414039, or 414040.

In certain embodiments, a target region is nucleotides 3058-3077 of SEQ ID NO: 2. In certain embodiments, an antisense compound is targeted to nucleotides 3058-3077 of SEQ ID NO: 2. In certain embodiments, an antisense compound targeted to a TGF-beta1 nucleic acid comprises a nucleotide sequence of SEQ ID NO: 81. In certain such embodiments, an antisense compound targeted to nucleotides 3058-3077 of SEQ ID NO: 2 is Oligo ID: 414021.

In certain embodiments, a target region is nucleotides 3267-3286 of SEQ ID NO: 2. In certain embodiments, an antisense compound is targeted to nucleotides 3267-3286 of SEQ ID NO: 2. In certain embodiments, an antisense compound targeted to a TGF-beta1 nucleic acid comprises a nucleotide sequence of SEQ ID NO: 82. In certain such embodiments, an antisense compound targeted to nucleotides 3267-3286 of SEQ ID NO: 2 is Oligo ID: 414045.

In certain embodiments, a target region is nucleotides 3891-3910 of SEQ ID NO: 2. In certain embodiments, an antisense compound is targeted to nucleotides 3891-3910 of SEQ ID NO: 2. In certain embodiments, an antisense compound targeted to a TGF-beta1 nucleic acid comprises a nucleotide sequence of SEQ ID NO: 83. In certain such embodiments, an antisense compound targeted to nucleotides 3891-3910 of SEQ ID NO: 2 is Oligo ID: 414046.

In certain embodiments, a target region is nucleotides 4302-4321 of SEQ ID NO: 2. In certain embodiments, an antisense compound is targeted to nucleotides 4302-4321 of SEQ ID NO: 2. In certain embodiments, an antisense compound targeted to a TGF-beta1 nucleic acid comprises a nucleotide sequence of SEQ ID NO: 85. In certain such embodiments, an antisense compound targeted to nucleotides 4302-4321 of SEQ ID NO: 2 is Oligo ID: 414048.

In certain embodiments, a target region is nucleotides 4536-4555 of SEQ ID NO: 2. In certain embodiments, an antisense compound is targeted to nucleotides 4536-4555 of SEQ ID NO: 2. In certain embodiments, an antisense compound targeted to a TGF-beta1 nucleic acid comprises a nucleotide sequence of SEQ ID NO: 87. In certain such embodiments, an antisense compound targeted to nucleotides 4536-4555 of SEQ ID NO: 2 is Oligo ID: 414050.

In certain embodiments, a target region is nucleotides 6452-6471 of SEQ ID NO: 2. In certain embodiments, an antisense compound is targeted to nucleotides 6452-6471 of SEQ ID NO: 2. In certain embodiments, an antisense compound targeted to a TGF-beta1 nucleic acid comprises a nucleotide sequence of SEQ ID NO: 95. In certain such embodiments, an antisense compound targeted to nucleotides 6452-6471 of SEQ ID NO: 2 is Oligo ID: 414058.

In certain embodiments, a target region is nucleotides 6509-6528 of SEQ ID NO: 2. In certain embodiments, an antisense compound is targeted to nucleotides 6509-6528 of SEQ ID NO: 2. In certain embodiments, an antisense compound targeted to a TGF-beta1 nucleic acid comprises a nucleotide sequence of SEQ ID NO: 96. In certain such embodiments, an antisense compound targeted to nucleotides 6509-6528 of SEQ ID NO: 2 is Oligo ID: 414059.

In certain embodiments, a target region is nucleotides 6676-6695 of SEQ ID NO: 2. In certain embodiments, an antisense compound is targeted to nucleotides 6676-6695 of SEQ ID NO: 2. In certain embodiments, an antisense compound targeted to a TGF-beta1 nucleic acid comprises a nucleotide sequence of SEQ ID NO: 98. In certain such embodiments, an antisense compound targeted to nucleotides 6676-6695 of SEQ ID NO: 2 is Oligo ID: 414061.

In certain embodiments, a target region is nucleotides 6747-6766 of SEQ ID NO: 2. In certain embodiments, an antisense compound is targeted to nucleotides 6747-6766 of SEQ ID NO: 2. In certain embodiments, an antisense compound targeted to a TGF-beta1 nucleic acid comprises a nucleotide sequence of SEQ ID NO: 99. In certain such embodiments, an antisense compound targeted to nucleotides 6747-6766 of SEQ ID NO: 2 is Oligo ID: 414062.

In certain embodiments, a target region is nucleotides 6818-6837 of SEQ ID NO: 2. In certain embodiments, an antisense compound is targeted to nucleotides 6818-6837 of SEQ ID NO: 2. In certain embodiments, an antisense compound targeted to a TGF-beta1 nucleic acid comprises a nucleotide sequence of SEQ ID NO: 100. In certain such embodiments, an antisense compound targeted to nucleotides 6818-6837 of SEQ ID NO: 2 is Oligo ID: 414063.

In certain embodiments, a target region is nucleotides 6914-6933 of SEQ ID NO: 2. In certain embodiments, an antisense compound is targeted to nucleotides 6914-6933 of SEQ ID NO: 2. In certain embodiments, an antisense compound targeted to a TGF-beta1 nucleic acid comprises a nucleotide sequence of SEQ ID NO: 101. In certain such embodiments, an antisense compound targeted to nucleotides 6914-6933 of SEQ ID NO: 2 is Oligo ID: 414064.

In certain embodiments, a target region is nucleotides 7661-7680 of SEQ ID NO: 2. In certain embodiments, an antisense compound is targeted to nucleotides 7661-7680 of SEQ ID NO: 2. In certain embodiments, an antisense compound targeted to a TGF-beta1 nucleic acid comprises a nucleotide sequence of SEQ ID NO: 103. In certain such embodiments, an antisense compound targeted to nucleotides 7661-7680 of SEQ ID NO: 2 is Oligo ID: 414066.

In certain embodiments, a target region is nucleotides 8355-8374 of SEQ ID NO: 2. In certain embodiments, an antisense compound is targeted to nucleotides 8355-8374 of SEQ ID NO: 2. In certain embodiments, an antisense compound targeted to a TGF-beta1 nucleic acid comprises a nucleotide sequence of SEQ ID NO: 104. In certain such embodiments, an antisense compound targeted to nucleotides 8355-8374 of SEQ ID NO: 2 is Oligo ID: 414067.

In certain embodiments, a target region is nucleotides 9362-9381 of SEQ ID NO: 2. In certain embodiments, an antisense compound is targeted to nucleotides 9362-9381 of SEQ ID NO: 2. In certain embodiments, an antisense compound targeted to a TGF-beta1 nucleic acid comprises a nucleotide sequence of SEQ ID NO: 106. In certain such embodiments, an antisense compound targeted to nucleotides 9362-9381 of SEQ ID NO: 2 is Oligo ID: 414069.

In certain embodiments, a target region is nucleotides 10908-10927 of SEQ ID NO: 2. In certain embodiments, an antisense compound is targeted to nucleotides 10908-10927 of SEQ ID NO: 2. In certain embodiments, an antisense compound targeted to a TGF-beta1 nucleic acid comprises a nucleotide sequence of SEQ ID NO: 110. In certain such embodiments, an antisense compound targeted to nucleotides 10908-10927 of SEQ ID NO: 2 is Oligo ID: 414073.

In certain embodiments, a target region is nucleotides 11275-11294 of SEQ ID NO: 2. In certain embodiments, an antisense compound is targeted to nucleotides 11275-11294 of SEQ ID NO: 2. In certain embodiments, an antisense compound targeted to a TGF-beta1 nucleic acid comprises a nucleotide sequence of SEQ ID NO: 112. In certain such embodiments, an antisense compound targeted to nucleotides 11275-11294 of SEQ ID NO: 2 is Oligo ID: 414075.

In certain embodiments, a target region is nucleotides 11917-11936 of SEQ ID NO: 2. In certain embodiments, an antisense compound is targeted to nucleotides 11917-11936 of SEQ ID NO: 2. In certain embodiments, an antisense compound targeted to a TGF-beta1 nucleic acid comprises a nucleotide sequence of SEQ ID NO: 114. In certain such embodiments, an antisense compound targeted to nucleotides 11917-11936 of SEQ ID NO: 2 is Oligo ID: 414077.

In certain embodiments, a target region is nucleotides 12119-12138 of SEQ ID NO: 2. In certain embodiments, an antisense compound is targeted to nucleotides 12119-12138 of SEQ ID NO: 2. In certain embodiments, an antisense compound targeted to a TGF-beta1 nucleic acid comprises a nucleotide sequence of SEQ ID NO: 116. In certain such embodiments, an antisense compound targeted to nucleotides 12119-12138 of SEQ ID NO: 2 is Oligo ID: 414079.

In certain embodiments, a target region is nucleotides 14083-14102 of SEQ ID NO: 2. In certain embodiments, an antisense compound is targeted to nucleotides 14083-14102 of SEQ ID NO: 2. In certain embodiments, an antisense compound targeted to a TGF-beta1 nucleic acid comprises a nucleotide sequence of SEQ ID NO: 121. In certain such embodiments, an antisense compound targeted to nucleotides 14083-14102 of SEQ ID NO: 2 is Oligo ID: 414084.

In certain embodiments, a target region is nucleotides 14100-14119 of SEQ ID NO: 2. In certain embodiments, an antisense compound is targeted to nucleotides 14100-14119 of SEQ ID NO: 2. In certain embodiments, an antisense compound targeted to a TGF-beta1 nucleic acid comprises a nucleotide sequence of SEQ ID NO: 122. In certain such embodiments, an antisense compound targeted to nucleotides 14100-14119 of SEQ ID NO: 2 is Oligo ID: 414085.

In certain embodiments, a target region is nucleotides 14893-14912 of SEQ ID NO: 2. In certain embodiments, an antisense compound is targeted to nucleotides 14893-14912 of SEQ ID NO: 2. In certain embodiments, an antisense compound targeted to a TGF-beta1 nucleic acid comprises a nucleotide sequence of SEQ ID NO: 124. In certain such embodiments, an antisense compound targeted to nucleotides 14893-14912 of SEQ ID NO: 2 is Oligo ID: 414087.

In certain embodiments, a target region is nucleotides 14959-14978 of SEQ ID NO: 2. In certain embodiments, an antisense compound is targeted to nucleotides 14959-14978 of SEQ ID NO: 2. In certain embodiments, an antisense compound targeted to a TGF-beta1 nucleic acid comprises a nucleotide sequence of SEQ ID NO: 125. In certain such embodiments, an antisense compound targeted to nucleotides 14959-14978 of SEQ ID NO: 2 is Oligo ID: 414088.

In certain embodiments, a target region is nucleotides 15020-15039 of SEQ ID NO: 2. In certain embodiments, an antisense compound is targeted to nucleotides 15020-15039 of SEQ ID NO: 2. In certain embodiments, an antisense compound targeted to a TGF-beta1 nucleic acid comprises a nucleotide sequence of SEQ ID NO: 127. In certain such embodiments, an antisense compound targeted to nucleotides 15020-15039 of SEQ ID NO: 2 is Oligo ID: 414090.

In certain embodiments, a target region is nucleotides 15093-15112 of SEQ ID NO: 2. In certain embodiments, an antisense compound is targeted to nucleotides 15093-15112 of SEQ ID NO: 2. In certain embodiments, an antisense compound targeted to a TGF-beta1 nucleic acid comprises a nucleotide sequence of SEQ ID NO: 128. In certain such embodiments, an antisense compound targeted to nucleotides 15093-15112 of SEQ ID NO: 2 is Oligo ID: 414091.

In certain embodiments, a target region is nucleotides 15205-15224 of SEQ ID NO: 2. In certain embodiments, an antisense compound is targeted to nucleotides 15205-15224 of SEQ ID NO: 2. In certain embodiments, an antisense compound targeted to a TGF-beta1 nucleic acid comprises a nucleotide sequence of SEQ ID NO: 129. In certain such embodiments, an antisense compound targeted to nucleotides 15205-15224 of SEQ ID NO: 2 is Oligo ID: 414092.

In certain embodiments, a target region is nucleotides 15234-15253 of SEQ ID NO: 2. In certain embodiments, an antisense compound is targeted to nucleotides 15234-15253 of SEQ ID NO: 2. In certain embodiments, an antisense compound targeted to a TGF-beta1 nucleic acid comprises a nucleotide sequence of SEQ ID NO: 130. In certain such embodiments, an antisense compound targeted to nucleotides 15234-15253 of SEQ ID NO: 2 is Oligo ID: 414093.

In certain embodiments, a target region is nucleotides 15636-15655 of SEQ ID NO: 2. In certain embodiments, an antisense compound is targeted to nucleotides 15636-15655 of SEQ ID NO: 2. In certain embodiments, an antisense compound targeted to a TGF-beta1 nucleic acid comprises a nucleotide sequence of SEQ ID NO: 131. In certain such embodiments, an antisense compound targeted to nucleotides 15636-15655 of SEQ ID NO: 2 is Oligo ID: 414094.

In certain embodiments, a target region is nucleotides 15717-15736 of SEQ ID NO: 2. In certain embodiments, an antisense compound is targeted to nucleotides 15717-15736 of SEQ ID NO: 2. In certain embodiments, an antisense compound targeted to a TGF-beta1 nucleic acid comprises a nucleotide sequence of SEQ ID NO: 133. In certain such embodiments, an antisense compound targeted to nucleotides 15717-15736 of SEQ ID NO: 2 is Oligo ID: 414096.

In certain embodiments, a target region is nucleotides 15819-15838 of SEQ ID NO: 2. In certain embodiments, an antisense compound is targeted to nucleotides 15819-15838 of SEQ ID NO: 2. In certain embodiments, an antisense compound targeted to a TGF-beta1 nucleic acid comprises a nucleotide sequence of SEQ ID NO: 134. In certain such embodiments, an antisense compound targeted to nucleotides 15819-15838 of SEQ ID NO: 2 is Oligo ID: 414097.

In certain embodiments, a target region is nucleotides 15888-15907 of SEQ ID NO: 2. In certain embodiments, an antisense compound is targeted to nucleotides 15888-15907 of SEQ ID NO: 2. In certain embodiments, an antisense compound targeted to a TGF-beta1 nucleic acid comprises a nucleotide sequence of SEQ ID NO: 135. In certain such embodiments, an antisense compound targeted to nucleotides 15888-15907 of SEQ ID NO: 2 is Oligo ID: 414098.

In certain embodiments, a target region is nucleotides 18114-18133 of SEQ ID NO: 2. In certain embodiments, an antisense compound is targeted to nucleotides 18114-18133 of SEQ ID NO: 2. In certain embodiments, an antisense compound targeted to a TGF-beta1 nucleic acid comprises a nucleotide sequence of SEQ ID NO: 138. In certain such embodiments, an antisense compound targeted to nucleotides 18114-18133 of SEQ ID NO: 2 is Oligo ID: 414101.

In certain embodiments, a target region is nucleotides 18184-18203 of SEQ ID NO: 2. In certain embodiments, an antisense compound is targeted to nucleotides 8184-18203 of SEQ ID NO: 2. In certain embodiments, an antisense compound targeted to a TGF-beta1 nucleic acid comprises a nucleotide sequence of SEQ ID NO: 139. In certain such embodiments, an antisense compound targeted to nucleotides 18184-18203 of SEQ ID NO: 2 is Oligo ID: 414102.

In certain embodiments, a target region is nucleotides 18956-18975 of SEQ ID NO: 2. In certain embodiments, an antisense compound is targeted to nucleotides 18956-18975 of SEQ ID NO: 2. In certain embodiments, an antisense compound targeted to a TGF-beta1 nucleic acid comprises a nucleotide sequence of SEQ ID NO: 141. In certain such embodiments, an antisense compound targeted to nucleotides 18956-18975 of SEQ ID NO: 2 is Oligo ID: 414104.

In certain embodiments, a target region is nucleotides 19046-19065 of SEQ ID NO: 2. In certain embodiments, an antisense compound is targeted to nucleotides 19046-19065 of SEQ ID NO: 2. In certain embodiments, an antisense compound targeted to a TGF-beta1 nucleic acid comprises a nucleotide sequence of SEQ ID NO: 143. In certain such embodiments, an antisense compound targeted to nucleotides 19046-19065 of SEQ ID NO: 2 is Oligo ID: 414106.

In certain embodiments, a target region is nucleotides 19149-19168 of SEQ ID NO: 2. In certain embodiments, an antisense compound is targeted to nucleotides 19149-19168 of SEQ ID NO: 2. In certain embodiments, an antisense compound targeted to a TGF-beta1 nucleic acid comprises a nucleotide sequence of SEQ ID NO: 145. In certain such embodiments, an antisense compound targeted to nucleotides 19149-19168 of SEQ ID NO: 2 is Oligo ID: 414108.

In certain embodiments, a target region is nucleotides 19512-19531 of SEQ ID NO: 2. In certain embodiments, an antisense compound is targeted to nucleotides 19512-19531 of SEQ ID NO: 2. In certain embodiments, an antisense compound targeted to a TGF-beta1 nucleic acid comprises a nucleotide sequence of SEQ ID NO: 146. In certain such embodiments, an antisense compound targeted to nucleotides 19512-19531 of SEQ ID NO: 2 is Oligo ID: 414109.

In certain embodiments, a target region is nucleotides 20285-20304 of SEQ ID NO: 2. In certain embodiments, an antisense compound is targeted to nucleotides 20285-20304 of SEQ ID NO: 2. In certain embodiments, an antisense compound targeted to a TGF-beta1 nucleic acid comprises a nucleotide sequence of SEQ ID NO: 148. In certain such embodiments, an antisense compound targeted to nucleotides 20285-20304 of SEQ ID NO: 2 is Oligo ID: 414111.

In certain embodiments, a target region is nucleotides 20883-20902 of SEQ ID NO: 2. In certain embodiments, an antisense compound is targeted to nucleotides 20883-20902 of SEQ ID NO: 2. In certain embodiments, an antisense compound targeted to a TGF-beta1 nucleic acid comprises a nucleotide sequence of SEQ ID NO: 150. In certain such embodiments, an antisense compound targeted to nucleotides 20883-20902 of SEQ ID NO: 2 is Oligo ID: 414113.

In certain embodiments, a target region is nucleotides 21934-21953 of SEQ ID NO: 2. In certain embodiments, an antisense compound is targeted to nucleotides 21934-21953 of SEQ ID NO: 2. In certain embodiments, an antisense compound targeted to a TGF-beta1 nucleic acid comprises a nucleotide sequence of SEQ ID NO: 153. In certain such embodiments, an antisense compound targeted to nucleotides 21934-21953 of SEQ ID NO: 2 is Oligo ID: 414116.

In certain embodiments, a target region is nucleotides 22018-22037 of SEQ ID NO: 2. In certain embodiments, an antisense compound is targeted to nucleotides 22018-22037 of SEQ ID NO: 2. In certain embodiments, an antisense compound targeted to a TGF-beta1 nucleic acid comprises a nucleotide sequence of SEQ ID NO: 154. In certain such embodiments, an antisense compound targeted to nucleotides 22018-22037 of SEQ ID NO: 2 is Oligo ID: 414117.

In certain embodiments, a target region is nucleotides 22873-22892 of SEQ ID NO: 2. In certain embodiments, an antisense compound is targeted to nucleotides 22873-22892 of SEQ ID NO: 2. In certain embodiments, an antisense compound targeted to a TGF-beta1 nucleic acid comprises a nucleotide sequence of SEQ ID NO: 155. In certain such embodiments, an antisense compound targeted to nucleotides 22873-22892 of SEQ ID NO: 2 is Oligo ID: 414118.

In certain embodiments, a target region is nucleotides 23348-23367 of SEQ ID NO: 2. In certain embodiments, an antisense compound is targeted to nucleotides 23348-23367 of SEQ ID NO: 2. In certain embodiments, an antisense compound targeted to a TGF-beta1 nucleic acid comprises a nucleotide sequence of SEQ ID NO: 158. In certain such embodiments, an antisense compound targeted to nucleotides 23348-23367 of SEQ ID NO: 2 is Oligo ID: 414121.

In certain embodiments, the compound or oligonucleotide is modified. In certain embodiments, the oligonucleotide is un-modified. In certain embodiments, the compound is single-stranded. In certain embodiments the compound or oligonucleotide is double stranded. In certain embodiments, the compound or oligonucleotide is 20 linked nucleosides in length.

In certain embodiments, the nucleobase sequence of the compound or oligonucleotide is 90%, 95% or 100% complementary to a nucleobase sequence of SEQ ID NO: 1, SEQ ID NO: 2, or SEQ ID NO: 3.

In certain embodiments, the compound or oligonucleotide has at least one modified internucleoside linkage. In certain embodiments, the internucleoside linkage is a phosphorothioate internucleoside linkage. In certain embodiments, all the internucleoside linkages are phosphorothioate internucleoside linkages.

In certain embodiments, the compound or oligonucleotide has at least one nucleoside comprising a modified sugar. In certain embodiments, at least one modified sugar is a bicyclic or LNA sugar. In certain embodiments, the bicyclic sugar comprises a 4′-CH(CH3)-O-2′ bridge. In certain embodiments, at least one modified sugar comprises a 2′-O-methoxyethyl modification. In certain embodiments, the compound or oligonucleotide has at least one nucleoside comprising a sugar surrogate, as provided herein.

In certain embodiments, the compound or oligonucleotide has at least one modified nucleoside. In certain embodiments, the modified nucleoside is a tetrahydropyran modified nucleoside wherein a tetrahydropyran ring replaces the furanose ring. In certain embodiments, the tetrahydropyran modified nucleoside has the structure:

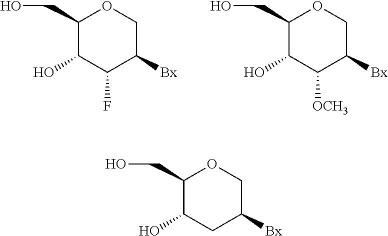

wherein Bx is an optionally protected heterocyclic base moiety. In certain embodiments, each of the at least one tetrahydropyran modified nucleoside has the structure shown above.

In certain embodiments, the compound or oligonucleotide has at least one nucleoside comprising a modified nucleobase. In certain embodiments, the compound or oligonucleotide is un-modified. In certain embodiments, the modified nucleobase is a 5-methylcytosine.

In certain embodiments, the compound or oligonucleotide is chimeric. In certain embodiments, the compound or oligonucleotide is a gapmer.

In certain embodiments, the compound or oligonucleotide has a gap segment of linked deoxynucleosides, a 5′ wing segment of linked nucleosides and a 3′ wing segment of linked nucleosides, wherein the gap segment is positioned immediately adjacent to and between the 5′ wing segment and the 3′ wing segment and wherein each nucleoside of each wing segment has a modified sugar or sugar surrogate. In certain embodiments, each nucleoside of each wing segment has a 2′-O-methoxyethyl sugar modification. In certain embodiments, each internucleoside linkage is a phosphorothioate internucleoside linkage. In certain embodiments, each cytosine is a 5-methylcytosine.

In certain embodiments, the compounds or oligonucleotides provided herein have a gap segment of ten to sixteen linked deoxynucleosides; a 5′ wing segment of two to five linked nucleosides and a 3′ wing segment of two to five linked nucleosides, wherein the gap segment is positioned immediately adjacent to and between the 5′ wing segment and the 3′ wing segment, and wherein each nucleoside of each wing segment has a modified sugar or sugar surrogate. In certain embodiments, each nucleoside of each wing segment has a 2′-O-methoxyethyl sugar modification. In certain embodiments, each internucleoside linkage is a phosphorothioate internucleoside linkage. In certain embodiments, each cytosine is a 5-methylcytosine.

In certain embodiments, the oligonucleotides or compounds provided herein have a gap segment of thirteen linked deoxynucleosides, a 5′ wing segment having two linked nucleosides, and a 3′ wing segment having five linked nucleosides, wherein the gap segment is positioned immediately adjacent to and between the 5′ wing segment and the 3′ wing segment, and wherein each nucleoside of each wing segment has a modified sugar or sugar surrogate. In certain embodiments, each nucleoside of each wing segment has a 2′-O-methoxyethyl sugar modification. In certain embodiments, each internucleoside linkage is a phosphorothioate internucleoside linkage. In certain embodiments, each cytosine is a 5-methylcytosine.

In certain embodiments, compositions are provided having a compound or oligonucleotide provided herein, or a salt thereof, and a pharmaceutically acceptable carrier or diluent. In certain embodiments, the composition comprises a compound or oligonucleotide, or salt thereof, having 12 to 30 linked nucleosides and having a nucleobase sequence containing a contiguous nucleobase portion of a nucleobase sequence selected from among those recited in SEQ ID NOs: 4-159. In certain embodiments, the portion is at least 8, 10, 12, 13, 14, 15, 16, 17, 18, 19 or 20 contiguous nucleobases of a nucleobase sequence selected from among those recited in SEQ ID NOs: 4-159. In certain embodiments, the composition comprises a compound or oligonucleotide or salt thereof, having 12 to 30 linked nucleosides and having a nucleobase sequence containing a contiguous nucleobase portion that is complementary to an equal length nucleobase portion of a region recited herein.

In certain embodiments, provided herein are kits comprising a TGF-beta1 specific inhibitor, as described herein. In certain embodiments, the kit comprises a second therapeutic agent, as described herein. In certain embodiments, the kit is for treating, preventing, ameliorating or slowing the progression of a TGF-beta1 associated disease, as described herein. The kit as provided herein can further include instructions or labels for using the kit to treat, prevent, ameliorate or slow the progression of a TGF-beta1 associated disease, as described herein.

In certain embodiments, methods are provided comprising administering to an animal a compound, oligonucleotide or composition, as described herein.

In certain embodiments, methods are provided to inhibit or reduce TGF-beta1 mRNA or protein expression in an animal by administering to the animal a compound, oligonucleotide or composition, as described herein.

In certain embodiments, the methods as provided herein include treating a TGF-beta1 associated disease in an animal by administering to the animal a therapeutically effective amount of the compound, oligonucleotide or composition, as described herein. In certain embodiments, methods are provided to treat an animal with a disease or condition associated with TGF-beta1 expression comprising identifying the animal with the disease or condition associated with TGF-beta1 expression and administering to the animal a therapeutically effective amount of the compound, oligonucleotide or composition, as described herein.

In certain embodiments, methods are provided for reducing or preventing scarring or fibrosis comprising administering to an animal a therapeutically effective amount of a compound, oligonucleotide or composition, as described herein. In certain embodiments, the compound, oligonucleotide or composition administered to the animal comprises a TGF-beta1 specific inhibitor, described herein. In certain embodiments, the compound, oligonucleotide or composition administered to the animal is a TGF-beta1 specific inhibitor. In certain embodiments, the compound, oligonucleotide or composition administered to the animal has 12 to 30 linked nucleosides and has a nucleobase sequence comprising a contiguous nucleobase portion of a nucleobase sequence selected from among those recited in SEQ ID NOs: 4-159. In certain embodiments, the compound, oligonucleotide or composition administered to the animal has a nucleobase sequence containing a contiguous nucleobase portion that is complementary to an equal length nucleobase portion of a region recited herein. In certain embodiments, a therapeutically effective amount of the TGF-beta1 specific inhibitor is administered to the animal.

In certain embodiments, the animal is a human.

In certain embodiments, the methods provided herein reduce or prevent scarring or fibrosis. In certain embodiments, skin thickness is measured or reduced. In certain embodiments, collagen is measured or reduced. In certain embodiments expression of Col1α2 is measured or reduced.

In certain embodiments, the methods provided herein comprise co-administering the compound, oligonucleotide or composition and a second therapeutic agent, as described herein. In certain embodiments, the compound, oligonucleotide or composition and the second therapeutic agent are administered concomitantly.

In certain embodiments, methods are provided for the treatment, prevention, amelioration or slowing the progression of diseases, disorders, and conditions associated with TGF-beta1 in an individual in need thereof by administering a TGF-beta1 specific inhibitor, as described herein. In certain embodiments, the administering is local administration. In certain embodiments, the administering is parenteral administration. In certain embodiments, the parenteral administration is any of topical, intradermal, subcutaneous, intraperitoneal, inhalation or intravenous administration.

In certain embodiments, the methods as provided herein include reducing the risk for a TGF-beta1 associated disease or disorder in an animal by administering to the animal a therapeutically effective amount of a TGF-beta1 specific inhibitor, as described herein.

Also contemplated are methods, compounds and compositions for the preparation of a medicament for the treatment, prevention, or amelioration of a disease, disorder, or condition associated with TGF-beta1, as described herein.

In certain embodiments, provided herein is the use of a TGF-beta1 specific inhibitor as described herein in the manufacture of a medicament for treating, preventing, or ameliorating a TGF-beta1 associated disease, as described herein, in a patient.

In certain embodiments, provided is any oligonucleotide, compound or composition described herein for use in preventing, ameliorating or treating an animal having a disease or condition associated with expression of TGF-beta1. In certain embodiments, provided herein is any oligonucleotide, compound or composition described herein for use in preventing, ameliorating or treating scarring, fibrosis or a fibrotic condition. In certain embodiments, the fibrotic condition can be scarring in skin or other tissues (e.g. burns, hypertrophic scarring, skin scarring following injury or surgery, scars associated with cosmetic or plastic surgery, or fine-line scars), keloids, liver fibrosis, pulmonary fibrosis, renal fibrosis, cardiac fibrosis, or restenosis. In certain embodiments, the fibrotic condition can be joint fibrosis (including frozen shoulder syndrome, tendon and peripheral nerve damage), spinal cord damage, coronary bypass, abdominal and peritoneal adhesions (including endometriosis, uterine leiomyomata and fibroids), radial keratotomy and photorefractive keratectomy, retinal reattachment surgery, device mediated fibrosis (m, for example, diabetes), tendon adhesions, Dupuytren contracture, or scleroderma. In certain embodiments, the use is parenteral. In certain embodiments, the use topical, intradermal, subcutaneous, intraperitoneal, by inhalation or intravenous administration.

### Compounds

In certain embodiments, the TGF-beta1 specific compounds provided herein are inhibitory compounds. The TGF-beta1 specific compounds provided herein include, but are not limited to, oligomeric compounds such as oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a TGF-beta1 nucleic acid is 12 to 30 subunits in length. In other words, antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments, the antisense compound is an antisense oligonucleotide, and the linked subunits are nucleotides.

In certain embodiments, a shortened or truncated antisense compound targeted to a TGF-beta1 nucleic acid has a single subunit deleted from the 5′ end (5′ truncation), or alternatively from the 3′ end (3′ truncation). A shortened or truncated antisense compound targeted to a TGF-beta1 nucleic acid can have two or more subunits deleted from the 5′ end, or alternatively can have two or more subunits deleted from the 3′ end, of the antisense compound. In certain embodiments, the deleted nucleosides can be dispersed throughout the antisense compound, for example, in an antisense compound having one or more nucleosides deleted from the 5′ end and one or more nucleosides deleted from the 3′ end. In certain embodiments, a shortened antisense compound targeted to a TGF-beta1 nucleic acid can have one or more subunits deleted from the central portion of the antisense compound.

When a single additional subunit is present in a lengthened antisense compound, the additional subunit can be located at the 5′ or 3′ end or the central portion of the antisense compound. When two or more additional subunits are present, the added subunits can be adjacent to each other, for example, in an antisense compound having two subunits added to the 5′ end (5′ addition), or alternatively to the 3′ end (3′ addition), of the antisense compound or the central portion of the antisense compound. Alternatively, the added subunits can be dispersed throughout the antisense compound, for example, in an antisense compound having one or more subunits added to the 5′ end, one or more subunits added to the 3′ end and/or one or more subunits added to the central portion.

It is possible to increase or decrease the length of an antisense compound, such as an antisense oligonucleotide, and/or introduce mismatch bases without eliminating activity as shown by the examples herein and by others as described in the following publications incorporated by reference in their entirety. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Compound Motifs

In certain embodiments, antisense compounds targeted to a TGF-beta1 nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer, an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can, in some embodiments, include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH2)ₙ-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same; in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. Thus, gapmers of the present invention include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, 2-6-2, 2-13-2, 1-8-2, 2-8-3, 3-10-2, 1-18-2, or 2-18-2.

In certain embodiments, the antisense compound has a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a TGF-beta1 nucleic acid possess a 2-13-5 gapmer motif. In certain embodiments, an antisense compound targeted to a TGF-beta1 nucleic acid has a gap-widened motif.

In certain embodiments, a gap-widened antisense oligonucleotide targeted to a TGF-beta1 nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Embodiments of the present invention provide antisense compounds targeted to a TGF-beta1 nucleic acid. In certain embodiments, the human TGF-beta1 nucleic acid is any of the sequences set forth in GENBANK Accession No. NM_{—}000660.3 (incorporated herein as SEQ ID NO: 1) and GENBANK Accession No. NT 011109.15 truncated from 14103000 to 1413000, (incorporated herein as SEQ ID NO: 2). In certain embodiments, the murine TGF-beta1 nucleic acid is the sequence set forth in GENBANK Accession No. NT_{—}039413.7 truncated at nucleotides 23471000 to 23492000 (incorporated herein as SEQ ID NO: 3).

It is understood that the sequence set forth in each SEQ ID NO in the Examples contained herein is independent of any modification to a sugar moiety, an internucleoside linkage, or a nucleobase. As such, antisense compounds defined by a SEQ ID NO can comprise, independently, one or more modifications to a sugar moiety, an internucleoside linkage, or a nucleobase. Antisense compounds described by Oligo ID Number (Oligo ID) indicate a combination of nucleobase sequence and motif.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for TGF-beta1 can be obtained by accession numbers from sequence databases, such as NCBI, and such information is incorporated herein by reference. In certain embodiments, a target region can encompass the sequence from a 5′ target site of one target segment within the target region to a 3′ target site of another target segment within the target region.

In certain embodiments, a “target segment” is a smaller, sub-portion of a target region within a nucleic acid. For example, a target segment can be the sequence of nucleotides of a target nucleic acid to which one or more antisense compounds are targeted. “5′ target site” refers to the 5′-most nucleotide of a target segment. “3′ target site” refers to the 3′-most nucleotide of a target segment.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites listed herein and an ending nucleic acid that is any of the 3′ target sites listed herein.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense compound sequences that can hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in TGF-beta1 mRNA levels are indicative of inhibition of TGF-beta1 expression.

### Hybridization

In some embodiments, hybridization occurs between an antisense compound disclosed herein and a TGF-beta1 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrooke and Russell, Molecular Cloning: A Laboratory Manual, 3^{rd} Ed., 2001). In certain embodiments, the antisense compounds provided herein are specifically hybridizable with a TGF-beta1 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a TGF-beta1 nucleic acid).

Non-complementary nucleobases between an antisense compound and a TGF-beta1 nucleic acid can be tolerated provided that the antisense compound remains able to specifically hybridize to a target nucleic acid. Moreover, an antisense compound can hybridize over one or more segments of a TGF-beta1 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 75%, 80%, 85%, 86%, 87%, 88%, 89%, 90%, 91%, 92%, 93%, 94%, 95%, 96%, 97%, 98%, 99%, or 100% complementary to a TGF-beta1 nucleic acid, a target region, target segment, or specified portion thereof. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining non-complementary nucleobases can be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound can be fully complementary to a TGF-beta1 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. ‘Fully complementary’ can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is ‘fully complementary’ to the target sequence if the target sequence has a corresponding 20 nucleobase portion wherein each nucleobase is complementary to the 20 nucleobase portion of the antisense compound. At the same time, the entire 30 nucleobase antisense compound can or cannot be fully complementary to the target sequence, depending on whether the remaining 10 nucleobases of the antisense compound are also complementary to the target sequence.

The location of a non-complementary nucleobase can be at the 5′ end or 3′ end of the antisense compound. Alternatively, the non-complementary nucleobase or nucleobases can be at an internal position of the antisense compound. When two or more non-complementary nucleobases are present, they can be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a TGF-beta1 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a TGF-beta1 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least an 8, 9, 10, 11, 12, 13, 14, 15, 16, 17, 18, 19, 20, or more nucleobase portion of a target segment, or a range defined by any two of these values.

### Identity

The antisense compounds provided herein can also have a defined percent identity to a particular nucleotide sequence, SEQ ID NO, or the sequence of a compound represented by a specific Oligo ID number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein, as well as compounds having non-identical bases relative to the antisense compounds provided herein, also are contemplated. The non-identical bases can be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a TGF-beta1 nucleic acid comprise one or more modified internucleoside linkages. In certain embodiments, the antisense compounds are unmodified. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides can impart enhanced nuclease stability, increased binding affinity or some other beneficial biological property to the antisense compounds. In certain embodiments, nucleosides comprise a chemically modified ribofuranose ring moiety. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (scc PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl(R or S), 4′-S, 2′-F, 2′-OCH₃ and 2′-O(CH₂)₂OCH₃ substituent groups. The substituent at the 2′ position can also be selected from allyl, amino, azido, thio, O-allyl, O—C1-C10 alkyl, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl.

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2; 4′-(CH₂)₂—O-2′ (ENA); 4′-C(CH₃)₂—O-2′ (see PCT/US2008/068922); 4′-CH(CH₃) [symbol] -O-2′ and 4′-C [symbol] H(CH₂OCH₃) [symbol] -O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see published U.S. Patent Application US2004-0171570, published Sep. 2, 2004); 4′-CH₂—N(R)—O-2′ (see U.S. Pat. No. 7,427,672, issued on Sep. 23, 2008); 4′-CH₂—CH(CH₃)-2′(see Chattopadhyaya et al., J. Org. Chem., 2009, 74, 118-134) and 4′-CH₂—C [symbol] (═CH₂)-2′ (see PCT/US2008/066154); and wherein R is, independently, H, C1-C12 alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226). Previously, α-L-methyleneoxy(4′-CH₂—O-2′) BNA's have also been incorporated into antisense oligonucleotides that showed antisense activity (Frieden et al., Nucleic Acids Research, 2003, 21, 6365-6372).

Further reports related to bicyclic nucleosides can be found in published literature (see for example: Srivastava et al., J. Am. Chem. Soc., 2007, 129, 8362-8379; U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl(C(═O)—H), substituted acyl, CN, sulfonyl (S(═O)₂-J₁), or sulfoxyl(S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl(C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′,4′-(CH₂)₂-2′,4′-(CH₂)₃-2′,4′-CH₂—O-2′,4′-(CH₂)₂—O-2′,4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides include, but are not limited to, (A) α-L-Methyleneoxy(4′-CH₂—O-2′) BNA, (B) β-D-Methyleneoxy(4′-CH₂—O-2′) BNA, (C) Ethyleneoxy(4′-(CH₂)₂—O-2′) BNA, (D) Aminooxy(4′-CH₂—O—N(R)-2′) BNA, (E) Oxyamino(4′-CH₂—N(R)—O-2′) BNA, and (F) Methyl(methyleneoxy)(4′-CH(CH₃)—O-2′) BNA, (G) Methylene-thio(4′-CH₂—S-2′) BNA, (H) Methylene-amino(4′-CH₂—N(R)-2′) BNA, (I) Methyl carbocyclic(4′-CH₂—CH(CH₃)-2′) BNA, and (J) Propylene carbocyclic(4′-(CH₂)₃-2′) BNA as depicted below.

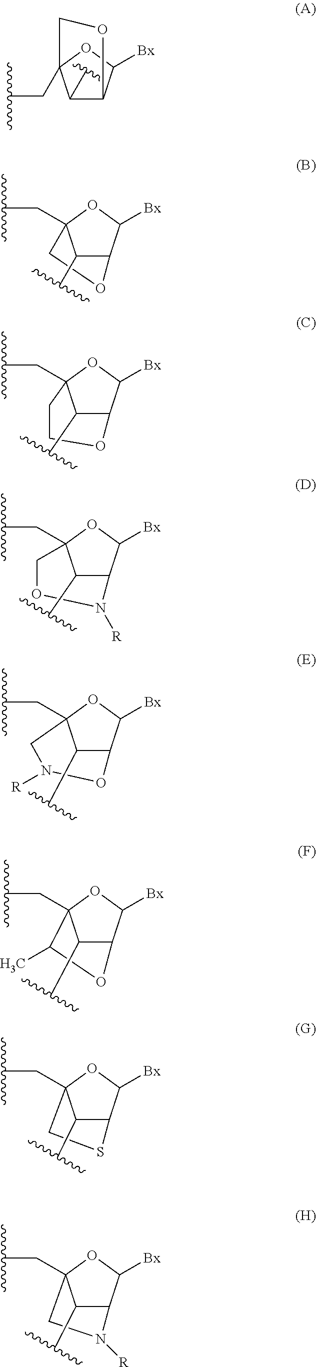

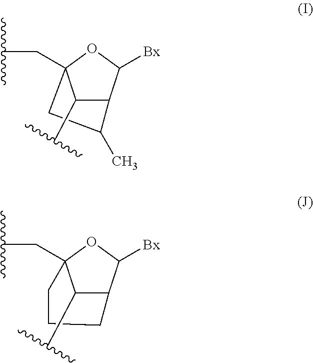

wherein Bx is the base moiety and R is independently H, a protecting group or C₁-C₁₂ alkyl.

In certain embodiments, bicyclic nucleoside having Formula I:

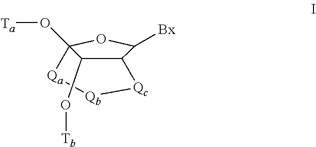

wherein:

Bx is a heterocyclic base moiety;

-Qa-Qb-Qc- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleoside having Formula II:

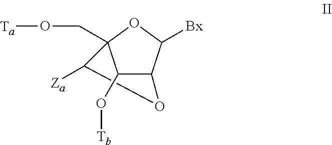

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or M_{c}.

In certain embodiments, bicyclic nucleoside having Formula III:

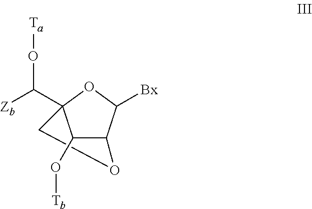

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl(C(═O)—).

In certain embodiments, bicyclic nucleoside having Formula IV:

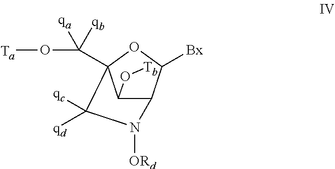

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleoside having Formula V:

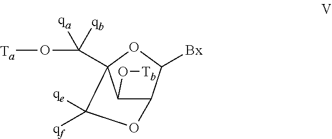

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(q_{b});

q_{g} and qₕ are each, independently, H, halogen, C₁-C₁₂ alkyl or substituted C₁-C₁₂ alkyl.

The synthesis and preparation of the methyleneoxy(4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy(4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

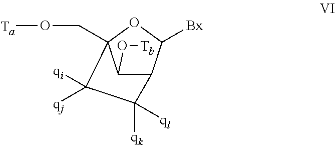

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

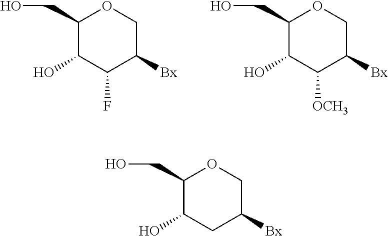

Many other bicyclo and tricyclo sugar surrogate ring systems are also known in the art that can be used to modify nucleosides for incorporation into antisense compounds (see for example review article: Leumann, Christian J., Bioorganic & Medicinal Chemistry, 2002, 10, 841-854). Such ring systems can undergo various additional substitutions to enhance activity. See for example compounds having Formula VII:

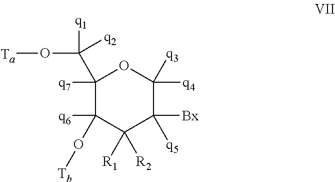

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, subsitituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, qₛ, q₆ and q₇ are each H (M). In certain embodiments, at least one of q₁, q₂, q₃, q₄, qₛ, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro (K). In certain embodiments, THP nucleosides of Formula VII are provided wherein one of R₁ and R₂ is methoxyethoxy. In certain embodiments, R₁ is fluoro and R₂ is H; R₁ is H and R₂ is fluoro; R₁ is methoxy and R₂ is H, and R₁ is H and R₂ is methoxyethoxy. Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a TGF-beta1 nucleic acid comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a bicyclic nucleoside having a (4′-CH(CH₃)—O-2′) bridging group. In certain embodiments, the (4% CH(CH₃)—O-2′) modified nucleotides are arranged throughout the wings of a gapmer motif.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a TGF-beta1 nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a TGF-beta1 nucleic acid comprise one or more modified nucleobases. In certain embodiments, gap-widened antisense oligonucleotides targeted to a TGF-beta1 nucleic acid comprise one or more modified nucleobases. In certain embodiments, the modified nucleobase is 5-methylcytosine. In certain embodiments, each cytosine is a 5-methylcytosine.

### Certain Combination Therapies

The invention also provides methods of combination therapy, wherein, compounds or compositions targeting TGF-beta1 described herein (a first agent) and one or more other therapeutic/prophylactic agents (a second agent, a third agent, et seq.) are administered to treat a condition and/or disease state as described herein.

In certain embodiments, such one or more other therapeutic/prophylactic agents can be another compound or composition targeting TGF-beta1 or can target another molecule. For example, suitable therapeutic/prophylactic compounds include, but are not limited to, antisense oligonucleotides targeting TGF-beta1, CTGF or Smad3, anti-TGF-beta antibodies and TGF-beta receptor inhibitors.

In certain embodiments, such one or more other therapeutic/prophylactic agents are designed to treat the same disease or condition as the compound or composition targeting TGF-beta1. In certain embodiments, such one or more other therapeutic/prophylactic agents are designed to treat a different disease or condition.

In certain embodiments, a compound or composition targeting TGF-beta1 and the therapeutic/prophylactic agents are co-administered as a mixture or administered concomitantly. In certain embodiments, the route of administration is the same for the compound targeting TGF-beta1 and the therapeutic/prophylactic agents, while in other embodiments, the compound or composition targeting TGF-beta1 and the therapeutic/prophylactic agents are administered by different routes. In one embodiment, the dosages of the compound or composition targeting TGF-beta1 and the therapeutic/prophylactic agents are amounts that are therapeutically or prophylactically effective for each compound when administered as independent therapy. Alternatively, the combined administration permits use of lower dosages than would be required to achieve a therapeutic or prophylactic effect if administered as independent therapy. In certain embodiments, combination therapy methods are useful in decreasing one or more side effects of either the TGF-beta1 targeting compound or other agent.

In certain embodiments, a compound or composition targeting TGF-beta1 and one or more other therapeutic/prophylactic agents are administered at the same time. In certain embodiments, a compound or composition compound targeting TGF-beta1 and one or more other therapeutic/prophylactic agents are administered at different times. In certain embodiments, a compound or composition targeting TGF-beta1 and one or more other therapeutic/prophylactic agents are prepared together in a single formulation. In certain embodiments, a compound or composition targeting TGF-beta1 and one or more other therapeutic/prophylactic agents are prepared separately. In certain embodiments, an additive or synergistic effect is achieved by administering a compound or composition targeting TGF-beta1 and one or more other suitable therapeutic/prophylactic agents.

In certain embodiments, the first agent is an antisense compound targeted to TGF-beta1. In some embodiments, the second compound is an antisense compound also targeted to TGF-beta1. In some embodiments, the second compound is an antisense compound not targeted to TGF-beta1.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of TGF-beta1 or the prevention, reduction, amelioration or slowing the progression of a disease or condition associated with TGF-beta1.

In certain embodiments, the variables of the dosing regimen are adjusted to result in a desired concentration of pharmaceutical composition in a subject. “Concentration of pharmaceutical composition” as used with regard to dose regimen can refer to the compound, oligonucleotide, or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from 0.01 μg to 100 mg per kg of body weight, or within a range of 0.001 mg-100 mg intradermal dosing, and may be given once or more daily, weekly, monthly or yearly, or even once every 2 to 20 years. Following successful treatment, it may be desirable to have the patient undergo maintenance therapy to prevent the recurrence of the disease state, wherein the oligonucleotide is administered in maintenance doses, ranging from 0.01 μg to 100 mg per kg of body weight, once or more daily, to once every 20 years, or ranging from 0.001 mg to 100 mg intradermal dosing.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a TGF-beta1 nucleic acid can be utilized in pharmaceutical compositions by combining the antisense compound with a suitable pharmaceutically acceptable diluent or carrier.

In certain embodiments, the “pharmaceutical carrier” or “excipient” is a pharmaceutically acceptable solvent, suspending agent or any other pharmacologically inert vehicle for delivering one or more nucleic acids to an animal. The excipient can be liquid or solid and can be selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients, which do not deleteriously react with nucleic acids, suitable for parenteral or non-parenteral administration can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

A pharmaceutically acceptable diluent includes phosphate-buffered saline (PBS) or sterile water. PBS is a diluent suitable for use in compositions to be delivered parenterally. Accordingly, in one embodiment, employed in the methods described herein is a pharmaceutical composition comprising an antisense compound targeted to a TGF-beta1 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or an oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Administration

The compounds or pharmaceutical compositions of the present invention can be administered in a number of ways depending upon whether local or systemic treatment is desired and upon the area to be treated. Administration can be topical (including ophthalmic and to mucous membranes including vaginal and rectal delivery), intradermal (for local treatment of skin fibrosis or scarring), pulmonary, e.g., by local inhalation or insufflation of powders or aerosols, including by nebulizer; intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration.

In certain embodiments, formulations for topical administration of the compounds or compositions of the invention can include, but is not limited to, pharmaceutical carriers, excipients, sterile and non-sterile aqueous solutions, non-aqueous solutions in common solvents such as alcohols, or solutions of the compounds or compositions in liquid or solid oil bases. The solutions can also contain buffers, diluents and other suitable additives. Formulations for topical administration can include transdermal patches, ointments, lotions, creams, gels, drops, suppositories, sprays, liquids and powders.

In certain embodiments, formulations for oral administration of the compounds or compositions of the invention can include, but is not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, oral formulations are those in which compounds of the invention are administered in conjunction with one or more penetration enhancers, surfactants and chelators.

In certain embodiments, formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

### Indications

In certain embodiments, the invention provides a method of treating a disease or condition associated with expression of TGF-beta1. In certain embodiments, the condition or disease can be a hyperproliferative disorder which includes cancer, a fibrotic condition due to disease, genetic predisposition or injury (e.g., a wound or burn), or scleroderma. In certain embodiments, the cancer can be of the blood, liver, lung, breast, colon, kidney, skin or brain. In certain embodiments, the fibrotic condition can be scarring in skin or other tissues (e.g. burns, hypertrophic scarring, skin scarring following injury or surgery, scars associated with cosmetic or plastic surgery, or fine-line scars), keloids, liver fibrosis, pulmonary fibrosis, renal fibrosis, cardiac fibrosis, or restenosis. In certain embodiments, the disease or condition can be joint fibrosis (including frozen shoulder syndrome, tendon and peripheral nerve damage), spinal cord damage, coronary bypass, abdominal and peritoneal adhesions (including endometriosis, uterine leiomyomata and fibroids), radial keratotomy and photorefractive keratectomy, retinal reattachment surgery, device mediated fibrosis (m, for example, diabetes), tendon adhesions, Dupuytren contracture, or scleroderma.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acids from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of TGF-beta1 nucleic acids can be tested in vitro in a variety of cell types. Cell types used for such analyses are available from commerical vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE2000® (Invitrogen, Carlsbad, Calif.). Antisense oligonucleotide is mixed with LIPOFECTAMINE2000® in OPTI-MEM® 1 reduced serum medium (Invitrogen, Carlsbad, Calif.) to achieve the desired concentration of antisense oligonucleotide and a LIPOFECTAMINE2000® concentration that typically ranges 2 to 12 ug/mL per 100 nM antisense oligonucleotide.

Another reagent used to introduce antisense oligonucleotides into cultured cells includes Oligofectamine™ (Invitrogen Life Technologies, Carlsbad, Calif.). Antisense oligonucleotide is mixed with Oligofectamine™ in Opti-MEM™-1 reduced serum medium (Invitrogen Life Technologies, Carlsbad, Calif.) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, Ind.). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM. Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein (Sambrooke and Russell in Molecular Cloning. A Laboratory Manual. Third Edition. Cold Spring Harbor laboratory Press, Cold Spring Harbor, N.Y. 2001). In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE2000®. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art (Sambrooke and Russell in Molecular Cloning. A Laboratory Manual. Third Edition. Cold Spring Harbor laboratory Press, Cold Spring Harbor, New York. 2001). RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a TGF-beta1 nucleic acid can be assayed in a variety of ways known in the art (Sambrooke and Russell in Molecular Cloning. A Laboratory Manual. Third Edition. Cold Spring Harbor laboratory Press, Cold Spring Harbor, N.Y. 2001). For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels can be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT and real-time-PCR reactions are carried out by methods well known to those skilled in the art.

Gene (or RNA) target quantities obtained by real time PCR can be normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invitrogen, Inc. Carlsbad, Calif.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a TGF-beta1 nucleic acid. Methods for designing real-time PCR probes and primers are well known in the art, and can include the use of software such as PRIMER EXPRESS® Software (Applied Biosystems, Foster City, Calif.).

### In Vivo Testing of Antisense Compounds

Antisense compounds, for example, antisense oligonucleotides, are tested in animals to assess their ability to inhibit expression of TGF-beta1. Testing can be performed in normal animals, or in experimental disease models. For administration to animals, antisense oligonucleotides are formulated in a pharmaceutically acceptable diluent, such as phosphate-buffered saline. Administration includes parenteral routes of administration, such as topical, intraperitoneal, intravenous, and subcutaneous. Calculation of antisense oligonucleotide dosage and dosing frequency depends upon factors such as route of administration and animal body weight. Following a period of treatment with antisense oligonucleotides, RNA is isolated from liver tissue and changes in TGF-beta1 nucleic acid expression are measured.

### Certain Compounds

Provided herein are antisense compounds with improved characteristics. About 157 newly designed antisense compounds were tested for their effect on human TGF-beta1 mRNA in vitro in several cell types. Of the about 157 newly designed antisense compounds, ten compounds were selected for dose response studies based on in vitro potency at single dose 10 nm concentration (Oligo ID NOs 413970, 413979, 413982, 414022, 414035, 414036, 414037, 414040, 414058 and 414102). These compounds effected at least about 80% inhibition of TGF-beta1 in vitro (see Examples 1 and 2). Dose response data further demonstrate (see e.g., Example 3) that the compounds are highly potent, all with IC₅₀ values less than 2 nM and most with IC₅₀ values of less than 1 nM. Therefore, in certain embodiments, the compounds provided herein have IC₅₀ of about or less than about or less than 2 nM, 1.75 nM, 1.5 nM, 1.25 nM, or 1 nM.

From the initial dose response studies, four compounds (Oligo ID NOs 413982, 414035, 414036 and 414040) were selected as being highly potent (IC₅₀s of 0.01 to 0.23 nM; Example 3) and were further tested in confirmatory dose response studies. The confirmatory dose response studies included previously designed compounds including, for example, Oligo IDs 104992 and 113849 which had been determined to be potent antisense compounds in vitro in a previous study to identify potent antisense inhibitors for this gene (see e.g., U.S. Pat. No. 6,436,909). Confirmatory dose response data further demonstrate that the four selected compounds are more potent and more efficacious in reducing TGF-beta 1 expression than previously designed compounds (see Examples 4 and 5). Therefore, in certain embodiments, the compounds provided herein have IC₅₀ of about or less than about 0.25 nM, 0.23 nM, 0.20 nM, 0.175 nM, 0.15 nM, 0.125 nM, 0.1 nM, 0.075 nM, 0.05 nM, 0.025 nM or 0.01 nM.

The four selected compounds were also tested in systemic tolerability studies and compared to Oligo ID 105204, a previously designed benchmark oligo used for in vivo mouse studies (see e.g., U.S. Pat. No. 6,436,909). Three compounds demonstrated improved tolerability (Oligo ID NOs 413982, 414035 and 414036) compared to 105204.

By virtue of their complementarity, the compounds represented by Oligo ID NOs 413970, 413979, 413982, 414022, 414035, 414036, 414037, 414040, 414058 and 414102 (having the nucleobase sequences as set forth in SEQ ID NOs 7, 16, 19, 58, 71, 72, 73, 76, 95, and 139 respectively) are targeted to or are specifically hybridizable with the regions 159-178, 292-317, 1139-1158, 21112134, or 2157-2176 of SEQ ID NO: 1 and/or regions 6452-6471 or 18184-18203 of SEQ ID NO:2, as reported in Tables 1 and 2.

In certain embodiments, the compounds as described herein are efficacious and improved over previously designed compounds by virtue of having at least one of an in vitro IC₅₀ of less than 2 nM, 1.75 nM, 1.6 nM, 1.5 nM, 1.25 nM, 1.00 nM, 0.75 nM, 0.5 nM, 0.4 nM, 0.3 nM, 0.25 nM, 0.20 nM, 0.175 nM, 0.15 nM, 0.1 nM, or 0.05 nM when delivered to HuVEC cells, as described herein. In certain embodiments, the compounds as described herein are highly tolerable in vivo as demonstrated by having minimal increase in either ALT or AST levels of no more than 15 fold, 12 fold, 10 fold, 9 fold, 8 fold, 7 fold, 6 fold, 5 fold, 4 fold, 3 fold, or 2 fold over saline-treated animals even at high doses, for example, at 25 mg/kg or 50 mg/kg delivered by injection twice a week for four weeks. In contrast, certain other new compounds, e.g., Oligo ID NO: 414040 resulted in over a 160 fold increase in ALT compared to placebo controls. In certain embodiments, the compounds as described herein are highly tolerable, as demonstrated by having at least one of an increase in liver, spleen or kidney weight of no more than 40%, 35%, 30%, 25, 20%, 15%, 12%, 10%, 5% or 2% over saline treated animals. In certain embodiments, the compounds as described herein are efficacious and improved over previously designed compounds, by virtue of having any two or more properties described above.

### Certain Indications

In certain embodiments, the invention provides methods of treating an individual comprising administering one or more compounds or pharmaceutical compositions of the present invention. In certain embodiments, the individual has a TGF-beta1 associated disease. In certain embodiments the invention provides methods for prophylactically reducing TGF-beta1 expression in an individual. Certain embodiments include treating an individual in need thereof by administering to an individual a therapeutically effective amount of an antisense compound targeted to a TGF-beta1 nucleic acid.

In one embodiment, administration of a therapeutically effective amount of an antisense compound targeted to a TGF-beta1 nucleic acid is accompanied by monitoring of TGF-beta1 levels or markers of scarring or fibrosis or other disease process associated with the expression of TGF-beta1, to determine an individual's response to administration of the antisense compound. An individual's response to administration of the antisense compound is used by a physician to determine the amount and duration of therapeutic intervention.

In certain embodiments, administration of an antisense compound targeted to a TGF-beta1 nucleic acid results in reduction of TGF-beta1 expression by at least 15, 20, 25, 30, 35, 40, 45, 50, 55, 60, 65, 70, 75, 80, 85, 90, 95 or 99%, or a range defined by any two of these values. In certain embodiments, the reduction is achieved by one or more compounds having a nucleobase sequence or portion of a nucleobase sequence of those recited in SEQ ID NOs 4-159.

In certain embodiments, pharmaceutical compositions comprising an antisense compound targeted to TGF-beta1 are used for the preparation of a medicament for treating a patient suffering or susceptible to a TGF-beta1 associated disease.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Oligonucleotide Sequence Design and Specificity for TGF-Beta1

Multiple specificity steps were incorporated into the discovery of compounds provided herein. For example, Oligo ID Nos. 413982, 414035, 414058, 414037 and 414036 target both human and rhesus monkey TGF-beta1 mRNA sequences, which allow more detailed pharmacology and toxicology studies to be conducted in this latter species. The cross-hybridization design of the ASOs allows for toxicology studies to investigate “on-target” toxicities in primates as well as “off-target” toxicities with the same ASO that may enter human clinical testing. In addition, Oligo ID No. 414036 was designed to hybridize to human, rhesus monkey, rat and mouse. This improved ASO design allows for pharmacology and toxicology studies in all of these species, a major improvement in TGF-beta1 oligonucleotide design.

Numerous sequences highly specific for human TGF-beta1 have been designed such that they do not cross-react (do not have significant complementarity to unrelated gene targets), and hence are not likely to inhibit other unrelated gene targets. This selective design provides an additional safeguard against “off-target” effects that may occur by inhibiting other cross-reacting (complementary) mRNAs. For example, Oligo ID NOs 413982, 414035, 414058, 414037 and 414036 were screened against human genome databases for regions of homology to known genes, predicted genes and other non-annotated sequences.

No off-target binding sites are found at the levels of 20, 19 or 18 bases of homology to any of these five ASO sequences. The complete absence of off-target sites with 20, 19 or 18 bases indicates the strong likelihood of no consequential off-target activity. Therefore, these five sequences are highly specific and selective for TGF-beta1.

### Example 2

### Antisense Inhibition of Human Transforming Growth Factor-Beta 1 (TGF-Beta1) in HuVEC Cells

Antisense oligonucleotides targeted to a TGF-beta1 nucleic acid were tested for their effects on TGF-beta1 mRNA in vitro. Cultured HuVEC cells at a density of 5,000 cells per well were transfected using Lipofectamine™ 2000 reagent with 10 nM antisense oligonucleotide for 4 hours. After a recovery period of approximately 24 hours, RNA was isolated from the cells and TGF-beta1 mRNA levels were measured by quantitative real-time PCR. TGF-beta1 mRNA levels were adjusted according to total RNA content, as measured by RIBOGREEN®. Results are presented as percent inhibition of TGF-beta1, relative to untreated control cells.

The chimeric antisense oligonucleotides in Tables 1 and 2 were designed as 2-13-5 MOE gapmers. Antisense molecules with this motif targeting TGF-beta1 are unique, and represent a novel chemical structure for an ASO directed against this target. The gapmers are 20 nucleotides in length, wherein the central gap segments are comprised of thirteen 2′-deoxynucleotides and are flanked on the 5′ side by wings comprising two nucleotides each and on the 3′ side by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine residues throughout each gapmer are 5-methylcytosines. “Target start site” indicates the 5′-most nucleotide to which the gapmer is targeted. “Target stop site” indicates the 3′-most nucleotide to which the gapmer is targeted. Each gapmer listed in Table 1 is targeted to SEQ ID NO: 1 (GENBANK Accession No. NM_{—}000660.3). Each gapmer listed in Table 2 is targeted to SEQ ID NO: 2 (GENBANK Accession No. NT 011109.15 truncated from 14103000 to 1413000).

The human oligonucleotides also may be cross reactive with the mouse TGF-beta1 genomic sequence (GENBANK Accession No. NT 039413.7 truncated at nucleotides 23471000 to 23492000, incorporated herein as SEQ ID NO: 3), depending on the number of mismatched nucleobases the human oligonucleotide has with the murine TGF-beta1 sequence. “Mouse Target Start Site” indicates the 5′-most nucleotide in the mouse mRNA to which the antisense oligonucleotide is targeted. “Mouse Target Stop Site” indicates the 3′-most nucleotide in the mouse mRNA to which the antisense oligonucleotide is targeted. ‘Mismatches’ indicates the number of nucleobases by which the human oligonucleotide is mismatched with the mouse gene sequence. The designation “n/a” indicates that there was greater than 3 mismatches between the human oligonucleotide and the mouse gene sequence. The greater the complementarity between the human oligonucleotide and the mouse gene sequence, the more likely the human oligonucleotide can cross-react with the mouse gene sequence.

TABLE 1
Inhibition of human TGF-betal mRNA levels by chimeric antisense oligonucleotides
having 2-13-5 MOE wings and deoxy gap targeted to SEQ ID NO: 1

| | Human | Human | | | | Mouse | Mouse | |
|---|---|---|---|---|---|---|---|---|
| | Target | Target | | | SEQ | target | target | |
| Oligo | Start | Stop | | % | ID | start | stop | Mis- |
| ID | Site | Site | Sequence | inhibition | NO | site | site | matches |
| 413967 | 1 | 20 | GATGGCCCAGGGCGCGAAGG | 64 | 4 | 1037 | 1056 | 3 |
| 413968 | 3 | 22 | GAGATGGCCCAGGGCGCGAA | 55 | 5 | n/a | n/a | n/a |
| 413969 | 140 | 159 | CGACTCCTTCCTCCGCTCCG | 53 | 6 | n/a | n/a | n/a |
| 413970 | 159 | 178 | GCCTCAGGCTGCTCCTCGGC | 87 | 7 | 1190 | 1209 | 3 |
| 413971 | 160 | 179 | GGCCTCAGGCTGCTCCTCGG | 78 | 8 | 1191 | 1210 | 3 |
| 413972 | 236 | 255 | CTCGTCCCTCCTCCCGCTCC | 61 | 9 | 1255 | 1274 | 1 |
| 413973 | 280 | 299 | CAACGGAAAAGTCTCAAAAG | 25 | 10 | 1298 | 1317 | 2 |
| 413974 | 282 | 301 | GGCAACGGAAAAGTCTCAAA | 69 | 11 | 1300 | 1319 | 2 |
| 413975 | 284 | 303 | GCGGCAACGGAAAAGTCTCA | 73 | 12 | 1302 | 1321 | 3 |
| 413976 | 286 | 305 | CAGCGGCAACGGAAAAGTCT | 72 | 13 | 283 | 302 | 3 |
| 413977 | 288 | 307 | CCCAGCGGCAACGGAAAAGT | 57 | 14 | n/a | n/a | n/a |
| 413978 | 290 | 309 | CTCCCAGCGGCAACGGAAAA | 67 | 15 | n/a | n/a | n/a |
| 413979 | 292 | 311 | GGCTCCCAGCGGCAACGGAA | 87 | 16 | n/a | n/a | n/a |
| 413980 | 294 | 313 | CCGGCTCCCAGCGGCAACGG | 74 | 17 | n/a | n/a | n/a |
| 413981 | 296 | 315 | CTCCGGCTCCCAGCGGCAAC | 71 | 18 | n/a | n/a | n/a |
| 413982 | 298 | 317 | GCCTCCGGCTCCCAGCGGCA | 88 | 19 | n/a | n/a | n/a |
| 413983 | 300 | 319 | GCGCCTCCGGCTCCCAGCGG | 78 | 20 | n/a | n/a | n/a |
| 413984 | 302 | 321 | CCGCGCCTCCGGCTCCCAGC | 71 | 21 | n/a | n/a | n/a |
| 413985 | 304 | 323 | CCCCGCGCCTCCGGCTCCCA | 66 | 22 | n/a | n/a | n/a |
| 413986 | 306 | 325 | GTCCCCGCGCCTCCGGCTCC | 69 | 23 | n/a | n/a | n/a |
| 413987 | 308 | 327 | AGGTCCCCGCGCCTCCGGCT | 71 | 24 | n/a | n/a | n/a |
| 413988 | 344 | 363 | AAGTCCTGCCTCCTCGCGGG | 63 | 25 | 1362 | 1381 | 3 |
| 413989 | 371 | 390 | GGCAAAGGGAGGCGGTCTGG | 41 | 26 | n/a | n/a | n/a |
| 413990 | 373 | 392 | GCGGCAAAGGGAGGCGGTCT | 59 | 27 | n/a | n/a | n/a |
| 413991 | 375 | 394 | CGGCGGCAAAGGGAGGCGGT | 69 | 28 | n/a | n/a | n/a |
| 413992 | 377 | 396 | CCCGGCGGCAAAGGGAGGCG | 60 | 29 | n/a | n/a | n/a |
| 413993 | 379 | 398 | TCCCCGGCGGCAAAGGGAGG | 52 | 30 | n/a | n/a | n/a |
| 413994 | 381 | 400 | CGTCCCCGGCGGCAAAGGGA | 67 | 31 | n/a | n/a | n/a |
| 413995 | 446 | 465 | CCCGAGGGCTGGTCCGGAAT | 72 | 32 | 1452 | 1471 | 0 |
| 413996 | 476 | 495 | AAGTCTTTGCGGGAGGCCGG | 51 | 33 | 1482 | 1501 | 2 |
| 413997 | 478 | 497 | AAAAGTCTTTGCGGGAGGCC | 41 | 34 | n/a | n/a | n/a |
| 413998 | 480 | 499 | GGAAAAGTCTTTGCGGGAGG | 13 | 35 | 1486 | 1505 | 3 |
| 413999 | 538 | 557 | GGCTCAGGAGACAGGCCGGG | 72 | 36 | n/a | n/a | n/a |
| 414000 | 558 | 577 | AAGGGTCTAGGATGCGCGGG | 75 | 37 | n/a | n/a | n/a |
| 414001 | 591 | 610 | CAGGTCGGAGAGAGATCCGT | 78 | 38 | n/a | n/a | n/a |
| 414002 | 621 | 640 | GGTGGGTGGTCTTGAATAGG | 80 | 39 | 1630 | 1649 | 3 |
| 414003 | 623 | 642 | AAGGTGGGTGGTCTTGAATA | 67 | 40 | 1632 | 1651 | 3 |
| 414004 | 625 | 644 | AGAAGGTGGGTGGTCTTGAA | 70 | 41 | n/a | n/a | n/a |
| 414005 | 627 | 646 | CCAGAAGGTGGGTGGTCTTG | 77 | 42 | n/a | n/a | n/a |
| 414006 | 629 | 648 | TACCAGAAGGTGGGTGGTCT | 85 | 43 | n/a | n/a | n/a |
| 414007 | 631 | 650 | GGTACCAGAAGGTGGGTGGT | 94 | 44 | n/a | n/a | n/a |
| 414008 | 633 | 652 | CTGGTACCAGAAGGTGGGTG | 89 | 45 | n/a | n/a | n/a |
| 414009 | 635 | 654 | ATCTGGTACCAGAAGGTGGG | 80 | 46 | n/a | n/a | n/a |
| 414010 | 637 | 656 | CGATCTGGTACCAGAAGGTG | 88 | 47 | n/a | n/a | n/a |
| 414011 | 639 | 658 | CGCGATCTGGTACCAGAAGG | 85 | 48 | n/a | n/a | n/a |
| 414012 | 641 | 660 | GGCGCGATCTGGTACCAGAA | 87 | 49 | n/a | n/a | n/a |
| 414013 | 643 | 662 | TGGGCGCGATCTGGTACCAG | 90 | 50 | n/a | n/a | n/a |
| 414014 | 645 | 664 | GATGGGCGCGATCTGGTACC | 89 | 51 | n/a | n/a | n/a |
| 414015 | 649 | 668 | CCTAGATGGGCGCGATCTGG | 83 | 52 | n/a | n/a | n/a |
| 414016 | 651 | 670 | AACCTAGATGGGCGCGATCT | 78 | 53 | n/a | n/a | n/a |
| 414017 | 653 | 672 | ATAACCTAGATGGGCGCGAT | 77 | 54 | n/a | n/a | n/a |
| 414018 | 655 | 674 | AAATAACCTAGATGGGCGCG | 82 | 55 | n/a | n/a | n/a |
| 414019 | 657 | 676 | GGAAATAACCTAGATGGGCG | 78 | 56 | n/a | n/a | n/a |
| 414020 | 792 | 811 | GGAGGCCCCGCCCCTGCAGG | 0 | 57 | 1809 | 1828 | 0 |
| 414022 | 1139 | 1158 | GGGCTCCGGTTCTGCACTCT | 84 | 58 | n/a | n/a | n/a |
| 414023 | 1141 | 1160 | TCGGGCTCCGGTTCTGCACT | 78 | 59 | n/a | n/a | n/a |
| 414024 | 1143 | 1162 | GCTCGGGCTCCGGTTCTGCA | 83 | 60 | n/a | n/a | n/a |
| 414025 | 1145 | 1164 | AGGCTCGGGCTCCGGTTCTG | 79 | 61 | n/a | n/a | n/a |
| 414026 | 1149 | 1168 | CCTCAGGCTCGGGCTCCGGT | 82 | 62 | n/a | n/a | n/a |
| 414027 | 1151 | 1170 | GGCCTCAGGCTCGGGCTCCG | 84 | 63 | n/a | n/a | n/a |
| 414028 | 1188 | 1207 | CCATTAGCACGCGGGTGACC | 75 | 64 | 2209 | 2228 | 0 |
| 414029 | 1268 | 1287 | GAGCTCTGATGTGTTGAAGA | 73 | 65 | n/a | n/a | n/a |
| 414030 | 1507 | 1526 | CTAAGGCGAAAGCCCTCAAT | 60 | 66 | n/a | n/a | n/a |
| 414031 | 1555 | 1574 | ATGTCCACTTGCAGTGTGTT | 66 | 67 | n/a | n/a | n/a |
| 414032 | 1891 | 1910 | GGGTTATGCTGGTTGTACAG | 80 | 68 | 18813 | 18832 | 3 |
| 414033 | 1979 | 1998 | CTCCACCTTGGGCTTGCGGC | 80 | 69 | 18901 | 18920 | 1 |
| 414034 | 2109 | 2128 | CCTTAAATACAGCCCCCATG | 68 | 70 | n/a | n/a | n/a |
| 414035 | 2111 | 2130 | GTCCTTAAATACAGCCCCCA | 89 | 71 | 19025 | 19044 | 2 |
| 414036 | 2113 | 2132 | GTGTCCTTAAATACAGCCCC | 87 | 72 | 19027 | 19046 | 0 |
| 414037 | 2115 | 2134 | GGGTGTCCTTAAATACAGCC | 89 | 73 | 19029 | 19048 | 1 |
| 414038 | 2117 | 2136 | ACGGGTGTCCTTAAATACAG | 85 | 74 | 19031 | 19050 | 2 |
| 414039 | 2119 | 2138 | GCACGGGTGTCCTTAAATAC | 83 | 75 | 19033 | 19052 | 3 |
| 414040 | 2157 | 2176 | CTCTCTCCATCTTTAATGGG | 90 | 76 | n/a | n/a | n/a |
| 414041 | 2173 | 2192 | ACAGAGATCCGCAGTCCTCT | 74 | 77 | n/a | n/a | n/a |
| 414042 | 2184 | 2203 | CGCCCAATGACACAGAGATC | 68 | 78 | 19103 | n/a | 3 |
| 414043 | 2289 | 2308 | CCTTGATGCCGGGCAAAGGA | 64 | 79 | n/a | n/a | n/a |
| 414044 | 2326 | 2345 | ATCTAACTACAGTAGTGTTC | 31 | 80 | n/a | n/a | n/a |

The following sets forth target regions of TGF-beta1 nucleic acids. Also illustrated are examples of antisense compounds targeted to the target regions. It is understood that the sequence set forth in each SEQ ID NO is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Oligo ID Number (Oligo ID) indicate a combination of nucleobase sequence and motif.

The following nucleotide regions of SEQ ID NO: 1, when targeted by antisense oligonucleotides, lead to at least 60% inhibition of the target: 1-20, 159-255, 282-305, 290-363, 375-396, 381-465, 538-676, or 1139-2308.

The following nucleotide regions of SEQ ID NO: 1, when targeted by antisense oligonucleotides, lead to at least 65% inhibition of the target: 159-179, 282-305, 290-327, 375-394, 381-465, 538-676, 1139-1287, or 1555-2203.

The following nucleotide regions of SEQ ID NO: 1, when targeted by antisense oligonucleotides, lead to at least 70% inhibition of the target: 159-179, 284-305, 292-321, 308-327, 446-465, 538-640, 625-676, 1139-1287, or 1891-2192.

The following nucleotide regions of SEQ ID NO: 1, when targeted by antisense oligonucleotides, lead to at least 75% inhibition of the target: 159-179, 292-311, 298-319, 558-640, 627-676, 1139-1207, 1891-1998, or 2111-2176.

The following nucleotide regions of SEQ ID NO: 1, when targeted by antisense oligonucleotides, lead to at least 80% inhibition of the target: 159-178, 292-311, 298-317, 621-640, 629-668, 655-674, 1139-1158, 1143-1162, 1149-1170, 1891-1998, or 2111-2176.

The following nucleotide regions of SEQ ID NO: 1, when targeted by antisense oligonucleotides, lead to at least 85% inhibition of the target: 159-178, 292-311, 298-317, 629-652, 637-664, 2111-2136, or 2157-2176.

The following nucleotide regions of SEQ ID NO: 1, when targeted by antisense oligonucleotides, lead to at least 90% inhibition of the target: 631-650, 643-662, or 2157-2176.

TABLE 2
Inhibition of human TGF-betal mRNA levels by chimeric antisense oligonucleotides
having 2-13-5 MOE wings and deoxy gap targeted to SEQ ID NO: 2

| | Human | Human | | | | Mouse | Mouse | |
|---|---|---|---|---|---|---|---|---|
| | Target | Target | | | SEQ | target | target | |
| Oligo | Start | Stop | | % | ID | start | stop | Mis- |
| ID | Site | Site | Sequence | inhibition | NO | site | site | matches |
| 414021 | 3058 | 3077 | TGTACAGGGCGAGCACGGCC | 74 | 81 | 2113 | 2132 | 3 |
| 414045 | 3267 | 3286 | AGCCAGTTTCTTCTGCCAGT | 80 | 82 | n/a | n/a | n/a |
| 414046 | 3891 | 3910 | GTGAAACACCGAGGACACCT | 67 | 83 | n/a | n/a | n/a |
| 414047 | 4228 | 4247 | CCTGCCCCTTGGTGGAAGCG | 41 | 84 | n/a | n/a | n/a |
| 414048 | 4302 | 4321 | GGTTTCCCCAGCCACCCTGA | 61 | 85 | n/a | n/a | n/a |
| 414049 | 4474 | 4493 | CTGAGTGGGAGCCCCGCCCG | 53 | 86 | n/a | n/a | n/a |
| 414050 | 4536 | 4555 | TTCCCCAAGGCTCTGAACCA | 82 | 87 | n/a | n/a | n/a |
| 414051 | 4706 | 4725 | GTCAGTGTTAAAGGAACCTC | 36 | 88 | n/a | n/a | n/a |
| 414052 | 4744 | 4763 | ACACATGTGCATTTGTTGGG | 52 | 89 | n/a | n/a | n/a |
| 414053 | 5034 | 5053 | TTGGCCCGGAGGTTACTCAG | 52 | 90 | n/a | n/a | n/a |
| 414054 | 5615 | 5634 | TGAAGTTCATTCTGGGTAGG | 51 | 91 | n/a | n/a | n/a |
| 414055 | 5661 | 5680 | ATTAGTTTTCCACCCTTAAC | 53 | 92 | n/a | n/a | n/a |
| 414056 | 5996 | 6015 | TTATACCCGTTTAATAGATG | 41 | 93 | n/a | n/a | n/a |
| 414057 | 6423 | 6442 | TACACTGGTCACTCAATCAT | 58 | 94 | n/a | n/a | n/a |
| 414058 | 6452 | 6471 | AGGTCAAGCCATGTGGCACC | 81 | 95 | n/a | n/a | n/a |
| 414059 | 6509 | 6528 | CAAGACAGAGTGACTCTAGA | 66 | 96 | n/a | n/a | n/a |
| 414060 | 6613 | 6632 | ACAGCAATAACATTAAGCTC | 31 | 97 | n/a | n/a | n/a |
| 414061 | 6676 | 6695 | TGTGTGACCATGGGCAGTTA | 69 | 98 | n/a | n/a | n/a |
| 414062 | 6747 | 6766 | CCCCTAAAATGCAGAGTAAG | 72 | 99 | n/a | n/a | n/a |
| 414063 | 6818 | 6837 | AAGTCGACTAAGGCTGGCAC | 80 | 100 | n/a | n/a | n/a |
| 414064 | 6914 | 6933 | TGTGACCTTGAGGAAGTGGT | 61 | 101 | n/a | n/a | n/a |
| 414065 | 7392 | 7411 | AAATGAAGGGAGGCGATCAG | 24 | 102 | n/a | n/a | n/a |
| 414066 | 7661 | 7680 | GTGGACCTTGTAACCAGCCG | 80 | 103 | n/a | n/a | n/a |
| 414067 | 8355 | 8374 | TCCTAGGATGCAAAGAGTCT | 71 | 104 | n/a | n/a | n/a |
| 414068 | 9216 | 9235 | TCTGCAACATCCAAAATAGT | 53 | 105 | n/a | n/a | n/a |
| 414069 | 9362 | 9381 | CTATGAGTTAACATTCCCTC | 62 | 106 | n/a | n/a | n/a |
| 414070 | 9874 | 9893 | GACTAATGTTCTATAAACCC | 54 | 107 | n/a | n/a | n/a |
| 414071 | 10262 | 10281 | TAGAAGTCATTTCTAATGAT | 0 | 108 | n/a | n/a | n/a |
| 414072 | 10754 | 10773 | GCCGAAGGTGTTTTCTTGCC | 48 | 109 | n/a | n/a | n/a |
| 414073 | 10908 | 10927 | CTTCCCCAAACAGGCTTCCA | 65 | 110 | n/a | n/a | n/a |
| 414074 | 11184 | 11203 | AAGTGACCCCAGGACAAACA | 24 | 111 | n/a | n/a | n/a |
| 414075 | 11275 | 11294 | GATTAGCCAATCACTCAGGT | 75 | 112 | n/a | n/a | n/a |
| 414076 | 11401 | 11420 | GTTCCCCAGCTACCTAGCCA | 57 | 113 | n/a | n/a | n/a |
| 414077 | 11917 | 11936 | TCCAGGCCTTTGCACAGGCT | 71 | 114 | n/a | n/a | n/a |
| 414078 | 12055 | 12074 | TGGGCAATTATTGAATAAAA | 18 | 115 | n/a | n/a | n/a |
| 414079 | 12119 | 12138 | GTCTTGGTTATCACTATGTC | 62 | 116 | n/a | n/a | n/a |
| 414080 | 12823 | 12842 | TTGACCAAGACAGATGAGCT | 54 | 117 | n/a | n/a | n/a |
| 414081 | 12838 | 12857 | GCTTGGGACTCAGCATTGAC | 49 | 118 | n/a | n/a | n/a |
| 414082 | 13598 | 13617 | GAGAGGGAAGCCAGTCTGAG | 19 | 119 | n/a | n/a | n/a |
| 414083 | 14052 | 14071 | AACCTGGAGCACCTGGTCAG | 42 | 120 | n/a | n/a | n/a |
| 414084 | 14083 | 14102 | TCAGCCCAAGCACAGCAGCA | 75 | 121 | n/a | n/a | n/a |
| 414085 | 14100 | 14119 | CTAAAGGAGACAGATGCTCA | 66 | 122 | n/a | n/a | n/a |
| 414086 | 14879 | 14898 | TTGAATTCCAACAATCACAG | 54 | 123 | n/a | n/a | n/a |
| 414087 | 14893 | 14912 | GTGACCTTCCAACTTTGAAT | 77 | 124 | n/a | n/a | n/a |
| 414088 | 14959 | 14978 | TTCTAGCATTCTAGAATCCC | 66 | 125 | n/a | n/a | n/a |
| 414089 | 14961 | 14980 | AATTCTAGCATTCTAGAATC | 18 | 126 | n/a | n/a | n/a |
| 414090 | 15020 | 15039 | GATTCCAATGTTTCAGCTTT | 80 | 127 | n/a | n/a | n/a |
| 414091 | 15093 | 15112 | GGTATCCACAATTGGCCAGT | 64 | 128 | n/a | n/a | n/a |
| 414092 | 15205 | 15224 | GAGATACCAATATTCTGCTT | 86 | 129 | n/a | n/a | n/a |
| 414093 | 15234 | 15253 | AACATTCCAACACTGAGTTC | 68 | 130 | n/a | n/a | n/a |
| 414094 | 15636 | 15655 | TCAAGAGGTTCAAACTGACA | 67 | 131 | n/a | n/a | n/a |
| 414095 | 15689 | 15708 | AATTCCAGTATGCCAGTATT | 46 | 132 | n/a | n/a | n/a |
| 414096 | 15717 | 15736 | CCAACCTTTGAGGATCTTGG | 74 | 133 | n/a | n/a | n/a |
| 414097 | 15819 | 15838 | GAATCCAACATTTCAGCTTT | 74 | 134 | n/a | n/a | n/a |
| 414098 | 15888 | 15907 | AAGGGAGGAATAAGGTCAGA | 70 | 135 | n/a | n/a | n/a |
| 414099 | 16960 | 16979 | GTAGGCTATTAATAGTTAAG | 37 | 136 | n/a | n/a | n/a |
| 414100 | 18043 | 18062 | TTCCACTCAATGAATGGAAA | 48 | 137 | n/a | n/a | n/a |
| 414101 | 18114 | 18133 | GACAGCAAGACCAACACCTT | 70 | 138 | n/a | n/a | n/a |
| 414102 | 18184 | 18203 | TTTGAACTACATGGGTCCTC | 86 | 139 | n/a | n/a | n/a |
| 414103 | 18953 | 18972 | ATTCAAGTAAGGTCTACACA | 58 | 140 | n/a | n/a | n/a |
| 414104 | 18956 | 18975 | AGGATTCAAGTAAGGTCTAC | 61 | 141 | n/a | n/a | n/a |
| 414105 | 19039 | 19058 | GATATCTAGAGGAATATCTA | 27 | 142 | n/a | n/a | n/a |
| 414106 | 19046 | 19065 | ATTCCTTGATATCTAGAGGA | 74 | 143 | n/a | n/a | n/a |
| 414107 | 19112 | 19131 | TTCAAATGTATCTCTAATTA | 7 | 144 | n/a | n/a | n/a |
| 414108 | 19149 | 19168 | CACATGCAATCCACCGTGTT | 61 | 145 | n/a | n/a | n/a |
| 414109 | 19512 | 19531 | GGCCAATTTCCATTGCATCT | 80 | 146 | n/a | n/a | n/a |
| 414110 | 19885 | 19904 | GAACAAATTTTCCTATGAAA | 14 | 147 | n/a | n/a | n/a |
| 414111 | 20285 | 20304 | TTGAACAAGCCGTCTAGGTG | 81 | 148 | n/a | n/a | n/a |
| 414112 | 20386 | 20405 | GGCAGCATCACCTGGGAACT | 57 | 149 | n/a | n/a | n/a |
| 414113 | 20883 | 20902 | TCTGGGAAAAAGAGTCCTGG | 79 | 150 | n/a | n/a | n/a |
| 414114 | 21114 | 21133 | TTTCCAAGAGCCACAGAAGC | 46 | 151 | n/a | n/a | n/a |
| 414115 | 21878 | 21897 | TTTTCCATAATAAAGGAATT | 18 | 152 | n/a | n/a | n/a |
| 414116 | 21934 | 21953 | CTGGATGAGAGTTTACGGGC | 83 | 153 | n/a | n/a | n/a |
| 414117 | 22018 | 22037 | AGTGCAATACGGTATTGCAG | 81 | 154 | n/a | n/a | n/a |
| 414118 | 22873 | 22892 | AATGCCCAAGTCCTCACCGT | 73 | 155 | n/a | n/a | n/a |
| 414119 | 23222 | 23241 | TGTGCAACAAATGTTTATTG | 43 | 156 | n/a | n/a | n/a |
| 414120 | 23277 | 23296 | CACACCCTGGAACATACAAA | 40 | 157 | n/a | n/a | n/a |
| 414121 | 23348 | 23367 | GCAATGCTTAAGACAAGCCT | 62 | 158 | n/a | n/a | n/a |
| 414122 | 23408 | 23427 | TCACTAACACAGATTAAGCA | 34 | 159 | n/a | n/a | n/a |

The following nucleotide regions of SEQ ID NO: 2, when targeted by antisense oligonucleotides, lead to at least 60% inhibition of the target: 3058-3077, 3267-3286, 3891-3910, 4302-4321, 4536-4555, 6452-6471, 6509-6528, 6676-6695, 6747-6766, 6818-6837, 6914-6933, 7661-7680, 8355-8374, 9362-9381, 10908-10927, 11275-11294, 11917-11936, 12119-12138, 14083-14102, 14100-14119, 14893-14912, 14959-14978, 15020-15039, 15093-15112, 15205-15224, 15234-15253, 15636-15655, 15717-15736, 15819-15838, 15888-15907, 18114-18133, 18184-18203, 18956-18975, 19046-19065, 19149-19168, 19512-19531, 20285-20304, 20883-20902, 21934-21953, 22018-22037, 22873-22892, or 23348-23367.

The following nucleotide regions of SEQ ID NO: 2, when targeted by antisense oligonucleotides, lead to at least 65% inhibition of the target: 3058-3077, 3267-3286, 3891-3910, 4536-4555, 6452-6471, 6509-6528, 6676-6695, 6747-6766, 6818-6837, 7661-7680, 8355-8374, 10908-10927, 11275-11294, 11917-11936, 14083-14102, 14100-14119, 14893-14912, 14959-14978, 15020-15039, 15205-15224, 15234-15253, 15636-15655, 15717-15736, 15819-15838, 15888-15907, 18114-18133, 18184-18203, 19046-19065, 19512-19531, 20285-20304, 20883-20902, 21934-21953, 22018-22037, or 22873-22892.

The following nucleotide regions of SEQ ID NO: 2, when targeted by antisense oligonucleotides, lead to at least 70% inhibition of the target: 3058-3077, 3267-3286, 4536-4555, 6452-6471, 6747-6766, 6818-6837, 7661-7680, 8355-8374, 11275-11294, 11917-11936, 14083-14102, 14893-14912, 15020-15039, 15205-15224, 15717-15736, 15819-15838, 15888-15907, 18114-18133, 18184-18203, 19046-19065, 19512-19531, 20285-20304, 20883-20902, 21934-21953, 22018-22037, or 22873-22892.

The following nucleotide regions of SEQ ID NO: 2, when targeted by antisense oligonucleotides, lead to at least 75% inhibition of the target: 3267-3286, 4536-4555, 6452-6471, 6818-6837, 7661-7680, 11275-11294, 14083-14102, 14893-14912, 15020-15039, 15205-15224, 18184-18203, 19512-19531, 20285-20304, 20883-20902, 21934-21953, or 22018-22037.

The following nucleotide regions of SEQ ID NO: 2, when targeted by antisense oligonucleotides, lead to at least 80% inhibition of the target: 3267-3286, 4536-4555, 6452-6471, 6818-6837, 7661-7680, 15020-15039, 15205-15224, 18184-18203, 19512-19531, 20285-20304, 21934-21953, or 22018-22037.

The following nucleotide regions of SEQ ID NO: 2, when targeted by antisense oligonucleotides, lead to at least 85% inhibition of the target: 15205-15224 or 18184-18203.

The following antisense compounds target a region of a TGF-beta1 nucleic acid and effect 60% inhibition of a TGF-beta1 mRNA: Oligo IDs 413967, 413970, 413971, 413972, 413974, 413975, 413976, 413978, 413979, 413980, 413981, 413982, 413983, 413984, 413985, 413986, 413987, 413988, 413991, 413992, 413994, 413995, 413999, 414000, 414001, 414002, 414003, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414030, 414031, 414032, 414033, 414034, 414035, 414036, 414037, 414038, 414039, 414040, 414041, 414042, 414043, 414045, 414046, 414048, 414050, 414058, 414059, 414061, 414062, 414063, 414064, 414066, 414067, 414069, 414073, 414075, 414077, 414079, 414084, 414085, 414087, 414088, 414090, 414091, 414092, 414093, 414094, 414096, 414097, 414098, 414101, 414102, 414104, 414106, 414108, 414109, 414111, 414113, 414116, 414117, 414118, and 414121.

The following antisense compounds target a region of a TGF-beta1 nucleic acid and effect 65% inhibition of a TGF-beta1 mRNA: Oligo IDs 413970, 413971, 413974, 413975, 413976, 413978, 413979, 413980, 413981, 413982, 413983, 413984, 413985, 413986, 413987, 413991, 413994, 413995, 413999, 414000, 414001, 414002, 414003, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414031, 414032, 414033, 414034, 414035, 414036, 414037, 414038, 414039, 414040, 414041, 414042, 414045, 414046, 414050, 414058, 414059, 414061, 414062, 414063, 414066, 414067, 414073, 414075, 414077, 414084, 414085, 414087, 414088, 414090, 414092, 414093, 414094, 414096, 414097, 414098, 414101, 414102, 414106, 414109, 414111, 414113, 414116, 414117, and 414118.

The following antisense compounds target a region of a TGF-beta1 nucleic acid and effect 70% inhibition of a TGF-beta1 mRNA: Oligo IDs 413970, 413971, 413975, 413976, 413979, 413980, 413981, 413982, 413983, 413984, 413987, 413995, 413999, 414000, 414001, 414002, 414004, 414005, 414006, 414007, 414008, 414009, 414010, 414011, 414012, 414013, 414014, 414015, 414016, 414017, 414018, 414019, 414021, 414022, 414023, 414024, 414025, 414026, 414027, 414028, 414029, 414032, 414033, 414035, 414036, 414037, 414038, 414039, 414040, 414041, 414045, 414050, 414058, 414062, 414063, 414066, 414067, 414075, 414077, 414084, 414087, 414090, 414092, 414096, 414097, 414098, 414101, 414102, 414106, 414109, 414111, 414113, 414116, 414117, and 414118.

The following antisense compounds target a region of a TGF-beta1 nucleic acid and effect 75% inhibition of a TGF-beta1 mRNA: Oligo IDs 413970, 413971, 413979, 413982, 413983, 414000, 414001, 414002, 414005, 414006, 414007, 414008, 414009, 414010, 414011, 414012, 414013, 414014, 414015, 414016, 414017, 414018, 414019, 414022, 414023, 414024, 414025, 414026, 414027, 414028, 414032, 414033, 414035, 414036, 414037, 414038, 414039, 414040, 414045, 414050, 414058, 414063, 414066, 414075, 414084, 414087, 414090, 414092, 414102, 414109, 414111, 414113, 414116, and 414117.

The following antisense compounds target a region of a TGF-beta1 nucleic acid and effect 75% inhibition of a TGF-beta1 mRNA: Oligo IDs 413970, 413971, 413979, 413982, 413983, 414000, 414001, 414002, 414005, 414006, 414007, 414008, 414009, 414010, 414011, 414012, 414013, 414014, 414015, 414016, 414017, 414018, 414019, 414022, 414023, 414024, 414025, 414026, 414027, 414028, 414032, 414033, 414035, 414036, 414037, 414038, 414039, 414040, 414045, 414050, 414058, 414063, 414066, 414075, 414084, 414087, 414090, 414092, 414102, 414109, 414111, 414113, 414116, and 414117.

The following antisense compounds target a region of a TGF-beta1 nucleic acid and effect 80% inhibition of a TGF-beta1 mRNA: Oligo IDs 413970, 413979, 413982, 414002, 414006, 414007, 414008, 414009, 414010, 414011, 414012, 414013, 414014, 414015, 414018, 414022, 414024, 414026, 414027, 414032, 414033, 414035, 414036, 414037, 414038, 414039, 414040, 414045, 414050, 414058, 414063, 414066, 414090, 414092, 414102, 414109, 414111, 414116, and 414117.

The following antisense compounds target a region of a TGF-beta1 nucleic acid and effect 85% inhibition of a TGF-beta1 mRNA: Oligo IDs 413970, 413979, 413982, 414006, 414007, 414008, 414010, 414011, 414012, 414013, 414014, 414035, 414036, 414037, 414038, 414040, 414092, and 414102.

The following antisense compounds target a region of a TGF-beta1 nucleic acid and effect 90% inhibition of a TGF-beta1 mRNA: Oligo IDs 414007, 414013, and 414040.

In addition, the degree of TGF-beta1 inhibition by these antisense compounds is considerably high, given the low concentration of compound being used (10 nM), demonstrating the high efficacy of these compounds.

### Example 3

### Dose-Dependent Antisense Inhibition of Human TGF-Beta1 in HuVEC Cells

Gapmers from Example 1 (see Tables 1 and 2), exhibiting in vitro inhibition of human TGF-beta1, were tested at various doses in HuVEC cells. Cells were plated at a density of 5,000 cells per well and transfected using Lipofectamine™ 2000 reagent with 0.9375 nM, 1.875 nM, 3.75 nM, 7.5 nM, 15 nM, and 30 nM concentrations of antisense oligonucleotide for 4 hours, as specified in Table 3. After a recovery period of approximately 16 hours, RNA was isolated from the cells and TGF-beta1 mRNA levels were measured by quantitative real-time PCR. Human TGF-beta1 primer probe set RTS 2980 (forward sequence CTCTCCGACCTGCCACAGA, SEQ ID NO: 160; reverse sequence AACCTAGATGGGCGCGATCT, SEQ ID NO: 53; probe sequence CCCTATTCAAGACCACCCACCTTCTGGTX, SEQ ID NO: 161) was used to measure mRNA levels. TGF-beta1 mRNA levels were adjusted according to total RNA content, as measured by RIBOGREEN®. Results are presented as percent inhibition of TGF-beta1, relative to untreated control cells. As illustrated in Table 3, TGF-beta1 mRNA levels were reduced in a dose-dependent manner in antisense oligonucleotide treated cells.

TABLE 3
Dose-dependent antisense inhibition of human TGF-beta1 in HuVEC cells via
transfection of antisense oligonucleotides with Lipofectamine ™ 2000

| Oligo | | | | | | | IC₅₀ |
|---|---|---|---|---|---|---|---|
| ID | 0.9375 nM | 1.875 nM | 3.75 nM | 7.5 nM | 15 nM | 30 nM | (nM) |
| 413970 | 48 | 64 | 81 | 91 | 94 | 95 | 0.66 |
| 413979 | 42 | 62 | 80 | 92 | 95 | 94 | 0.88 |
| 413982 | 56 | 77 | 88 | 95 | 96 | 97 | 0.23 |
| 414022 | 31 | 50 | 76 | 85 | 91 | 92 | 1.67 |
| 414035 | 64 | 75 | 88 | 94 | 95 | 94 | 0.10 |
| 414036 | 56 | 75 | 86 | 93 | 95 | 93 | 0.22 |
| 414037 | 56 | 74 | 87 | 93 | 93 | 92 | 0.21 |
| 414040 | 72 | 83 | 90 | 94 | 95 | 95 | 0.01 |
| 414058 | 46 | 65 | 78 | 88 | 87 | 81 | 0.51 |
| 414102 | 36 | 54 | 70 | 84 | 87 | 84 | 1.42 |

These data demonstrate that the ASOs evaluated above are highly potent, all with IC₅₀ values less than 2 nM and most with IC₅₀ values of less than 1 nM. These are much more potent than previously designed 2′MOE-containing ASOs targeting TGF-beta1 described in U.S. Pat. No. 6,436,909. We have formally compared the best ASO sequences from the U.S. Pat. No. 6,436,909 disclosure with the most potent described here in the next example.

### Example 4

### Dose-Dependent Antisense Inhibition of Human TGF-Beta1 in HuVEC Cells

Selected gapmers from Example 2 (see Table 3), exhibiting in vitro inhibition of human TGF-beta1, were tested at various doses in HuVEC cells. The dose-dependent antisense inhibition potential of these gapmers was compared with that of Oligo IDs 104992 and 1138498 from U.S. Pat. No. 6,436,909. Cells were plated at a density of 5,000 cells per well and transfected using Lipofectamine™ 2000 reagent with 0.3292 nM, 0.9877 nM, 2.963 nM, 8.8889 nM, 26.6667 nM, and 80 nM concentrations of antisense oligonucleotide for 4 hours, as specified in Table 4. After a recovery period of approximately 16 hours, RNA was isolated from the cells and TGF-beta1 mRNA levels were measured by quantitative real-time PCR. Human TGF-beta1 primer probe set RTS 2980 was used to measure mRNA levels. TGF-beta1 mRNA levels were adjusted according to total RNA content, as measured by RIBOGREEN®. Results are presented as percent inhibition of TGF-beta1, relative to untreated control cells. As illustrated in Table 4 and 5, TGF-beta1 mRNA levels were reduced in a dose-dependent manner in antisense oligonucleotide treated cells. These data confirm the high potency of the newly designed compounds compared to previously designed compound, with the new compounds having IC₅₀ values below 0.3 nM (see table 5). All the antisense oligonucleotides in Table 4 target human TGF-beta1 mRNA (SEQ ID NO: 1).

TABLE 4
Dose-dependent antisense inhibition of human TGF-beta1 in HuVEC cells via
transfection of antisense oligonucleotides with Lipofectamine ™ 2000

| | Human | | | | | | |
|---|---|---|---|---|---|---|---|
| Oligo | Start | | | | | | |
| ID | Site | 0.3292 nM | 0.9877 nM | 2.963 nM | 8.8889 nM | 26.6667 nM | 80.0 nM |
| 104992 | 2179 | 26 | 41 | 44 | 45 | 51 | 45 |
| 113849 | 1193 | 55 | 70 | 82 | 88 | 87 | 88 |
| 413982 | 298 | 76 | 87 | 91 | 95 | 95 | 93 |
| 414035 | 2111 | 74 | 84 | 92 | 94 | 93 | 93 |
| 414036 | 2113 | 70 | 83 | 91 | 93 | 92 | 89 |
| 414040 | 2157 | 85 | 90 | 94 | 94 | 94 | 93 |

### Example 5

### Dose-Dependent Confirmation of Antisense Inhibition of Human TGF-Beta1 in HuVEC Cells

Selected gapmers from Example 4 (see Table 4), exhibiting in vitro inhibition of human TGF-beta1, were tested after large-scale synthesis at various doses in HuVEC cells. Cells were plated at a density of 5,000 cells per well and transfected using Lipofectamine™ 2000 reagent with 0.007 nM, 0.021 nM, 0.062 nM, 0.185 nM, 0.556 nM, 1.667 nM, 5 nM, and 15 nM concentrations of antisense oligonucleotide for 4 hours, as specified in Table 5. After a recovery period of approximately 16 hours, RNA was isolated from the cells and TGF-beta1 mRNA levels were measured by quantitative real-time PCR. Human TGF-beta1 primer probe set RTS 2980 was used to measure mRNA levels. TGF-beta1 mRNA levels were adjusted according to total RNA content, as measured by RIBOGREEN®. Results are presented as percent inhibition of TGF-beta1, relative to untreated control cells. As illustrated in Table 5, TGF-beta1 mRNA levels were reduced in a dose-dependent manner in antisense oligonucleotide treated cells. These data confirm the unexpectedly high potency of these molecules with IC₅₀ values below 1 nM.

TABLE 5
Dose-dependent antisense inhibition of human TGF-beta1 in HuVEC cells via
transfection of antisense oligonucleotides with Lipofectamine ™ 2000

| Oligo | | | | | | | | | IC₅₀ |
|---|---|---|---|---|---|---|---|---|---|
| ID. | 0.007 nM | 0.021 nM | 0.062 nM | 0.185 nM | 0.556 nM | 1.667 nM | 5 nM | 15 nM | (nM) |
| 413982 | 0 | 14 | 31 | 56 | 71 | 79 | 89 | 92 | 0.24 |
| 414035 | 9 | 26 | 45 | 59 | 76 | 84 | 90 | 91 | 0.13 |
| 414036 | 5 | 26 | 41 | 58 | 73 | 84 | 91 | 90 | 0.15 |
| 414040 | 19 | 45 | 58 | 76 | 83 | 89 | 92 | 93 | 0.04 |

### Example 6

### Dose-Dependent Confirmation of Antisense Inhibition of Human TGF-Beta1 in HuVEC Cells

The gapmers from Example 5 (see Table 5) were also tested at various doses in HuVEC cells using electroporation as the transfection reagent. Cells were plated at a density of 20,000 cells per well and transfected using electroporation with 0.15625 nM, 0.3125 nM, 0.625 nM, 1.25 nM, 2.5 nM, 5 nM, 10 nM, and 20 nM concentrations of antisense oligonucleotide for 4 hours, as specified in Table 6. After a recovery period of approximately 16 hours, RNA was isolated from the cells and TGF-beta1 mRNA levels were measured by quantitative real-time PCR. TGF-beta1 mRNA levels were adjusted according to total RNA content, as measured by RIBOGREEN®. Results are presented as percent inhibition of TGF-beta1, relative to untreated control cells. As illustrated in Table 6, TGF-beta1 mRNA levels were reduced in a dose-dependent manner in antisense oligonucleotide treated cells. These data confirm the unexpectedly high potency of these molecules.

TABLE 6
Dose-dependent antisense inhibition of human TGF-beta1 in HuVEC
cells via transfection of antisense oligonucleotides with electroporation

| Oligo ID. | 0.15625 nM | 0.3125 nM | 0.625 nM | 1.25 nM | 2.5 nM | 5.00 nM | 10.00 nM | 20 nM |
|---|---|---|---|---|---|---|---|---|
| 413982 | 26 | 32 | 57 | 79 | 85 | 89 | 93 | 96 |
| 414035 | 17 | 49 | 63 | 77 | 89 | 94 | 94 | 94 |
| 414036 | 10 | 38 | 55 | 74 | 83 | 92 | 94 | 92 |
| 414040 | 57 | 68 | 81 | 91 | 93 | 95 | 94 | 94 |

### Example 7

### Tolerability of Human TGF-Beta1 Antisense Oligonucleotides in BALB/C Mice

Gapmers targeted to human TGF-beta1 (Examples 5 and 6, Tables 5 and 6) were further evaluated in vivo in mice. BALB/c mice were treated with Oligo ID Nos. 413982, 414035, 414036, or 414040. These gapmer oligonucleotides were designed to target human TGF-beta1 and have varying degrees of mismatch with murine TGF-beta1 sequence, as shown in Table 1. Oligo ID Nos. 413982 and 414040 have greater than 3 mismatches to the murine TGF-beta1 sequence. Oligo ID No. 414035 has 2 mismatches to murine TGF-beta1. Oligo ID No. 414036 has no mismatches to murine TGF-beta1.

### Treatment

BALB/c mice were injected with 25 mg/kg or 50 mg/kg of Oligo ID Nos. 413982, 414035, 414036, or 414040 twice a week for 4 weeks. A control group of mice was injected with phosphate buffered saline (PBS) twice a week for 4 weeks. Plasma transaminase levels were evaluated bi-weekly.

### Plasma Transaminase Measurement

Elevated levels of plasma transaminases are often used clinically as potential indicators of liver damage. To evaluate the impact of TGF-beta1 antisense oligonucleotides on the hepatic function of mice described above, plasma concentrations of transaminases were measured using an automated clinical chemistry analyzer (Hitachi Olympus AU400e, Melville, N.Y.). Measurements of alanine transaminase (ALT) and aspartate transaminase (AST) were taken after antisense oligonucleotide treatment and are shown in Tables 7 and 8.

TABLE 7
Effect of antisense oligonucleotides on alanine transaminase
levels (IU/L)

| | Mg/kg | Week 2 | Week 4 |
|---|---|---|---|
| PBS | 0 | 38 | 30 |
| Oligo ID | 50 | 77 | 462 |
| 413982 | 25 | 59 | 140 |
| Oligo ID | 50 | 66 | 67 |
| 414035 | 25 | 56 | 45 |
| Oligo ID | 50 | 99 | 190 |
| 414036 | 25 | 70 | 61 |
| Oligo ID | 50 | 837 | 4997 |
| 414040 | 25 | 178 | 2248 |

TABLE 8
Effect of antisense oligonucleotides on aspartate transaminase
levels (IU/L)

| | Mg/kg | Week 2 | Week 4 |
|---|---|---|---|
| PBS | 0 | 71 | 59 |
| Oligo ID | 50 | 92 | 460 |
| 413982 | 25 | 74 | 161 |
| Oligo ID | 50 | 123 | 155 |
| 414035 | 25 | 82 | 116 |
| Oligo ID | 50 | 183 | 467 |
| 414036 | 25 | 119 | 148 |
| Oligo ID | 50 | 529 | 2237 |
| 414040 | 25 | 128 | 1405 |

Dosing mice for four weeks with four ASO molecules (Oligo ID Nos 413982, 414035, 414036 and 414040) targeting human TGF-beta1 demonstrated differences in ALT/AST levels in the mice. Increases in ALT/AST levels may indicate the possibility of liver toxicity. This effect is sequence dependent and is not dependent upon inhibition of TGF-beta1. Oligo ID No. 414035 exhibit less than a 3 fold increase in ALT/AST at these dose levels. Oligo ID Nos: 414036 and 413982 exhibit less than an 8 fold and less than a 16 fold increase, respectively, in ALT/AST at these dose levels. In contrast, Oligo ID NO: 414040 resulted in a 166 fold increase in ALT levels.

### Example 8

### Tolerability of TGF-Beta1 Antisense Oligonucleotides in BALB/C Mice

Oligo ID 105204 (GTCCACCATTAGCACGCGGG, murine target start site 2214, SEQ ID NO: 165), targeted to the murine TGF-beta1 gene sequence (SEQ ID NO: 3) and having one mismatch to human TGF-beta1 mRNA (SEQ ID NO: 1; human target start site 1193), and Oligo ID 414036 targeted to the human TGF-beta1 mRNA (GENBANK Accession No. NM_{—}000660.3, designated herein as SEQ ID NO: 1) were tested in vivo.

### Treatment

BALB/c mice were injected with 25 mg/kg or 50 mg/kg of Oligo ID Nos. 414036 or 105204 twice a week for 4 weeks. A control group of mice was injected with phosphate buffered saline (PBS) twice a week for 4 weeks. The mice were sacrificed 2 days after the last administration and liver, spleen and kidney weights were measured. Plasma transaminase levels were also evaluated.

### Plasma Transaminase Measurement

To evaluate the impact of antisense oligonucleotides on hepatic function of mice described above, plasma concentrations of transaminases were measured using an automated clinical chemistry analyzer (Hitachi Olympus AU400e, Melville, N.Y.). Measurements of alanine transaminase (ALT) and aspartate transaminase (AST) are shown in Table 9.

TABLE 9
Effect of ISIS oligonucleotides on ALT and AST levels (IU/L)

| | Dose | | |
|---|---|---|---|
| | (mg/kg) | ALT | AST |
| PBS | 0 | 31 | 68 |
| 414036 | 50 | 182 | 460 |
| | 25 | 51 | 112 |
| 105204 | 50 | 3169 | 1640 |
| | 25 | 684 | 409 |

### Organ Weights

The weights of liver, kidney and spleen of the mice were measured after 4 weeks and liver weights are presented in Table 10 as a percentage change compared to the corresponding weights in the PBS control. The percentage changes in kidney and spleen weight in the treated mice compared to the PBS control were negligible and are not shown.

TABLE 10
Liver weight change in treated mice compared to the PBS control

| | | % Weight |
|---|---|---|
| | | change |
| Oligo ID | Dose | relative to |
| No. | (mg/kg | PBS control |
| 414036 | 50 | +35 |
| | 25 | +17 |
| 105204 | 50 | +73 |
| | 25 | +28 |

Oligo ID NO: 414036 which inhibits murine TGF-beta1 expression (data not shown), exhibits no more than a 35 fold increase in liver weight at the tested dose levels compared to 105204 which exhibits greater than a 70 fold increase.

### Example 9

### Inhibition of Collagen1α2 Expression by a Rat Antisense Oligonucleotide Targeting TGF-Beta1 in a Rat Model of Skin Fibrosis and Wounding

Scar and fibrotic tissues are mainly composed of collagen, especially collagen1α2 (Col1α2). Therefore, the expression of Col1α2 can be used as a marker for the severity of scarring, especially in skin. We have evaluated the ability of a TGF-beta1 ASO to suppress the expression of Col1α2 in rat skin subsequent to full-thickness skin wounding, an injury that typically leads to a 4-6 fold induction in Col1α2 expression.

### Treatment

On Day 1 of the study, a 0.8 centimeter biopsy punch was used to create full-thickness wounds on the back of anesthetized adult hairless rats. Two biopsies were performed on each rat's back; one in the lower left quadrant, and one in the upper right quadrant. The wounds were left open, but dressed with a sterile occlusive bandage, which were left in place for 24 hours.

Biopsy sites were treated intradermally with PBS (vehicle) or a 3 mg dose of a rat specific TGF-beta1 antisense oligonucleotide (Oligo ID 433849; SEQ ID NO. 166) on Days 1, 5, 9, and 13 post-biopsy. Animals were sacrificed on Day 14 post-biopsy. A total volume of 200 μl of PBS or oligonucleotide solution was delivered to each punch biopsy wound site. The 200 μl volume was divided into four 50 μl aliquots injected at 90 degree intervals around the circumference of the wound, to the upper left, upper right, lower left, and lower right “quadrants” of the wound.

A subset of the excised skin from each initial biopsy site was retained and prepared for Col1α2 mRNA expression (by RT-PCR). This constituted the Day 0 (un-manipulated) skin sample for determining baseline Col1α2 mRNA levels. On day 15, animals were euthanized, a sample of skin from the center of the wound was obtained with a 0.5 cm biopsy punch, and Col1α2 mRNA expression determined.

### RNA Analysis

As presented in Table 11, Col1α2 mRNA expression was induced approximately 5-fold day 14 after skin wounding. Treatment of the skin wounds with a TGF-beta antisense oligonucleotide (Oligo ID 433849) significantly reduced the expression of Col1α2 in rat skin. These data clearly demonstrate that in animals, intradermal administration of a TGF-beta1 antisense oligonucleotide can reduce the severity of skin fibrosis and scarring.

TABLE 11
Effect of antisense inhibition on Col1α2 mRNA compared
to the unwounded control at day 14 after skin wounding

| | % |
|---|---|
| | Col1α2 |
| PBS | 409 |
| Oligo ID | 83 |
| 433849 | |

### Example 10

### Inhibition of Collagen1α2 Expression by a Mouse Antisense Oligonucleotide Targeting TGF-Beta1 in a Bleomycin-Induced Murine Model of Skin Fibrosis

The ability of a TGF-beta1 ASO to reduce the induction of skin fibrosis in a bleomycin-induced model of dermal fibrosis was evaluated.

### Treatment

Two groups of 8 C57BL/6 mice each were treated with bleomycin every other day for 19 days. Bleomycin, at a concentration of 10 mg/mL in PBS and at a volume of 0.1 mL, was injected subcutaneously into the shaved backs of the mice. The injection site was divided into 4 quadrants. The 100 μL volume of bleomycin was divided into four 25 μL aliquots injected at 90 degree intervals, to the upper left, upper right, lower left, and lower right “quadrants”.

Each of the two groups was treated intradermally with PBS (vehicle) or a 5 mg dose of a TGF-beta1 antisense oligonucleotide (Oligo ID 433849) twice a week, starting from day 1 of bleomycin administration. Animals were sacrificed on Day 18 of the study. Skin thickness was measured by skin calipers on 6-mm punch biopsy specimens obtained from the upper back of the mice. Breaking strength of the skin was measured on the 6-mm punch biopsy specimens using a tensiometer (Series EG2 digital force gauge; Mark-10, Copiague, N.Y.), and the point of maximal stress before tearing of the biopsy specimen was recorded. All measurements were undertaken in a blinded manner.

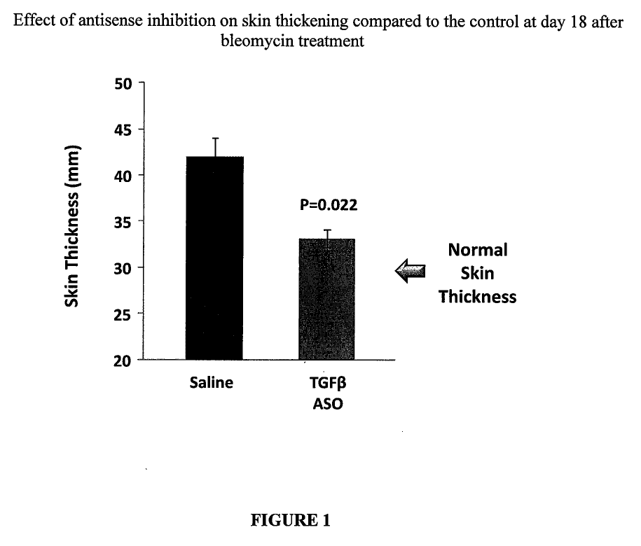

The results of skin thickness measurement are presented in FIG. 1. Treatment of mice with bleomycin resulted in thickening of the skin from 30 mm to 40-45 mm. Treatment of mice with the TGF-beta1 antisense oligonucleotide significantly reduced bleomycin-induced skin fibrosis and thickening.

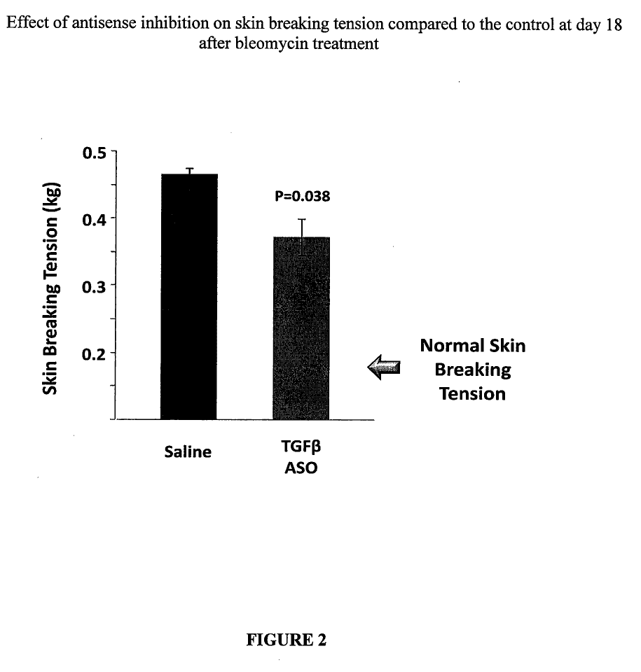

The results of skin breaking strength are presented in FIG. 2. Treatment of mice with bleomycin caused a significant increase in the breaking strength of skin as a result of increased fibrosis and thickening. Breaking strength of untreated skin is typically 0.2 kg tension, which was increased to approximately 0.47 kg by bleomycin treatment. Pre-treatment of the mice with TGF-beta1 ASO significantly reduced the bleomycin-induced fibrosis and skin thickening.

Hence, treatment with a TGF-beta1 ASO reduced the severity of bleomycin-induced skin fibrosis, and thickening.

1. A compound comprising a modified or unmodified oligonucleotide consisting of 12 to 30 contiguous linked nucleosides and having a nucleobase sequence comprising at least 8 contiguous nucleobases of a sequence recited in SEQ ID NOs: 4-159, wherein each nucleoside is linked to any immediately adjacent nucleoside linkage; or a pharmaceutically acceptable salt of such compound.

2. A compound comprising a modified oligonucleotide consisting of 12 to 30 linked nucleosides and having a nucleobase sequence comprising a portion which consists of 8 contiguous nucleobases complementary to an equal-length portion of nucleotides 1-22, 1-20, 140-179, 159-179, 236-255, 280-327, 282-363, 282-305, 290-363, 290-327, 292-321, 371-400, 373-400, 375-396, 381-400, 446-497, 446-495, 446-465, 538-676, 538-640, 558-640, 625-676, 627-676, 629-668, 631-652, 637-664, 1139-1207, 1149-1170, 1139-1170, 2109-2203, 2109-2192, 2109-2176, 2109-2138, 2111-2176, 2111-2138, 2111-2136, 2111-2192, 2157-2203, or 2157-2192 of SEQ ID NO: 1, and wherein the nucleobase sequence of the modified oligonucleotide is at least 90% complementary to SEQ ID NO: 1.

3. (canceled)

4. (canceled)

5. The compound of claim 1, wherein the modified oligonucleotide hybridizes exclusively within nucleotides 1-22, 1-20, 140-179, 159-179, 236-255, 280-327, 282-363, 282-305, 290-363, 290-327, 292-321, 371-400, 373-400, 375-396, 381-400, 446-497, 446-495, 446-465, 538-676, 538-640, 558-640, 625-676, 627-676, 629-668, 631-652, 637-664, 1139-1207, 1149-1170, 1139-1170, 2109-2203, 2109-2192, 2109-2176, 2109-2138, 2111-2176, 2111-2138, 2111-2136, 2111-2192, 2157-2203, or 2157-2192 of SEQ ID NO: 1, and wherein the nucleobase sequence of the modified oligonucleotide is at least 90% complementary to SEQ ID NO: 1.

6. (canceled)

7. (canceled)

8. (canceled)

9. The compound of claim 1 or 2, wherein the oligonucleotide is a single-stranded oligonucleotide.

10. The compound of claim 1, wherein the nucleobase sequence of the modified oligonucleotide is 90%, 95% or 100% complementary to SEQ ID NO 1 or 2.

11. (canceled)

12. (canceled)

13. The compound of claim 1, wherein at least one internucleoside linkage is a modified internucleoside linkage.

14. The compound of claim 13, wherein each internucleoside linkage is a phosphorothioate internucleoside linkage.

15. The compound of claim 1, wherein at least one nucleoside comprises a modified sugar.

16. The compound of claim 15, wherein at least one modified sugar is a bicyclic sugar.

17. The antisense compound of claim 16, wherein each of the at least one bicyclic sugar comprises a 4′-CH(CH3)-O-2′ bridge.

18. The antisense compound of claim 15, wherein at least one modified sugar comprises a 2′-O-methoxyethyl group.

19. The antisense compound of claim 1, comprising at least one tetrahydropyran modified nucleoside wherein a tetrahydropyran ring replaces the furanose ring.

20. The antisense compound of claim 19, wherein each of the at least one tetra-hydropyran modified nucleoside has the structure: 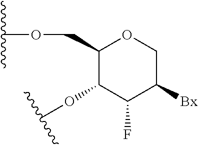
wherein Bx is an optionally protected heterocyclic base moiety.

21. The compound of claim 1, wherein at least one nucleoside comprises a modified nucleobase.

22. The compound of claim 21, wherein the modified nucleobase is a 5-methylcytosine.

23. The compound of claim 1, wherein the modified oligonucleotide comprises:
a gap segment consisting of linked deoxynucleosides;
a 5′ wing segment consisting of linked nucleosides;
a 3′ wing segment consisting of linked nucleosides;
wherein the gap segment is positioned between the 5′ wing segment and the 3′ wing segment and wherein each nucleoside of each wing segment comprises a modified sugar.

24. The compound of claim 23, wherein the modified oligonucleotide comprises:
a gap segment consisting of thirteen linked deoxynucleosides;
a 5′ wing segment consisting of two linked nucleosides;
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; and wherein each internucleoside linkage is a phosphorothioate linkage.

25. The compound of claim 1, wherein the modified oligonucleotide consists of 20 linked nucleosides.

26. A composition comprising the compound of claim 1, or salt thereof, and a pharmaceutically acceptable carrier or diluent.

27. A method comprising administering to an animal the composition of claim 26, wherein administering the composition prevents, treats, ameliorates, or slows progression of a disease or condition associated with TGF-beta1 expression or of a symptom associated therewith.

28. The method of claim 27, wherein the animal is a human.

29. (canceled)

30. The method of claim 27, comprising co-administering the composition and a second agent.

31. The method of claim 30, wherein the composition and the second agent are administered concomitantly.

32. The method of claim 27, wherein the administering is effected by local administration, subcutaneous administration, topical administration and/or intradermal administration.

33. A method to reduce TGF-beta1 mRNA or protein expression in an animal comprising administering to the animal the composition of claim 26 to reduce TGF-beta1 mRNA or protein expression in the animal.

34. The method of claim 33, wherein the animal is a human.

35. The method of claim 33, wherein reducing TGF-beta1 mRNA or protein expression prevents, treats, ameliorates, or slows progression of a disease or condition associated with TGF-beta1 expression.

36. The method of claim 33, comprising co-administering the composition and a second agent.

37. The method of claim 36, wherein the composition and the second agent are administered concomitantly.

38. The method of claim 33, wherein the administering is effected by local administration, subcutaneous administration, topical administration and/or intradermal administration.

39. A method for treating a human with a disease or condition associated with TGF-beta1 expression comprising identifying the human with the disease or condition associated with TGF-beta1 expression and administering to the human a therapeutically effective amount of the composition of claim 26 so as to treat the human for the disease or condition associated with TGF-beta1 expression.

40. The method of claim 39, wherein the treatment reduces or prevents fibrosis.

41. The method of claim 40, wherein the fibrosis is scarring.

42. The method of claim 39, comprising co-administering the composition and a second agent.

43. The method of claim 42, wherein the compound or composition and the second agent are administered concomitantly.

44. The method of claim 39, wherein the administering is effected by local administration, subcutaneous administration, topical administration and/or intradermal administration.

45. A method for reducing or preventing scarring or fibrosis comprising administering to a human a therapeutically effective amount of the composition of claim 26, thereby reducing or preventing scarring or fibrosis.

46. The method of claim 45, comprising co-administering the composition and a second agent.

47. The method of claim 46, wherein the composition and the second agent are administered concomitantly.

48. The method of claim 45, wherein the administering is effected by local administration, subcutaneous administration, topical administration, and/or intradermal administration.

49. A method of reducing or preventing scarring or fibrosis comprising administering by intradermal delivery to an animal a therapeutically effective amount of a compound comprising an oligonucleotide targeting SEQ ID NO 1 or 2, thereby reducing or preventing scarring or fibrosis.


SEQUENCE LISTING (166 sequences)

SEQ ID NO: 1 - DNA, 2346 bp, H. sapiens
ccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatc

SEQ ID NO: 2 - DNA, 27001 bp, H. sapiens
aaatgtcaccttcttaatgaagcttccttgatccccttcttgctaaagcggtatccccaccctcaccctacccagctctgttttccctcacagcaattaccaccatctgatctaccctataatttgcttatttactacattcgttgtctaggatatcagcttcacgggggcaggaatttttgtctgccatccatgttcatgtctgccgtgtccccagctagctagaacagagtctagcacaggagaagggatcagcatgagatgagatggggctggatctccaagggctttgactaccagactgaggagctgaactgtgttctgaggacatgggcaaagctatggaaggagagaaagatgcttccagatgccaggtggaaggtggattagagaggggcaagaaagaaggccctgggcccaaagagagcagggcagggacatgagggaaggcaggaggtgtccctgacaaggcccatgatggttacctggggactggaggagcaatgggctgccctgacatggggtcatggaggaggataacacagagaggaaattcagcagaggtctgattagaagggccttgaatgttgaagaggttggactttatactgagggcactggggagctatggaaggatccttagcaggggagtaacatggatttggaaagatcactttggctgctgtgtggggatagataagacggtgggagcctagaaaggaggctgggttggaaactctgggacagaaacccagagaggaaaagactgggcctggggtctccagtgagtatcagggagtggggaatcagcaggagtctggtccccacccatccctcctttcccctctctctcctttcctgcaggctggccccggctccatttccaggtgtggtcccaggacagctttggccgctgccagcttgcaggctatggattttgccatgtgcccagtagcccgggcacccaccagctggcctgccccacgtggcggcccctgggcagttggcgagaacagttggcacgggctttcgtgggtggtgggccgcagctgctgcatggggacaccatctacagtggggccgaccgctatcgcctgcacacagctgctggtggcaccgtgcacctggagatcggcctgctgctccgcaacttcgaccgctacggcgtggagtgctgagggactctgcctccaacgtcaccaccatccacaccccggacacccagtgatgggggaggatggcacagtggtcaagagcacagactctagagactgtcagagctgaccccagctaaggcatggcaccgcttctgtcctttctaggacctcggggtccctctgggcccagtttccctatctgtaaattggggacagtaaatgtatggggtcgcagggtgttgagtgacaggaggctgcttagccacatgggaggtgctcagtaaaggagagcaattcttacaggtgtctgcctcctgacccttccatccttcaggtgtcctgttgccccctcctcccactgacaccctccggaggcccccatgttgacagaccctcttctcctaccttgtttcccagcctgactctccttccgttctgggtccccctcctctggtcggctcccctgtgtctcatcccccggattaagccttctccgcctggtcctctttctctggtgacccacaccgcccgcaaagccacagcgcatctggatcacccgctttggtggcgcttggccgccaggaggcagcaccctgtttgcggggcggagccggggtgcccgccccctttcccccagggctgaagggacccccctcggagcccgcccacgcgagatgaggacggtggcccagcccccccatgccctccccctgggggccgcccccgctcccgccccgtgcgcttcctgggtggggccgggggcggcttcaaaaccccctgccgacccagccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacggtgagctcggaggggcaggggagccgggaggggggcccccagggggcgccggagtgccggggccacgggtaggaagtgactggcagaagaaactggctggaggaagaggacaccccggggcaaagggaacgtgtgatggtgggaggggggtgtccgaaagaggatggcactgagccccctaccacccaggtgtctggtcttggagaggaggagatagcgaagtggaccgcttctagagtgcgacagaaacatgcggggtcgtgggggcagtcccctagagggagacaagcaatagggggagggtagaaggctccctcttccaggacgcgttgaatgggggggggggtcgtggggtgccaggtgcagagaagggagcctggtgtgggagaagcgaagaccccagcatttgggaaaggagaggcgctggagaaagttgacccagagcttgggggtcctgaggtggaaagattcaagaaggacagaaaagctagatgaaggcaaccccagagggtgccaggaaagtgagagcggacccacttccagaggctgccaggaacacgcgggatgcgggggtgggggagagtcgtagaaagagaaacagaggtgcgtgtgataaatgtggggagaaagggacgggaggttattggaaggaggaggcaagcgggagaggagcggagactcgggagggcgcccgggatgcagaggtgtcctcggtgtttcacacagggacgtgagggacagagtggggagcccagcggaggaatcgagcttccagaagacctagagtcctgggtcatgggaagggctttaccgagaggggagacaggcgtgggaaaggtggtgtgagcggggaggaggagagatacccagcgccatccacgctgcattccccgcaggatgcaggggaatgggctgagcggagtccagccgcaggggaagtgctgggtggggggtgactctacaagaccgaggtgagaaaaccaagctgggaggagtgagaaagccccacgtgggtgccacgcgcggggggaggagcctgcgcttccaccaaggggcaggaggaccccgctgcgctaaacgctggcagtctggaccccaaagtcccagttcctctcagggtggctggggaaaccccagcgtccggcggcctcatccccctcccttccctttccttcccatgcccccggcggaggcggggatcgctcgcggagcccggggcgagacggggcaggtctggtccccgccctcctggctgcggcgcctcccgccctcctcaccccagctacgggcggggctcccactcagccggccccgtcggcatccatgcgaggacccaggcgtcccgcgtggttcagagccttggggaagatccctcaggtttcactgactcttgggcggtgtgggcttgcgggttccctgcccatttctgcgccagtttacagctccagcccaatgacgcgcactccgcacccaagtctcagccttaccttatcttccgtggcttcaccttcgttgtagaggttcctttaacactgacacctccaatcctcttctccccaacaaatgcacatgtgtctcgtctcgcacgtgtctccccatctgcctctttcttttcgtctccgttagtctttctgtccacgcatgggtctcctggtttttgtctctccgactattttctctccctttctatttttctcctccacggtcctgttgcctcgtctccgtctctgacatctccccgcctctccctctctgcatcaccctccaacccttcctctccctcctctctgtcttcctccctatctgtcttccccagccaaggctctgcctttcctttggggtttgctgagtaacctccgggccaagaatagggcttactggggctgggtggggagggagactggggaggaggaggaggatcgaagggggccgtaggggaggggtttcctctgcctttctcaccagtctcttttcacacccccactatgggaggctggaagcagttgccccagttgatccagcagttcatggcctgttccctcatccccatccccaaacttttcctaaactagaaaataccttggctgggcgcggtggctcacgcctgtaatcccagcactttgggaggccaaggtgggtggaccacctgaggtcaggagttggagaccagcctggccaacatggtgaaacccggtctctactaaaaatacaaaaattagctgggcggggtggcgggcggcgcctgtaatcccagctacttgggaggctgaggcaggagaatcgcttgaacctgggaggcagaggttgctatgagccaagatcgcgccactgcactccagcctgggcgacagaaactccatctcaaaaaaaaaaaaaaaaaaagaaaagaaaacaccttaattccttttctccccactacagccattcctacccagaatgaacttcactttccctaaccagctggggaagggagttaagggtggaaaactaataataaaaataagtagaagaatcacttaggggttaagagcacagtctctagctgggtgggtgtggtggctcatgcctgtgatctcaacatattgggaggctgaggcgtgaggatcccttgagcccaggagttccagcctgccgtgagccatggttgtgctactgcacttccgtctgggggacagagtgagaccctgtctcttgggggaagaaaaaaaaaaagcacagtctctggaatgagacagcttgagtttgaatcccagtctgcagctatgtgaccttggggcaaattacttaatctcggtgcctcagtttctcatctattaaacgggtataataatagtataaggttgtgaggtttaaatgtcttcatctttgtaaagtgctttagatagcatctggtaaatagcgctgttttgtggttttttgttgttgttgtttttgagacagagtcttactctgtcacccaggctggagtgcagtggcgcaatctcggcacactataacctctgcctctcaggttcaagcgattctcatgcctcaacctcccaagtagctgggattagagccgtccggcaccatgcctggccaatttttgtatttttagtagaaacggggtttcaccatgttggccaggctggtctcgaactcccgacctcaagtaatcaatccacctcggcctcccaaagtgctgggattacaggcgtgagccactgcgcccagccagcactcttagacgtatacatgattgagtgaccagtgtatgcagaattggtgccacatggcttgacctgggtcatctaatttcccactataatactggaatgggttctagagtcactctgtcttgtcttacacctggctcagagagaggaattcacttgcccaaggttacacagcgcttaaattaggagagtctgtgtgggaatgcagggagcttaatgttattgctgtttttcaaagaataacaggaaactgacattcagaaagagggtgttaactgcccatggtcacacagccaagaggcagcagaggcactatctgagtccaaagcctgtgcctttgatccttactctgcattttaggggctcttgcatttggggcaaggaaggccagagcaaacagagtggttccccttggtgccagccttagtcgacttgagagatgatgctacatgagagaggaatgtggttaggacctattttacagaggaggaaacaaacctagggggagaaaccacttcctcaaggtcacacagctaaaaagtatcacagtcagggctgggcgtggtggctcacacctgtaatcccagcactttgggaggccgaggcgggtggatcacgaggtcaggagatcgagaccatcctggccaacatggtgaaaccccatctctactaaaaatacaaaaattagctgggcatggtggcatgagcctttagtcccagctgctcgggaggctgaggcaggagaattgcttgaaccctggaggcggaggttgcagtgagcagagattgcgccactgcactccaagcctgggtgacagagcgagtctccgtctcaaaaaaaaaaaaaaattcatttaaataaataaataaataaataaataaataaataaataaataaaaggtatcagagactgactccaccccagagctgtcagctccaaaactcctagagttgggagcaccagctcccctcacctctgccaaacccctgatcgcctcccttcatttctccctgctagaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccaggtgaggacatgagccagaaggaaggtcagggcatgggctggagagggtgagctgtgaccaagggggtggctgtgggtcggctggttacaaggtccacctagatggtccctgaaggatagaagaacacaaaccatacaatcctagaacgctttttttttttttttgagacagtgtctcactctgttgccaagctggagtgcagtggtatgacctcagctcactgcaatctccgcctcctgggttccagtgattctcctgcctcagcctcccaagtagctgggattacaggtgtgcgccaccacgcccagctaatttttgtatttttggtagagatggggttttaccatgttggccaggatggtctcgatctcctgacctcgtgatccacctgcctcggtctcccaaagtgctcagattacaggcgtgagccactgcgcctggcctcttttttttttttttttttttttgagatggagtcttgctctgtcgcccaggctggagtacaatggggtgatctcggctcactgcaacctccgcctcctgggttcaagtgattctcctgcctcagccacccgagtagctgggattacaggcatgtgccaacacgctgggctaatttttgtatttttagtagagacagagttttatcatgttggccaggcttgtctcaaactcctgccctcaagtgattctcctacctgagcctcccagagtgctgggattacaggtgtgagccactgcacccggaacctagagcacttttaaatgttcagactctttgcatcctaggatgttaaacacttagaaggctggaatcttaggagttggactctttaaggacacaggattcttgaaagttggaatctctgaaaaggttgggggctctagaatcattctgtccaatatgacagccactagtcacatttagcttgattaaaatttaaattgtttaaaattaaattaaaaatttagggccaggcatagtggctcacacctctaatcccagaactttgggaagccgaggcaggcagattgcttgagcccaggagtccgagaccagtctgggcaacatgttgaaacttcgtctctacaaaaaatactaaaattagcctgttatggtgatgcgtgcctctagtcccagctactcagtagtctgaggtgggaggattgattgagcccaggaggtcgaggctgtagtgaactgtgattgcaccactgcattccagcctgggtgatggagcgagaccgtctcaaaaatatatatatataggccaggcgtggtggctcatgcctgcaatcccagcactttgggaggccagggtgggtggatcacttgaggtcaggagttcaagaccagcctggccaacatgacgaaactccatctctactaaaaatacaaaaattagccaggtgtggtggcaggcacctgtaatcccagctacttgggaggctgaggtgggagaatcgcttgaccccaggaggtggagactgcagtgagcggagatcataccactgcactccagcctgggcaacagagcgagactctgtctcaaaaaaataaaaatgaaaaagaaatcctcattctcactggccatatttctagtactctatagtcacatgtggttagcagctactattttggatgttgcagataaagcacatttccagaaagttctttaggacagcactgctctagaagatagggagcttccaagaggactggggcatctggaagggctggaggctctagcagtttctatgagctagaatccatatcagagggaatgttaactcataggatggtagatttcagactttcacagtgagagaactttgtcctatgttagcttggcttcttggagtctggggaattcagctttattctgcagtccctggagtggactatccagccccagaaaattcttcttttttttgtgtgtgtgtgtgatggagttttgctcctgttgcccaggctggagtgcaatggcaccatcttggctcaccacaacctttgcttcctgggttcaagcgattctcctgcctcagccacccgagcagctgggattacaggcatgcaccaccacacctagctaattttgtatttttagtagagacggtgtttctccatattggtcaggctggtctcgaacttctgacctctggtgatccaccctcctcggcctcccaaagtgctgggattacaagcgtgagcgactgtgcctggcccagaaaattattctacacagatgttgggtacctgcagcatctaggtgcggcatggcagactcttgggtttatagaacattagtcttctaaagcttctcaggctctagaagtggaaatcttgggatattatcaaacatttgaatcacaaaatgactttttttttttttgagacagagtctcactctgtcaccctggctagagtgcagtggtgcaatctcggctcactgcaacctctgtgtcttgggttcaagtgattctcctgcctcagcctcctgagtagctgggattacaggcgtgtgccaccacgcctggctaatttttgtatttttagtagagacgaggtttcactttgttggccaggctggtcttgaactcctgacctcaggagatccacccgccttggcctcccaaagtgctgggataacaggcgtgagccaccgtgcccagccggaatcattagaaatgacttctaagttactgagaattcagggtgtcaaatttgcagaaccgtatgctcagtaaaccccagaatgtttgcagcagaattttattttatttattattattattttgagacgaagtctcgtcctgtcgcccaggctggagtgcagtggcgtgatcttggctcactgcagtctgtgtctcccgggctcaagcaattctcctgcctcagcctcccaaatagtggcgactacaggcgtgtgccaccatgcctggctaatttttgtatttttagtagagatggggtttcactatgttggccaggctggtcttgaactcctgacctcaagtgatccacccaccttgggctcccaaagtgctgggattacaggcgtgagccactgcgcccggcccagaatgttagcagcagaattttagcattgtgggctgtccacgctgagtggggcttagcatttcaccaatgaggaaacaggcctcgagaaggcaagaaaacaccttcggctgagctgtgtgaaggtgacttgaccgcagcctgagctttttctccacccctcctctcatgggtactgttggggaggatgggtgccacaggaccacacaggtggctgtctgagagggtagtgcctgggaactttctggaagcctgtttggggaagcagatggggtgaaggattcagttagtgtatgtggggtcgtgacaccatctacccactgtctctctcctgccttcatcatcctctagaaatacagcaacaattcctggcgatacctcagcaaccggctgctggcacccagcgactcgccagagtggttatcttttgatgtcaccggagttgtgcggcagtggttgagccgtggaggtgaggattacttgtgtgtcccacccctgtttctccctggggtccaccccattgtttgtcctggggtcactttgcctaggacccctctccgccccatactggtgtgaaagtgctgagacctggctcccctctgtggcatggaaacctgagtgattggctaatctgtttcctgagcacctctctgccctgccctgtgatgggtgatgccaaggacacagcaatgaccaaaacagccccatccttgccctcacagagctcatatagtccattggctaggtagctggggaacagacctgtaaggcagtgatgacccagagtgggtgggactgggatggggagcccaaaggaggtacctgacccagcccagaatcagggagggcttcctggaggaggggcaccatagctgatgttgaagggatgagtagaatggtcaggaaaaggagaggtgtaagagtattctggggctgggcatggtggcttatgcctataatcccagcacttttgaaggccaaggaggatggatcactggagctcaggagttttgagagcagcctgggcaacatggcgaaacccggtctctacacaaaaatacaaaaattagccatgcatgctgttacgtgcctgtagttccagctgcttgggagactgaggtgggagaatcacctgagcctgggaggtcgaggctgcagtgagtcataatcacactactgcaatccagcctacatgaaagagtgagatcccatctcaaaaaaaaaaaaaaaaaaaagattccaggcaaagggaacagcctgtgcaaaggcctggaggccagggagagcctaactaactcatttgatctgtccaggagatgattgtctctgtctggtttctggcagggcccatggtgcccctatctctctccctcacctccctctcatttatgcttttattcaataattgcccacaggttcctgctgtgtgcctggccatgtgctgggtggtgtgggggacatagtgataaccaagacagccctcagttctgccctcatggggcccacagtcacttctggacacctaagagatcttgtcaaggccccaccaggcaggaatgacctagaatgatcaagaatgggatggggaggcaccagcagggaggacaaggctgggacagggaggcatgcaggatgggggaacgccctaagtgctgttgggagcccagagaaggcacctggacccatctggggagtctaggagggcttgctgggtgagggtgattgtgtggagactcacaggataaatagtagtttggccacatgaaaaaaaagggattagggggcctggcatggtggctcacgcctgtaatcccagcactttgagaggccgaggtgggtggatcacctgagatcgggagttcgagaccagcctgaccaacatggagaaaccccgtctctactaaaaatacataattagctcggcgtggtgatgcatgcctgtaatcccagctactcgggaggctgaggcaggagaattgcttgaacccaggaggtggaggtttcagtgagccaagatcgtgccattgcactccagcctgggcaacaacagtgaaactccgtctcaaaaaaaaaaaagaaaagaaaaagaaaaaatagggattagggagcaggtgggcactgggaggagtgtcctgggctgttcgacaaaggcctagaaggagctcatctgtcttggtcaatgctgagtcccaagcacacagcacaggcttcggcacattgtaggagcctagaaaacatcattcactcattcattcaagagattgatgctggctgggtgcagtggctcatgcctgtaatcccagcacttcgggaggccagggcaggaggattgcttgagcccagcctgggcaacatagtgagacctctgtctctacagagaatttaaaaagtagccgtgtaggccggatgcagtggctcaagcctgtaatcccagcactttgggatgccaaggcaggcggatcacctgaggtcaggagtttgagaccagcctggccagcatggtgaaaccccatctctactaaaaatacaaaaaattagccgggtgtggtggcgcatgcctgttatcccagctacttgggaggctgaggcaggagaatcgtttgaacctgggaggtggaggttgcagtgagctgatatcacgccactgaactccagcctgggcaacagagccagactccatctcaaaaaaaaaaaaaaaattagccatgcgtggtagcacgcgcctgtggtcccagctactcgggaggctgagatgggaggattgcttgggccttggagattgaggctgcgagaccttgtctcaaaaaaaaaaaaaaaaaaaaaaagagagagctgcagtgagagggcagagtggggtgggtgggggaggcaggaaggaagcccctggggtttgctccttccttcctcttcctccaggggctgggtgagctgcactctcagactggcttccctctcgccactcctacaggggaaattgagggctttcgccttagcgcccactgctcctgtgacagcagggataacacactgcaagtggacatcaacggtgaggcctgcttccccggccatgcccagttgtgacgtgtgtgcgtgtgtgtgttcccatctgccccacgccccacttatctatccctctgagagtgtgtgtgtatgtcccctatcccctgactcccacaccaaagcagggttcactaccggccgccgaggtgacctggccaccattcatggcatgaaccggcctttcctgcttctcatggccaccccgctggagagggcccagcatctgcaaagctcccggcaccgccgagccctggacaccaactattgcttcaggtgagccttgtagcctggatggaggccttccaggctgggggcatgactgccatctgctgaccaggtgctccaggttggacacctggctgctgctgtgcttgggctgagcatctgtctcctttagcttggagggctcaggggatgaggtctgggtgtaagaaccaggagtcttgtgccaggtgcggtggctcacgcctgtaatcccaacactttgggaggctgaggcaggtggatcacctgaagtgaggagtttgagacctgtctggccaacatggtgaaacccgtctctactaaaaatccaaaaattagccaggcgtggtgggcgcctataatcccagccacaaaaaacaaaaaaagaactgggagtcttgtaatgttgggatcaggttcctttttttttttttttttttttttttgagacagagtctcgctctgtcacctgggctggagtgcagtggcgctatcttgattcactgcaagctccacctcccgggttcacacgattctcctgtctcagcctcccgagtagctaggactacaggcacgtgccaccacgcccagctaatttttttgtatttttagtagagatggggtttcactgtgttagccaggatggtctccatctcctgacctcgtgatacacccacctcagcctcccaaagtgctgggattacaggcgtgagccaacgcgcccggcctaattttttgtatctttagtagagactgggtttcaccatgttagccaggatggtcttgatctcctgacctcgtgatccgcctgcctcagcctcccgaagtgctgggattacaggtgtgagccaccacgcccagccttgctttcttgctggagtgttggaatcacagaatgttagaaagttgaaccctgtgattgttggaattcaaagttggaaggtcaccatcttgaatccctaaattgttggcaggttgaggttctagaatgctgggattctagaatgctagaatttggtgttaattactgaaatggaaaatgtcggactcaaggaaagctgaaacattggaatcctagaatttggggattcaaaatgggaagatggcccatgtggaattcctgcaccactggccaattgtggataccagtgttggcagattggagtccttgaatattgggctcacagtggtagaattctgttttgaaatattcacatccacaaatgttggagttggagaaagcagaatattggtatctcagcatgttggaactcagtgttggaatgttagaatattagaatgctgggattttattaatagaaagtggcagcaggctgggcgcggtggctcacgcctataattccagcactttgggaggctgaggtgggcggatccctgaggtctggagttcaagaccagcctggccaacatggtgaaaccccatctctattaaaaatacaaaaattagccaggtgtggtggctggcacctgtaatcccagctactcggctgggattacggctatccaggtacccaggctggggcaggagaatcactggaacctcgggggtggaggttgcagtgaaccgagatcgcgccactgcactccggcctgggcaatgatgcgagactccatctaaaaaaaaaaaaaaaaaaaaaaaagaaagaaagtgtcagtttgaacctcttgagtgatggctcagaactttggaatgttgggatgcaatactggcatactggaattcaggaatgccaagatcctcaaaggttggaaagggggaatcctagagagttgaagatgctgtcagaatgttagaatcataagatgctggaatgctaatactgcaatctaagaaagctgaaatgttggattcctagaaagttgaaatggagagttagaatgttgtaaatttagagtgatcatctgaccttattcctcccttgcttaaaatattttggtcctgtctctgggctttgttaccggctgtttccctgccagaaaatcagccctgctcccaggctccttcaccaccagatcttagcgccatcagcccagcaacgctgtcaatgaacaaattgaggtcttttttgagacagagtctcgctctgttgcccaggctggagtgcagtggcgtgatctcggctcactgcaacctccgcctcccaggttcaagtgattctcctgcctcagcctcctgagtagctgggattacaggcgtgtgccaccacacccggctaatttttggtttttttagtagacactgggtttcaccatgttggtcaggctggtctcgaactcctgacctcgtgatccacccgcctcggcctcccaaagtgctgggattacaggcatgaaccactgcgcccggccacaaattgaggtcttgagtgccaacagctcctgacattacaaagggagatagccagccatgtcagcctccgtagggagggacccaatgttgcctaggaagtcttggcaaaaaatcaaacctgaaggccaggtgcggtggctcatgcctgtaatcccagcactttgggaggccaaggcaggcagatcgcttgagcttaggagttcaagaccagcttgtgcaacatgttaaaaccctgcctctgtacaagaaatacaaaaaaactagctggctgggtggtgtgtgcctgtagtcccagctacttgggaggctgaggcaggagtgcttgagcctaggaggttgtggctgcagtcagccaggatcacgccactgcatttcaggacagagcaggaccttgtctcaggaaaaaaaaaaaaaaatcaaacctgaatctgatcaggactgtagatcctaacagttttctagaaagacaggaacagaggaaatggtcagcaaaactcagactgcaagaaacctcacccagtttcttcatcaaatacaatgcaaagaaggggaaagcacacacacacacacacgtaccccccatgcaaccaaaaattcacatacaagttttgactccccaaaaacttaactattaatagcctactgtggactggaattctgctttatccacaacataaacagttgattaatacttttttttttttttggagacagagtctctgtcattcaggctggagtgcagtggtgcggtctcggctcactgcagcttctgcctcccgggttcaagtgattctcttgcttcagcctcccgagtagctgggaccacaggtgcccaccgccacgcccagctaattttttgtattttagtagagatggggtttcaccgtattgcccaggctggtcttgaactcctgaggtcaggcaatccgcccacctcagcctcccaaagtgctaggattacaggcatgagccactgctcccggccgaataatacatattttgcatgttttatgtattatgtcctatatcccctttttttttttttgagacggagtttcgctgttgtcactcaggctggagtgtaatggcacaatctcagctcacggcaacctccacctcccgggggttcaagcaattctcctgcttcagcctcctgagtagttgggattataggcacctgccaccatgcccggctaacttttgtatttttagtagaggtggggtttcaccatgttggccaggctggtcttgaactcctgacctcaagtgatctacccgcctcagcctcccaaagtgctgggattgcaggggtgagccacgcacccggccgtattctttgtttttttttttgagacagagtctagctgtgtcacccaggctggagtgcagtgaagtgatctcagctcactgcaacctccgcctcccaggttcaagcaattctcctgcctcagtctcccaaatagctgtgatttcaggcatccgccatcgcacccgggtaatttttgtatttttagtagagacggggtttcaccatgttggccaggctggtcttgaactcctgacctcaggtgatccatcagcctcggcctcccaaagtgctggccttacaggcttgaaccactgctcccggcctatgcctgcattttaaaaataagatagagaaaagaaaatgtccttaagaaaatcataaggaagaggaagtatattttccattcattgagtggaagtggcttatcataaaggtcttcatgttgagtaggctgaggaggaggaagaaaaggtgttggtcttgctgtctcagggcagaggtggaagaaaaattcacctgtaagtggtgaattttgtaagaggacccatgtagttcaaacccatgtccaagggtcagtctatatatagttgaccctgaatatatatacagagagagagagagatgattttgtagattttgaaaaaagtcctaagagaggccaggtatggtagctcacacccgtaatcccagcactttgggaggctgaggcaggtggatcacttgaggtcaggagttggagaccagcctggccaacatggtgaaaccctgtctgtactaaaaatacaaaaatcaggccagctgtcgtggctcacacctgtaatcccagcactttgggaggccaaggtgggtggaccacctgaggtcaggagttcgagaccagtctggccaacgtggtgaaacctcctctctactaaaaattcaaaaatgagccaggcgtggtggcgggccctgtaatcccagctactcttgaggctgaggcaggagaactgcttgaacccaggaagtggaggttgcagtgagccgaggtcgcgccattgcactccagcctgggcaacaagagtgagactcccgtctcaaaaaaataaataaataattaaaaatacaaaaattagcagggtatggtggcaggcgcctgtagtcttagctacttgggagactgaggcaggagaatcacttgaacctgggaggcacaggttgcagtgacccgagattgcaccacttcactgcatcctgggtgacagagtgagactccatctcaaaaattaaaaaaaaaaaaaaatcctaagagacatatcaaccaattgcaatgtgtagaccttacttgaatccttatttacacaggcttaaaaaaaaaagatatttatgagacagttgggtaaatgtgaactctgtctagatattcctctagatatcaaggaattgttaattttcttacatggtattgtgactgtgttttaaagtagtcttaattagagatacatttgaaatatttatggatgaagcaacacggtggattgcatgtgctttgagataaaccattgtcggctggctgcagtggctcacagctataatcccaccactttgggaggccaaggcaggaggattgcttgagcctaagagttggagaccagcctgggcaatatggtgaaaccccatctcccaaaaaaaaaaatatgaaaagtagctgggcctggtggtgtgtaccttttgtcccagctactcaggaggctgaggtgggaggttggcttgagccccaggagctggaggccacagtgagctgtgattgcaccactgcactccagcctgggtgacagagtgagatcctgtctcaaaaaaaaaaaaaaaagagatagtggataggggtagagatgcaatggaaattggccatgagttggtcattgttgaagctgcttttgtgtacatctgaaactttccagctggacgcggtggctcacacctgtaatcccagcactttggaaggctgaggcgggtggatcacatgaggtcaggagttcgagaccagcctgtccaacatggtgaaaccctgtctctactaaaaatacagaaaaattagctgggtatggtggtgtgtgcctgtaatcccagctacttgggaggctgagccaggagaattgcttgaacttgggacatggaggttgcagtgagccaagattgcaccactgcactccagcctggggcaacagagcaagactccatctcaaaaaaaaaaaaaaaaaactttcataggaaaatttgttcttaaggcagctcgaacactacctccttcctaacccctttctccaacatccaagatggatgtgaggcctctgggctcccctggggccctaagtagatctttgactcaacaaacagtctctgttgggtcttttatttataaaaagttttgtagcgacagggtctcactatgctccccaggctggtcttaaactccttgggctcaagcgattcaccggtcttggcttcccaaagtgctgagattacaggcgtgagtcactgcacctggcctaggtcttgagagactggacggaagaggcttggcaagggcaagggctgtgggtatcccaaggcccggtgcagggtaggccccgaatgtgtgctgaaaaaatgagtcagaggatcacctagacggcttgttcaacacggactgttgttcccagtcctgcacacaatttctgatttcagtggcctggggtgccacctgagaatctgcatttcccatagttcccaggtgatgctgccgctggtcctgggagctcacgtggagagctgctgacttggataacagtatcacagattgttcaaatcctaggatctcagaaccccagctagggttgaactgcatattaaagatgattctgagccgggtgcggtggctcatgcctgtaatcccagcactttgggaggccgaggtgggtgggtcacctgaggtcaggagtttgagaccagcctgaccaacatggtgaaacccagcctctactaaaaatacaaaaaattagccaggcatggtggcacatgcctgtaatcccaggtacttgagaggctgaggcaggagaattgcttgaacccgggagacggaggttgcagtgagctgagatcacaccatcgcactccagcctgggcaacaggagggaaactccatctcaaaaaaaaaaaaagattccggacactagttcttccccttttgtgagttcagaacttcctgtgagaaagtgaaagccaggactctttttcccagaaatacatgcatacatacacacatgtgcatatgatctcaggcactcatggccctccctaagcctgcccatgatggccccttggccctcagcttaacaccttattttatctagtagctcctacctctgaacagatgaggaaactgaggtccaaccaaattgggtggtacgggctgagttcacagggatctcagtttcttgacttccttctcaggcttctgtggctcttggaaaggctttatcttgcctctaggttatccggtgccacttgaagttgggttcaagcgattctcctgcctcagactccccagtagctgggattacaggtgcccgccaccaggtccagctaatttttgaatttttagtaaagacagggtcctcaagtctgcctgccctagcctcccaaagtgccgggatgacaggcgtgagccaccgtgcctggctggccccaccaatctatgagcagaggttgcagtgagccaagattgcaccactgcactccagcctgggtgacggagcaagactctgtctcaaaaaaaaaaaaaaaaaaaaaaggcattgtcaactttttcttttcttttctttcttttttcttttcttttcaagacagagtctcaccctgttgccctgggtggagtgcagtggcacgatcttggctcactgcaacctctgcttcttgggttcaagcagattcttgtgcctcagcctcctgagtagctgggattacagacaagcaccgccacacccagctaatttttgtatttttagtagagacggggtttcgccatgttggtcaggctggtctcaatctcctgatctcaggtgatccacccgccttggcctcccaaagtgctgggattacaggcgtgagccactgcacccggcctctttgacagttctaagaagtaaaataaaccagaagaaggcaggcagagagtaatagatattgggaactgttatttttcctattctgatttaaattcctttattatggaaaatttcaaatgtatacaaaagcaatgaagataatgaatgcccgtaaactctcatccagcgtcatcggtaattaagattttgccccatttcaaatgcagacatttttatattttcttgcataactgcaataccgtattgcactaaatggaactaacagtcacttggatattttggaaaggatggctcagaaaggtatctctgaggaggtgatgttcagtcatgtaactgatatttactagtacctactgcattccagacactgctttaggagttaagggtccctgagtgaaggacatttgagctaaggtttaaatgaagtgaaggggccaggtgtggtggctcatgccagtaatcccaatgctttgggaggccgaggcgtgcggatcgtttgagcccaagttcgacaccagcctcagcaacatagtgagaccgtgtctctaaaaagaataacaaattaaaaagaataaataggccagatgcagtggctcaggtctgtaataccagcactttgggaggctgaggtgggcggatcacaaggtcaggagttcgagaccagcctggccaacatggtgaaaccctgtctctactaaaaagacaaaaaaattagccgggcatggtggcacacgcctgtaatcccagctactcggaaggctgaggcaggagaatcgcttgaacctgggaggcggaggttgcagtgagccgagatcatgccactgcactccagcctggcagacagagtgagactctgtctcaaaaaaaaaaaaaaaaaaaaatagaaaaaaaaagaagaaatgaagtgaaggaacaagctggagtgggtatctgtgggactagcaaggcaggcagagggaacagcagatgcaggagccccgaaataagactgtctgaggaacagacaggacgccagtgtggctggagtggagtaggcgtgagagagggagttgagatcagccagatttgatagcaccttgtggctcacggtgaggacttgggcatttgccgtgagatggagccaggttcagagcagaggagtgacaagacaatttatagcatgcccatggagggcagagatttctgtctcttttttaaaaaaattgaaaaaaaattttgtagagacagggtcttgcactttgttgtccaggctggcatgcagtggtgttatcatagttcactgcaacctcgaactcctcgactccagcgatcctcccacttcatcctcccaaagtgctgggattaaaggtgtgagccaccatgcctagcctatttctatctgtttcatttgtcctcagtccacagtagatcccagcccagcacacagtagtagttcaataaacatttgttgcacaaatagagcagatcagtttacatggagctgtgttattttgtatgttccagggtgtggcatgccatgatttatttagcccccccgtggatggtcatctggcttcttacaggcttgtcttaagcattgcgtgaaattaattattacattgctcttagcactggaggaagtgcttaatctgtgttagtgattatcatgactatttgtgttgttattaacacagtgggtgcaagggagacccagatggagatagggctgggggggcaacctagggtgacacacgcacctggggaggaggggcatgtggcttctatggtggtagcccctccctgcccctgacgcgtctctcctgcctgcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtacgtctggcccaccgggctacgagatgcgcttggggggagccaggacggaggaagaggagagagaaagagaagtaaagtcagagaggtgagttggcaggatggggagaaagagagggatggggtggggaggggaatgaataaagagatggggagagaggcaggaagctagagaggggctctgagcaggggccagagggagatgagctatgaagacccacagagtgaagtaacagagggatgggggtgaaggggagaagagagacagggagatggaaggaaaaacgcagaaatggagagacaaaatgagagagacagatacagacacagagttaggccaaggagagacaaagacagatacacaacaaggcaagaggcgaagatgaggagggacagagactgagaaagaaaatcaggcgggcgcggcggctcacgatggtaataccaacactttgggacgctgaagcaggaggatcgcttgagcccaggagttcgagagtagcccaggcagcagactgagatcccatctctaccaaaaaaaaaaaaaaaaaaaaaaaaaaagctaggagtggtggcgcttgcctgtggttggagctactccggaggctgaggcgggaggatggcttgcgctcaggaggttgaggctgcagcgagccatgatcgtgccactatactccagcctgggtggcagagcgagacccagtctcaaaacaaaaagaaaatcagacaggtggggagagacagaataagataggatgttagaagataagagagaccgaattggagatgggaagaggggatgcggggagagacgaagtgagagaggctggcgcggtagcgggtgggggatggggcagtggagggccgttttcctccctccacgagccctgagccctgaccccgcccgccgcccgcaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggccaggcggggtggctcacgcctgtaatcctagcacttttgggaggcagagatgggaggattacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcagtgagccctgactgtgccactgtatgccagcctgggtgacaaagcaagactccatcttttttttatgttttttttttgagacggattttcactcttgttgcccaggctggagtgcaatgtcgagatcttggctcaccacaacctctgcctcccgggtttaagcgattctcctgcctcagcctcccaagtagctgggtagctgggattacaggcatgcgccaccatgctcggctaattttgtatttttttttagtagagacgaagtttcaccatgttgttcaggctggtctcgaacttctgacctgaggtgatccgcccgcctcggcctcccaaaatgctgagattacaagcatgagccagcgcacccagccaagactgcatctttaaaaaaaaaaaaaaaaaaagactgggcacggtggctcacacctgcaatcccagcactttgggaggctgaggcagggggatcacttgaggtcaggagtttgagaccagcctggaccacatggtgaaaccctgtctctactaaaatacaaaaaaaggctgggcgcagtggctcacgcctgtaatcccagcactttgggaggccaaggcgggcagatcacgaggtcaggagtttgagaccagcctgaccaacatggtgaaacccagtctctactaaaaatacaaaaattagccatggtggcgcacgcctgtaatctcagctactcaggaggctgaggcaggagaatcgcttgaacctgggaggtggaggttgcagtgagccgagatcacgccacttcactccagcctgggagacagagtgagactccatctcaaataaataaataaaaataaaaaatacaaaaaaaaattagtctggcattgtggtgcacacctgtatcccagctactcaggggcctgaggcaggagaatcgcatgaacaggggaggcagaggttgcagtgagctaaagtcgcaccactgcactccagcctgggcaacagagtgagactccatctcaaaaaaaaaaaaaaaaagaacttgcactcaaggaagacaggagccatggcagggtttgacctaggcgctcagagggtccgccgtcttgctcatggttgggagtggacagaagtgtcagggtgagggtgagcacagacagaccagctgtccaggcaagaaatggcagcagccacaggtgggtctccctctgctgtctccatttcttcccatctgctatggtcctgcttgtgaattctctcctgcttcctctttctcctgcctctcagtttctgctctttctcctaggtttcacttccccacacccagtgattgtcctgggaggaaggacagtatgagtgctgcggttcctttgcctgtgggcatgagagctgttggcagcactgggtctgggtgccagggacctggggccctcccagctcagcataggggtagaaatgtggttactcctc

SEQ ID NO: 3 - DNA, 21001 bp, Mus musculus
gttggcctcgactccatctccaggtgtggtcccaggacagctttggccgctgccagcttgctggctatggcttttgccatgtgcctagcagcccaggcactcatcagctggactgccctacatggaggcccctgggcagttggagggagcagctagcacgggctttcgtgggtggtgggccacagctgctgcacgcagataccatctacagcggggctgaccgctaccgcctgcacacggccgcgggtggcacagtgcaccttggtatcggcctgctgctgcgccacttcgatcgctatggagtggagtgttgagggacttcattgctactaatcgccatcatccactgagcacataatggcacagcagtgaagcaggcacgtggactctggagactgtcagtcctgatcacccccaagtcatggcccctatgccatggactgcaaggccaggaactcccctgtggcccatgctccactgaactacggggtagaaagggctgtgggttggagggacatgagcagggcccactgtttggactgtcgggggcacttaataaaagcgtgtatttctcatgggtaaggtgcctccttgtatccgctaaagctctcagacgtcggtctccttatttcactctccttgacactctcatccgcaaagtctgcccctgcctttcgctctccaccgcctaggtccccacttctaatacaggactctgttttctcacgaagtcccaatttttcctttgacccttcaacaactcccaaatccaccggggcgctttggttggtcaccggctttagtagtgctccgccgccaggaggcagcaccctggaggtggggcgaggccggggtgcccgccccctccccgcagggctgaagggaccccccttggagcccgcccacgctaagatgaagacagtgcccccccatgccctccccctggggctgcccccgccccgcgcgcgcttcctgggtggggccggggcggcttcaaaacccccgccgccccagccggtcccgccgccgccgccgcccttcgcgccccaggccgtccccctcctcctcccgccgcggatcctccagacagccaggcccccggccggggcaggggggacgccccttcggggcacccccggctctgagccgcactcggagtcggcctccgctgggagccggcaaaggagcagccgaggagccgtccgaggccccagagtctgagaccagccgccgccgcagggaggagggggaggaggagtgggaggagggacgagctggttgagagaagaggaaaaaagttttgagacttttccgctgctactgcaagtcagagacgtggggacttcttggcactgcgctgtctcgcaaggaggcaggacctgaggactccagacagccctgctcaccgtcgtggacactcgatcgctacccggcgttcctcagacgcccctattccggaccagccctcgggagccacaaaccccgcctcccgcgaagacttcaccccaaagctggggcgcaccccttgcacgccgccctccccccagcctgcctcttgagtccctcgcatcccaggaccctctctcccccgagaggcagatctccctcggacctgctggcagtagctcccctatttaagaacacccacttttggatctcagagagcgctcatctcgatttttaccctggtggtatactgagacaccttggtgtcagagcctcaccgcgactcctgctgctttctccctcaacctcaaattattcaggactatcacctacctttccttgggagaccccaccccacaagccctgcaggggcggggcctccgcatcccacctttgccgagggttcccgctctccgaagtgccgtggggcgccgcctcccccatgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacggtgagctccgaggggccggggagccaggagggagcccccagggggcgccggagtgcaggggtcaccgggaggaaattaccctcagaggaaactggctggaggaaggggagccctgggggcaccgggacaactgtgtgggtgtcccaaagaggctggcactttcccgtaccccaggtgtctttgagaggaatagacagcctgccaaagtgatcctctcctagaaaagcaaggtcgtggggagagtcccctctagaagaggagcatttggcaggttttgttttaagttcccctaatggtgcctaaaaatgcagggctcgctgggtggtctctttccagaaaggtagaacccagctcaagagaatggaaggtgtccaacatttcggaatggagatgcaatgtggaaagccgaccccagaccgggtcgtgagatggagagaaaacctaggggatgggtaccctagaactgccaaataaaatctctagtgggcttactctcagaaagcgacaggcgcacgctagagagtgtgatctgtggagtcactacggggttacaagtgaaaaaggaaagcgacgttgttgggagaaggactctagagaggagcagagagggtgcggaaaatgagcccggaggtgtcacgagtgttttgcttgggggcgtaaaagagcgcgcgagccggcaggcggagcccagtgaaggaagcgagcgagtcctcaagaaatctagaatactgggtcattgggaaggagtcataaaaaggggagactggcttaggaaggtggccccccgcggcagccaggcagacggagcagggataccatgccatccaggagtgagtggcgagcgcccctctcctgggcgtggaagggagtggctgagcctctggagtcgagccctagaggagcagctagaagtaggggagaggcagtcttcaaaactcaaatgaaaaaaccgaatcggtaaggatgaggaagcccccacgtgggtgccacgcgcgtggggaggagcttgcgcttcttgggggggtaagaggaccccacggaactgaacgctgacaggctgtaaccccaaggtcccagttcctcctaccggaggctggggaaaacccagcgtccggtcgccacgccctcctcccttccctttccttcccgtgcccgggaggaggggagacagagatcgcgcgcggagcctggggcgagacgaggcaggtccggatcccgccctccctaggctgttagtctcccgccctcctcaccccagtagtgggcggggaaactcacggccccgccctgtaggcgcccacgcgggacccaagcgtcgtgtgtgaccggggaactttaggtgtcagtagcttctccaggtttcaccttcctaaccccagaggtggacttaaggattatttgcacattgctggacccaattgtggctcaggcctaatgacacccctccctagccacgacttaatgtgacctcttcgttggagaagtcctttactctcctgagactgtatttcttacgcatatatctcttccggcatgtgcctctcatttccgtctcctctcccacatacctattcatctctatgtcaccccatttccctctgcgacacccacaatccttcctgccctcctatctctcccctatcggctacccccagctgagatcccatccctccattgtggtttgctgagtaacaaccgaaccaagaatagaggccctctgtccttcatcgacgtctgggatcaccatctcatccccactacaagaagccagaagccccagcagacgcagaatttcgggattttgccccttcgttccccagtgcagacatcctttaggctaggaagccctttagtgaatggggctcccaaacctgcagtcactcttacctagaaatcctctttgctgctaacacgaccaggaaatgggctaaagaggggagtaactaatgacactgatgacaggcaaatgataataaacccccactggccaggaaatggcagagttttgggattaggaccactgtctggttgtctccatctgatccccccctcccctgcttatcctcattcccactcagttctccccctccatccacctcccatgacttactgactcgctgtgcaggggtggattggtacggggtgcgggcttccccttctcaggagaggagggggtgagggaaggggatgtgaggggggactggaaggagggaaagctgaagtcgggttataaagtgaatgaatgaatgaatgaatgaatgaacgaacgaacaaacaaataaatgggggggggcaacccaatcccaggctgccaagtctgtctatgactgtagtcccagtactggggagacaaaggcaaaaggactgcttcaagaccatccttggatacacagtgaatgtgagactagcctaagctatgaaatcctgtctccagaacaaaagaaaataaacaaaaaaattagtccctggggcgggagagatggctcagaggttaagagcactggctgctcttccagaggtcctgagttcaagtcccagcaaccacatggtggctcacaaccatctgaaataagatccgattccttcttctggtgtgtctgaaaacagcgacagtgtactcatatatataaaataaataaattaatttttaaaagcttttaaaaaattagtccctgtaataaggtggcttgaatcttaccatgttgtgaagtctcaatgcctttatcttttgtaaagtgtttaaaatgatatctttaccatgcactgtattcccctcacctttattagcacacacacatgtgcacacacacgcacgtgcgtgcacacacacacatatgtaggctaactcccagtgaacagcttcctgactcaggctcaggctcctcagctttggctgcagactcacactagctaaaatacactttattatgtttagcttttccccagtgtctcatcagctctgttaatcctggatttgattggtgtcttacagttaaagagagaagtttggccgctactggtggcacaggcctgtaggatttgctgaaggaggcagaggtgggaggatctggagttcaaggccagcctgggatacacatttttctgccagccagtggtggcgcacgcctttaatcccagaatttgggaggcagaggcaggcggatttctgaatttgaagccagtttggtctacagattgagctccaggacagccagggctacacagagaaatcctgtctcaaaaaaaaaagaaaagaaagaaagggagggagggagggagggagggagagaggggggagaggggaagggaaggaaggaagaaaagaaagaaagaaagaaagagagaaagagagagagaggaaggaagaaagaaaggaaggaagggagggagggagggagggaagaaggaaggaaggaaggaaggaaggaaggaaggaagggaggaagggaggaaggaaggaaggaagaagccacaggtaacatagtgtttcagttgcagccgcctaggctgggaatcagaacttaatggtacttatttcatccaaggttagaaggcagttggcccagtagtagagcacttccttagaatccacctccaaggggctgtgtgtagctcagcgaccaagcgagcgagcgctcccacctagaacctgctgtgggagtcactcagtgcacgagcacccaacctgagtagcagcagggaagctttctgatcccccaaattatgccgtgcaatttactctacaacttctgtttggttttgtttgaggcgtggtctcctgcagcccaggtgaggtcagccagttaagaacattagagaggggcctctaagagcagtcaagataaccagctgttcttccagcgggttggggtttccaggacctacatggcagctcacagccatctgtaactccggttctagaggatctgacaccctcttctggcttccacgggcactaggcatgcactctgcacacagacacacggatgcagccaaaacatccctacacataagtaaactgaatgttagaatgtgaagtggaatgccaacctgagttagagcccatgggctcagaagcagggtgtctctgtttcttccatccttgtcacagacctgacctgatgtcttcctccttctgctcctccccacccagccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccaggtaaggctttgagcctgagaggtggcacacaggtcagccctgctgaggtgtggccaggtggctcagtttgaaggctcctgggtacagagccaggcctgagggtagagcacagtgtcgaagcctaaggtttttatttttatttttgtcttgttttcaattggttggttggttggttgggtttttcgtgacagggtctctctgtgtagccctggctgtcctggaactcactctgtagatcaggccggcctcaagttcacagagattcgcttgcctttgcctctccagtgctgggattaaaggtgtgtgccaccatcacccagagaatcctaaggtttttttaaaaaccgctcagattgcatcctattagttgaatttgggagtttgaactgtaaaaatgcagaaagagccagggatggatatctggctatgtatgcatgactagaatcccccagtgaggggcagggggcgtggctcagtgggagagcccctgcctagaatcccccagtgaggggctgggggcgtggctcagtggtagagcccctgcctagaatcccccagtgaggggctgggggcgtggctcagtgatagactgcctagctaccactaaaaatgcattaggttctgtcctcagcacagaggtgaaaaaagtaacagtgaggaagaaaaagaagttggagtctttaggagcagacacttgcaaatcctagcacgtgggacgggggaggcaggagatcaggagtctgaggccagcctggactacatgagaccttgtctggaggctgaggcaataagatctcttaaacccaagcattcaagtccagcacaggcagactagtgagatcttgtctcagacagagaaattaagcatttcttgtagtctcttaaatcagcagtcaaatgcattttttttttttttttgacaagagttccctgtgtagttctggctgtcttggaactcagagctccactcacctctgtcttctgagtattgggattaaagatgtgtcagccaaaaatttttcaacccgggaggcagaggcaggaggatctcttgagttcaaggcaagccgagtctgcagagttagtgccagaacagctatggctacatagagagaccctgtctcaaaaaaataacaaaagtaacaaaaatttaaaaaaaaaaaattgggaagactgttctgtgtgtgtactcataataataattataagtaaaataacaaactataaataaataagaaaattactcccaaactcagggtaataaaactgctgaactgtgcacaatagtatgagcaatgaccacttgcattgtgagtcctccctgctgccatgttgcagcagcattattgagacccggcaaggccagcaagtgcccatagcttgcacagtacctcatgcacagctgaggagcctcatgggacctgaccacagcctgggttgtcctccttgtcccatgtcctattccagtctgtctccacccctcctcccctgcccctggctgctttctgttcttgggggctgtatgtgagagtgtggcctgcagggctcatatgtgtcagaagccagtgtctggggctggtctggaagccagtttgtgagacaggcagcatggggttacagccagattatctgggggcaagatggcagcgaccatactcctctttgctctctttgtttccatttagaaatatagcaacaattcctggcgttaccttggtaaccggctgctgacccccactgatacgcctgagtggctgtcttttgacgtcactggagttgtacggcagtggctgaaccaaggaggtgagattgcttgtctttgtgccatcctccagtggggccactcctccagtggggccactcctgcttgtcctggcgtcaacctgcctagcactgtgttccccaccccccacctcacccctgccacacacatgcactcgatagtgatgtcaagaagtagtactccagccaggcagtggtggcacacgcctttaatcccagcacttgggaggcagaggcaggcagatttctgagttcaaggccagcttggtctacagagtgagttccaggacaaccaggggtaaaccctgcctcgaaacaaacaaacaaacaaaacccaaaacaaaaaaagggtcaatctgtagagggatgcttgggataggtggtgacccaaacagtccctgtccttgccctcagtagacctcatagtcctttggtagttgaaggaggagttgacttctagctaggtgctttatgaatagtgaggaatggggttgagtactcaagaggtgcctgactactgtctggattcaaggagggcttcctggaggagagggcatctgaggtaatgtctctgagatgaataaaggcaagaaggaaaaggagatgcaggagcaccctgtatcaagtaccctgtatctgcagccagggagatcctgacatgtccatctgtctaggagtgaacaattttccttgtctcttatgggtcccacagtatctttccagatactggtaaggtcttgtcggagctctagcaggcagtaatggccgataacgatgaaggggagacctatacagagggggcaggatttagatgagggaagccaaaggtgctgtggaaacccaggcaaggttaggatcttggagggcttcctggatgagaatgtgaagagactcatgggaagtgtagtttagccacatggagggtgagtcttgagtgggctggcaccaggaggaatgtggaaggccactggacggaggtggagaagggttttgtctggtatacagtcggcgcttagacagtattattatgtgtccactcaaggaacatctcccttgagccagtgagatggctcagcaggtaaaggcacctgctgctgtgcttaacgacctgagttcaagcccaggacccatatggtagaaggagggagccattctgacctccttgcccaccttggcccatgtgcacccccgtaaataatgaggagaagatgccagcttaggttttcctggtgcctttggctatggcttcttctgtggccctggccaagccgctttctcttggggtgacttggagttaaaggagtttgaagatcaatggcgttaacacagtttagatcagtgacgggcatctgtgttagcgttgaacatcctgcgacaccaacactgctcgtggcccttgacacctgggtcacaatagatgtttattgttatgatcgaggcccaggctctacagagcacagagataacaagaacccagggcagcatgcacgatggtacacaaaccagcggagccctgcgcttgtgaggcagaggcaaggggaccacgagttcaaggtcattcttagctccatagtgagttgaaaatttgaggccagcttgggctacctgagaccttgtctcaaaagcaaaagagggctgtggcgagggctcaggggcaggagcatctgtctcgaaggcgcgagcgaggacctgagttcttgtccctggcactcaggtaacaagtcaggcattgtcatagtgtgtgcccgtaatcccagccttgttgggagttccacgtgtgtatggagacaggctgacacacggctctatgacctgctagcctggccaaaggactgggctcaagttcagagcccaggcaataagggagaaaggaatagaggaagacacttgttacccagctataagcctcctcatgtaccatacacagaacacatcacccatacatattctctgatatacataaaatatgcattatatatacacacataaatatatatatatacatatacatacacacacatatatatacatatatatgtacatatatacaattttaaaaattacaaaatgaaaggaaataaggcaggatactcccctgtgcatgttttccttttttgggtttatttgtttgtttgtttgtttttgtttttgttttgttttgttttgtttttgagacagggtttctctgtagccctggctgtcctggaactcactctgtagaccaggctggcctcaaactcagaaatccgcctgcctttgcctcccaagtgctgggattaaaggcgtgcgccaccaccgcccagctatgtttttctactgagaccaaagaagtaaatattcatgcagaggtgtcagagcccctgcttgccgtggagaaagggaacagatgccacatttggagaaggacgtgtctctttcagagcctttcccgaggggttgatgtttgagctgagccctgaataaggggagaagggtccagcaattcaaagaataggagcaagaatgaagtgtggggtgactagccgggagcccccaaggctggcagtttcctgtcccctcttccccctcatatcctcagactcccttcctctcccgcagacggaatacagggctttcgattcagcgctcactgctcttgtgacagcaaagataacaaactccacgtggaaatcaacggtgagctaacttcccgggtccatcctgacacaggcccctcatggtctgtgtgcacgtgcatgtgtgtgaaatctcacccatccctctaagtgtgtgtatgtccttcaccacaacatccaaacccaacagggatcagccccaaacgtcggggcgacctgggcaccatccatgacatgaaccggcccttcctgctcctcatggccacccccctggaaagggcccagcacctgcacagctcacggcaccggagagccctggataccaactattgcttcaggtaaacactgtctctctccgaggctgtgcaggctgcgggctgactgcagcctgtggactggtgtgccaccatgtatgcctgcagggcttggggacaagttcctggttctgtgggcagctgagagtcctagaatgaggggtgttttcttcctggagtgctgggatcagggaattcaacaagagttggaagtctgttggaaggttgtctggttgggttttaaaattcttgccaggcttctgccactttgggatgctaatgaaagaatttactgtgagccatggagcgtgttgaccccaggaaagctggagtgctgggatccaaacatttgggttccgaatcagaaggtggtctgcactgggttcccacactcttggcagatggtggtctccagtgctggtggatgagagcggcaaatcctgggtctcagaatggcagggctggcagggagatgttagcatctagagtggaggtggagaaagccagaaagtgagtttcctgagggccagcgctttatctggcagtggtgcaccctgaggaggtgagaagatagaacttctgagatgtatgtaggcttggaagtgtgaagtgttggaatatagtactgagatagggaactctgaggagacaagatctctcagagtcagagagtgggcgtcctagtgtatccagaatgcccgtaccaagggccaaagctttttggtcattgtttttaagttatatctctgtgtgtgtgtagggccattgtgtgtgtgtgtctgtgtgtatgtaggggcatccatgccacaaaactgtgtgacggtcagaggacaactgttgggagtccgttctccttccaccatgtgagtcccagatataagactcaggtcattgggcctggctgcgagcacctttgcctgctgagccatcttgttagttctcctttgtttggttttgttcgagacagggctttgctgtgtaatccaggctggcctcagattcactcctttagcccagactgtcttgaattctctgtcctgggattacagatgtcacacaaccacccagttccagaatggtagactcatattaagcctcggtactgtggatgagagaaagaaattggaacgttagatcctagaaggcatgaaattcaggggtgttgagagatgagagcagtgagctgacctatcccttacctgcttcagactttatagagttctgtgttacatatcggtccttggctggccggcctcctcctcctcctcctcctcctcctcctcctccttctcctcctcctccttgtcttcacctttggttgtcagccttcacataggatcagatgtccggtcttcatactgcaggttactaagagatgcaggaagctatgtaaactctgttcctcacatactgggaaacaaactcagggaatcatgaaaatctctttgtctgtctgtctgactgactgactgtgtctgtctgcctgtctctctcttatatttgttttgcctatgcctgatgtatatgtgggctctgggaatccgaaaccaggtcttcatgcttgcacagcgagcacttaattccctgagccaagtccccggctgtgacaagcaaaattttgcatggaagaacaccatgccatcgagcaaggcctatcagacacatcaaccatgatctgatgggaactgtagctccaactcccatgttgcagggaactgcaacagagggacagactaacaacattagccagcaccacagggacgcacccagcaaagtgcagatggtgggaaacctagtcatttcttcaacagatacagtgcaaagaagggaggggaggatatatagtccacactactagaagagagaggaagagagggctgagaggttggaggaggaggaaagaaaaggaaagggaaaaagatgtttatgggacacagtgggaaacatgaaccttgagtagtatttgattttagtgatttaatgttaaatttttgcctggggaagggggctggagagatggctcagtggttaaaagcactggctgcttttccagaggacttgcgttcagttcccaccacccataataaggatcacacctgtctataattctagctctagaggatttgatgccctcacacatgcataagtgcaggtaaaacaccaatatacattaaaaaaaaaaacaaaacaaaacaaaacaaaaaaaccaaccctccctaggattttgttgttgctgctgctgcatttgtgatactgagaaaacccagtaccttgtacatagtaaagaagcactctaccactgagccacgcccccagcccctcactgggggattctaggcaggggctctaccaccaagccacgcccccagcccctcactgggggattctaggcaggggctctaccaccaagccacgcccccagcccctcactgggggattctaggcaggggctctaccactgatttatattttatattgatttatacttttgattcatgtttaaattgtaaaatattcctcactttctaagagaaagtgaacttttctacaaaagttcattaaacaaatgaaaaaactacagtggcggaaagaaagtaggtggaatgaaattggccacgagtcagcaactgttgtcatttacagttgtggagatgcgaattagttaagaatccataattcagaagtccaaaatctcgctggccatggtgagcaaggctgtaatcctagcgctggagtaggaacgggaaccgcaggaggtcaaagtcattgataggtacgtcgtgtgtttgtggcaaccaatcagaccctgcctcgagaaagggggtggggcatctggaaggatgaggagcacagctgctctttcagagcacgcagattcatttcccagcaacctacatggctgctcccagcaatccatagtcccgttccagggaatctaacgcctcctcctggcccccatgggctctgcacgtatgtggtgtgtgtacacacatgttcagagaaatactcctacacataaaactttttttttttttttttccgagacagggtttctttgtatagccctggctgtcctggaactcactctgtagaccaggctggcctcgaactcagaaattcacctgcctctgccgaccgagtgctgggattaaaggcgtgtgccaccacgcccggctcgctctctctctctctctctctctctctccctttctttctttctttctttctttctttctttctctctttctttctttctttcaagttgacaggtgtggtgcatgtctctgatcccagcattcaggaagcagaggcaggcagatctctgagtttgaggccagtgacaaacaaatttcaggcaaatacatagatcctgtctcaagaaatatgaatagtaataaaaataaaataaatatgtaaagtctatgtaaatatccccttaccccaaaaagctatacagtctagaatgtttctggtccctaacgtttctgaaaaggggtattccgtttatattgtaacgcactgcttctgtgtctcttcacaattttccatggcaaagctgtttttatgccatcttaaaaaccacctcccccggggtcctcagggctcctcacagattaggccctgctgggtctcagactggcaagggaggcttgaaaaagcagtgactctgaaaaccctgagaccgcgtcccagtagggcgcccccatgtgccaggcggtggtggtgcacgtcttcaattccagcactcaggaggcagaggcaggtggatctctatgagtgcgagtccaaccaggcttaaaacgtcctgcctggactaggctggcttcaaacttgcagcgatcctcccatttcggcttcttgactacagttgtgcacagaggtgcatgcctgcctggttcacatgtgtcaagttttattttttattattttcttttacatgcatgttttgtctttgtggatgtatacgtggtgcatatgtggtgcccaccgaggacaaaagaggatgttggatcccctagatctggggttacaagttggttttgagcctccatgttggtgctaggaattgaatctgggtcctctgcaagagcatccagtgcacttgacctctaagccatttcccagttccttgaccacattttagagttatttttggtgtcatgaggaggaacctgcattttccttccttgaattactttgtcctgattctaaaaataaccccagccactgctaagaatgtggaaaatccagaaagtcatcacactggtgaacgtaaaacgccactgtcagcatttagtatacatcctgccagcctctttgtttgtaatggaatttgaattatactatacctgtatctttacatcatgattctgatacatgtcaggaacacagaggctcaagtgtccttgctggtcagcctgggctacaaactgaattctgggtcaacctaggctacgtggtgagaccttgtcttaacgctcctctcttttgaggggatcatggagggtgaggtggcaattgctggtaatcccagcacttgggaggctaaggcaggagttatcagaagttcaaggttagcctgggctacataacaaaacccagtcttaaagtaaaggactggccagagacaggagggtctggggagtttgagggcagcctgatcttcgtagtgaatactaggacagccagggctccataaagagattatctctagtagaaatgtttatcagttaattcatgtactcaacatttgctgaacatgtatacattaagagacactttcccatggagagatgaggacagataatatttagtacaaacaagctgaatcattccgagacggaaaataaaactggtgaaatggccaggttagaagccaggccaggaaaaatgaatattagagcttggtgtggtgcacgctcgtgatcccagccctggagaggcagaggcaggaggatcaggagttttgggttggccttggctacatgaaagcctgtctcaaaaaagacaaataaacacaagaaatcctggggatgaattaatttttctgttctggttaaaattgttttagggtagaaattccaaatgtagacagaaaaagcaaaggtatataaccctcttccagcttccagcattctcaggattttgatgactcacttcagacactctggagctggagacttggcccaacagttaaaagcactggctgtttgttcttccttaggacctgggttcagttcccagcacccatatggttacttagaaggacaaccatcactaactccagttcctagggacttcatgccctcttctggcctccttgggtactgcacaaacatggtgcacatcaaaacttacgtaggcacacatatgcacacaaataataaaatgaatacataaacctttaaaacctagagcaaggggctggagagatggcccagtggttaagagcaccgactgctcttctgaaggtcctgagttcaaatcccagcaaccacatggtggctcacaaccatccgtaatgagatctgatgccctcttatggtgcatctgaagacagctacagtgtacttagatataataacaaataaatctaaaaaaaaatccttaaaaaacaacaacaacaacaaaaaacctagagcaagccaggtggtagtgatcccacctttagtgccagcactcagaaggcagagagacaggtgtattcttaagttcgaggccagcctggtctacatacagagtgagttctgggacagcctgtctcaaggaagcaaacaaacaacaccaaaagctttatagaaattcagtttcgccgggcatgcctttaatcccagcactcgggaggcagaggcaggcgaatttctgagttcgaggccagcctggtctacaaagtgagctccaggaaaaccagggctatacagagaaaccctgtctcgaaaaaaccaaaccaaaccaaaccaaccaaacaacaacaacaaacaaacaaacaaacaaaaaaaccaaaaaaaaaaaaaaagggaaaagaaatacagtttcttgtagtaattgtagtactgaattcccagggtggaatttaacaattgtttggagggagagtttggaaggcaagtggaaagggctggtcagaggaggcactgttcatttatgtaactgatactggacacctgcttcaagcaagatggcatttttaggagttaaagctccctgggtggtagaaagtatctcacatagcccaggctagcctcaaactcactatgtctccaaggaggacctggaacttctaatcacccttgcctgtgcctcccaggtgttgggatcgaaagtgtgagccctcttgcctggctatgactgggtctgaggactagagaggaagcccatgtggctgtcagggtgggcgagggaggacactgaggaaagccagatagactcatggtgataacaggcatttgacgtaaacaggaggaaggagtgagtagctatggcctccaaaggcttccatctgtcctgtttgtgccattcaggagcaccggccctagccccatattcagtagaggtttggtaaatatgtgtcctacaaataggacgtgcaggcacatgttggactgtgaattactcctgtgtgttctgtggggatgtgacaggacttattacgtcctccccgagagtgctcatgggactgttgctagccctgcactggctctcaagtgcctgcccagtgggaggtgcttcactggcagcctggatggttgtagtgtcactattgtcatcacaggcgtcctggagagcctgagctggacaggaggctgagggcagccgagggctggagcgggctgcagggttgtccctgcttctgacctttgttcctcctgcctgcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtgggtctggctcatgggacaggctggagcagaggctggggtggtagggagagaatgggaagcttagagcagtggggagtcagcgggatagggtgacagagaaacggagaagagcagggggggggtgcagagcagagctggagagatggaaagaaacagattgggtgagaaaggcaagagagctagtgatgtggagagatacagaaaaggaggtggggagaattggagatgggagagtaagtgactgaggcagacaggaagaggagataggatgaagagagagagagagagaggaagagagagagagagagaggccgagatggggacagatgagggacagagaacaatagggagagacatatatagaaatagactgggagagatagggaaacagagatggaagttgaggaggctcagagataagagacagaaatgtagagatcctggtggggagagagatagggggagcccctgtactctggcagtgaccccggcccacccgcaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctgaagccccgccccgccccgcccctcccggcaggcccggccccgcccccgccccgcccgcagcgcccggggctgtatttaaggacacctgcaccccccctcaagcccacctggggggcccattaaaggtgacagaggactggtggtctccgtgtcattggccgccggattgcgtctccctcttagctgacgcccctaccacatcttgtccctttctgtgcaatcgcctccagcactccctaaggaacgccggctttgatcccagaattccagggggcgacgctgaggcccagggaaaaccctattagactcaggtatcatctctttaggcaccgttcacagtgctttatacctctgagcatccacgaggcaatccgggagagcctgaattcctgctgggatgaattcatttcttggatgatgagtatatggaagagcaaaaagtgctgatacagtgggttgtggtggccccatgtgacaagtccctgcattcaggagactgaggcaggcagatagatatagctcaggctagctcagatataaccaggctactggttcagagttgagtgtgtggtactgaaatgcctgcagtgggaaggcaagggttagataaggaactctggggcttccgggttacattagaccctatcccaaaaaacccccaattcacagaatgtggtggcacaggcctttaagaccagcactcaggaggctgtagcagattggtctctgagtttgaggccagcctggtttacagagcaagttctggatacacaggactacacatagaagaaacggtctccaaaataccaaaacaccaaatcagtagttggtgttggtgctgactatgtcactgggtgaaaggagcctagggggagggggagccacaagaaggaagtggaatcagggagtgtgtgtgtgtgtgtgtgtgtgtgtgtgtgtgagagagagagagagagagagagagattcttggaagaccggaagtgacaagtgacagccatcttggggtgtgccactgtgtgaggaaagagagcaacgtgactggaagagtctaagggaagggtgttgggcgctgagtcagatccaagtcagcacaagccagggaatgacaactctcctacatgtcaggtgtgtggctgtccttgatcacagctttcttcccacagcctgacagagcaggggctggccaaggttggccccaagcctcctgcagcctgcagcctgcagcctgtttctgtaaataagtcccgctggctcacggccacactcgctgcttcctccacatactgtttggagccatgtactgtttctgcagcctcgtatgcagggccagcaatatcacctggtccttctggaaaacctgtgaagatgggtgtggtctgaacaggctgcctgtaatttgaacaggaggcagaggcaggagtatcatcaccacaagttcaaagccagcctgatctacatagcccccagccaagcagagctaagtatatggaaaacccctcagtccttcccactctctccaaaagcttgcttatcctttttgggggatcgtgtgctctttacaggaggaagtatttacacacacggaaggtggttacttcctaaatccacagaacttagttagtttagcatctgcacatggatctaagactgctttcttgatcatatcttgatcaggagtatgggaccccaaaagaacttgggggacacatagccttaccagatgagctaaaaagctgtatccgaggcctccatttctggtaaatcactgcgagttaagtgaatggtgaggaactctgtcctcagtcaccctgcctcctcccttcctggggttgagagagagagagagagagagagagagagagagagtgcacatgcatgagcgtatatgcatgcatgtgtcctcaggaagataagttacccaggcaagaaagaccggctgcctgagcctagcgagtcctgtgtttcatctcttcccatctgctggggggagcttgtgaattctctcctgcttcctctttctcaagcccgtttcctcctt

SEQ ID NO: 4 - DNA, 20 bp, Artificial Sequence
  Synthetic oligonucleotide
gatggcccagggcgcgaagg

SEQ ID NO: 5 - DNA, 20 bp, Artificial Sequence
  Synthetic oligonucleotide
gagatggcccagggcgcgaa

SEQ ID NO: 6 - DNA, 20 bp, Artificial Sequence
  Synthetic oligonucleotide
cgactccttcctccgctccg

SEQ ID NO: 7 - DNA, 20 bp, Artificial Sequence
  Synthetic oligonucleotide
gcctcaggctgctcctcggc

SEQ ID NO: 8 - DNA, 20 bp, Artificial Sequence
  Synthetic oligonucleotide
ggcctcaggctgctcctcgg

SEQ ID NO: 9 - DNA, 20 bp, Artificial Sequence
  Synthetic oligonucleotide
ctcgtccctcctcccgctcc

SEQ ID NO: 10 - DNA, 20 bp, Artificial Sequence
  Synthetic oligonucleotide
caacggaaaagtctcaaaag

SEQ ID NO: 11 - DNA, 20 bp, Artificial Sequence
  Synthetic oligonucleotide
ggcaacggaaaagtctcaaa

SEQ ID NO: 12 - DNA, 20 bp, Artificial Sequence
  Synthetic oligonucleotide
gcggcaacggaaaagtctca

SEQ ID NO: 13 - DNA, 20 bp, Artificial Sequence
  Synthetic oligonucleotide
cagcggcaacggaaaagtct

SEQ ID NO: 14 - DNA, 20 bp, Artificial Sequence
  Synthetic oligonucleotide
cccagcggcaacggaaaagt

SEQ ID NO: 15 - DNA, 20 bp, Artificial Sequence
  Synthetic oligonucleotide
ctcccagcggcaacggaaaa

SEQ ID NO: 16 - DNA, 20 bp, Artificial Sequence
  Synthetic oligonucleotide
ggctcccagcggcaacggaa

SEQ ID NO: 17 - DNA, 20 bp, Artificial Sequence
  Synthetic oligonucleotide
ccggctcccagcggcaacgg

SEQ ID NO: 18 - DNA, 20 bp, Artificial Sequence
  Synthetic oligonucleotide
ctccggctcccagcggcaac

SEQ ID NO: 19 - DNA, 20 bp, Artificial Sequence
  Synthetic oligonucleotide
gcctccggctcccagcggca

SEQ ID NO: 20 - DNA, 20 bp, Artificial Sequence
  Synthetic oligonucleotide
gcgcctccggctcccagcgg

SEQ ID NO: 21 - DNA, 20 bp, Artificial Sequence
  Synthetic oligonucleotide
ccgcgcctccggctcccagc

SEQ ID NO: 22 - DNA, 20 bp, Artificial Sequence
  Synthetic oligonucleotide
ccccgcgcctccggctccca

SEQ ID NO: 23 - DNA, 20 bp, Artificial Sequence
  Synthetic oligonucleotide
gtccccgcgcctccggctcc

SEQ ID NO: 24 - DNA, 20 bp, Artificial Sequence
  Synthetic oligonucleotide
aggtccccgcgcctccggct

SEQ ID NO: 25 - DNA, 20 bp, Artificial Sequence
  Synthetic oligonucleotide
aagtcctgcctcctcgcggg

SEQ ID NO: 26 - DNA, 20 bp, Artificial Sequence
  Synthetic oligonucleotide
ggcaaagggaggcggtctgg

SEQ ID NO: 27 - DNA, 20 bp, Artificial Sequence
  Synthetic oligonucleotide
gcggcaaagggaggcggtct

SEQ ID NO: 28 - DNA, 20 bp, Artificial Sequence
  Synthetic oligonucleotide
cggcggcaaagggaggcggt

SEQ ID NO: 29 - DNA, 20 bp, Artificial Sequence
  Synthetic oligonucleotide
cccggcggcaaagggaggcg

SEQ ID NO: 30 - DNA, 20 bp, Artificial Sequence
  Synthetic oligonucleotide
tccccggcggcaaagggagg

SEQ ID NO: 31 - DNA, 20 bp, Artificial Sequence
  Synthetic oligonucleotide
cgtccccggcggcaaaggga

SEQ ID NO: 32 - DNA, 20 bp, Artificial Sequence
  Synthetic oligonucleotide
cccgagggctggtccggaat

SEQ ID NO: 33 - DNA, 20 bp, Artificial Sequence
  Synthetic oligonucleotide
aagtctttgcgggaggccgg

SEQ ID NO: 34 - DNA, 20 bp, Artificial Sequence
  Synthetic oligonucleotide
aaaagtctttgcgggaggcc

SEQ ID NO: 35 - DNA, 20 bp, Artificial Sequence
  Synthetic oligonucleotide
ggaaaagtctttgcgggagg

SEQ ID NO: 36 - DNA, 20 bp, Artificial Sequence
  Synthetic oligonucleotide
ggctcaggagacaggccggg

SEQ ID NO: 37 - DNA, 20 bp, Artificial Sequence
  Synthetic oligonucleotide
aagggtctaggatgcgcggg

SEQ ID NO: 38 - DNA, 20 bp, Artificial Sequence
  Synthetic oligonucleotide
caggtcggagagagatccgt

SEQ ID NO: 39 - DNA, 20 bp, Artificial Sequence
  Synthetic oligonucleotide
ggtgggtggtcttgaatagg

SEQ ID NO: 40 - DNA, 20 bp, Artificial Sequence
  Synthetic oligonucleotide
aaggtgggtggtcttgaata

SEQ ID NO: 41 - DNA, 20 bp, Artificial Sequence
  Synthetic oligonucleotide
agaaggtgggtggtcttgaa

SEQ ID NO: 42 - DNA, 20 bp, Artificial Sequence
  Synthetic oligonucleotide
ccagaaggtgggtggtcttg

SEQ ID NO: 43 - DNA, 20 bp, Artificial Sequence
  Synthetic oligonucleotide
taccagaaggtgggtggtct

SEQ ID NO: 44 - DNA, 20 bp, Artificial Sequence
  Synthetic oligonucleotide
ggtaccagaaggtgggtggt

SEQ ID NO: 45 - DNA, 20 bp, Artificial Sequence
  Synthetic oligonucleotide
ctggtaccagaaggtgggtg

SEQ ID NO: 46 - DNA, 20 bp, Artificial Sequence
  Synthetic oligonucleotide
atctggtaccagaaggtggg

SEQ ID NO: 47 - DNA, 20 bp, Artificial Sequence
  Synthetic oligonucleotide
cgatctggtaccagaaggtg

SEQ ID NO: 48 - DNA, 20 bp, Artificial Sequence
  Synthetic oligonucleotide
cgcgatctggtaccagaagg

SEQ ID NO: 49 - DNA, 20 bp, Artificial Sequence
  Synthetic oligonucleotide
ggcgcgatctggtaccagaa

SEQ ID NO: 50 - DNA, 20 bp, Artificial Sequence
  Synthetic oligonucleotide
tgggcgcgatctggtaccag

SEQ ID NO: 51 - DNA, 20 bp, Artificial Sequence
  Synthetic oligonucleotide
gatgggcgcgatctggtacc

SEQ ID NO: 52 - DNA, 20 bp, Artificial Sequence
  Synthetic oligonucleotide
cctagatgggcgcgatctgg

SEQ ID NO: 53 - DNA, 20 bp, Artificial Sequence
  Synthetic oligonucleotide
aacctagatgggcgcgatct

SEQ ID NO: 54 - DNA, 20 bp, Artificial Sequence
  Synthetic oligonucleotide
ataacctagatgggcgcgat

SEQ ID NO: 55 - DNA, 20 bp, Artificial Sequence
  Synthetic oligonucleotide
aaataacctagatgggcgcg

SEQ ID NO: 56 - DNA, 20 bp, Artificial Sequence
  Synthetic oligonucleotide
ggaaataacctagatgggcg

SEQ ID NO: 57 - DNA, 20 bp, Artificial Sequence
  Synthetic oligonucleotide
ggaggccccgcccctgcagg

SEQ ID NO: 58 - DNA, 20 bp, Artificial Sequence
  Synthetic oligonucleotide
gggctccggttctgcactct

SEQ ID NO: 59 - DNA, 20 bp, Artificial Sequence
  Synthetic oligonucleotide
tcgggctccggttctgcact

SEQ ID NO: 60 - DNA, 20 bp, Artificial Sequence
  Synthetic oligonucleotide
gctcgggctccggttctgca

SEQ ID NO: 61 - DNA, 20 bp, Artificial Sequence
  Synthetic oligonucleotide
aggctcgggctccggttctg

SEQ ID NO: 62 - DNA, 20 bp, Artificial Sequence
  Synthetic oligonucleotide
cctcaggctcgggctccggt

SEQ ID NO: 63 - DNA, 20 bp, Artificial Sequence
  Synthetic oligonucleotide
ggcctcaggctcgggctccg

SEQ ID NO: 64 - DNA, 20 bp, Artificial Sequence
  Synthetic oligonucleotide
ccattagcacgcgggtgacc

SEQ ID NO: 65 - DNA, 20 bp, Artificial Sequence
  Synthetic oligonucleotide
gagctctgatgtgttgaaga

SEQ ID NO: 66 - DNA, 20 bp, Artificial Sequence
  Synthetic oligonucleotide
ctaaggcgaaagccctcaat

SEQ ID NO: 67 - DNA, 20 bp, Artificial Sequence
  Synthetic oligonucleotide
atgtccacttgcagtgtgtt

SEQ ID NO: 68 - DNA, 20 bp, Artificial Sequence
  Synthetic oligonucleotide
gggttatgctggttgtacag

SEQ ID NO: 69 - DNA, 20 bp, Artificial Sequence
  Synthetic oligonucleotide
ctccaccttgggcttgcggc

SEQ ID NO: 70 - DNA, 20 bp, Artificial Sequence
  Synthetic oligonucleotide
ccttaaatacagcccccatg

SEQ ID NO: 71 - DNA, 20 bp, Artificial Sequence
  Synthetic oligonucleotide
gtccttaaatacagccccca

SEQ ID NO: 72 - DNA, 20 bp, Artificial Sequence
  Synthetic oligonucleotide
gtgtccttaaatacagcccc

SEQ ID NO: 73 - DNA, 20 bp, Artificial Sequence
  Synthetic oligonucleotide
gggtgtccttaaatacagcc

SEQ ID NO: 74 - DNA, 20 bp, Artificial Sequence
  Synthetic oligonucleotide
acgggtgtccttaaatacag

SEQ ID NO: 75 - DNA, 20 bp, Artificial Sequence
  Synthetic oligonucleotide
gcacgggtgtccttaaatac

SEQ ID NO: 76 - DNA, 20 bp, Artificial Sequence
  Synthetic oligonucleotide
ctctctccatctttaatggg

SEQ ID NO: 77 - DNA, 20 bp, Artificial Sequence
  Synthetic oligonucleotide
acagagatccgcagtcctct

SEQ ID NO: 78 - DNA, 20 bp, Artificial Sequence
  Synthetic oligonucleotide
cgcccaatgacacagagatc

SEQ ID NO: 79 - DNA, 20 bp, Artificial Sequence
  Synthetic oligonucleotide
ccttgatgccgggcaaagga

SEQ ID NO: 80 - DNA, 20 bp, Artificial Sequence
  Synthetic oligonucleotide
atctaactacagtagtgttc

SEQ ID NO: 81 - DNA, 20 bp, Artificial Sequence
  Synthetic oligonucleotide
tgtacagggcgagcacggcc

SEQ ID NO: 82 - DNA, 20 bp, Artificial Sequence
  Synthetic oligonucleotide
agccagtttcttctgccagt

SEQ ID NO: 83 - DNA, 20 bp, Artificial Sequence
  Synthetic oligonucleotide
gtgaaacaccgaggacacct

SEQ ID NO: 84 - DNA, 20 bp, Artificial Sequence
  Synthetic oligonucleotide
cctgccccttggtggaagcg

SEQ ID NO: 85 - DNA, 20 bp, Artificial Sequence
  Synthetic oligonucleotide
ggtttccccagccaccctga

SEQ ID NO: 86 - DNA, 20 bp, Artificial Sequence
  Synthetic oligonucleotide
ctgagtgggagccccgcccg

SEQ ID NO: 87 - DNA, 20 bp, Artificial Sequence
  Synthetic oligonucleotide
ttccccaaggctctgaacca

SEQ ID NO: 88 - DNA, 20 bp, Artificial Sequence
  Synthetic oligonucleotide
gtcagtgttaaaggaacctc

SEQ ID NO: 89 - DNA, 20 bp, Artificial Sequence
  Synthetic oligonucleotide
acacatgtgcatttgttggg

SEQ ID NO: 90 - DNA, 20 bp, Artificial Sequence
  Synthetic oligonucleotide
ttggcccggaggttactcag

SEQ ID NO: 91 - DNA, 20 bp, Artificial Sequence
  Synthetic oligonucleotide
tgaagttcattctgggtagg

SEQ ID NO: 92 - DNA, 20 bp, Artificial Sequence
  Synthetic oligonucleotide
attagttttccacccttaac

SEQ ID NO: 93 - DNA, 20 bp, Artificial Sequence
  Synthetic oligonucleotide
ttatacccgtttaatagatg

SEQ ID NO: 94 - DNA, 20 bp, Artificial Sequence
  Synthetic oligonucleotide
tacactggtcactcaatcat

SEQ ID NO: 95 - DNA, 20 bp, Artificial Sequence
  Synthetic oligonucleotide
aggtcaagccatgtggcacc

SEQ ID NO: 96 - DNA, 20 bp, Artificial Sequence
  Synthetic oligonucleotide
caagacagagtgactctaga

SEQ ID NO: 97 - DNA, 20 bp, Artificial Sequence
  Synthetic oligonucleotide
acagcaataacattaagctc

SEQ ID NO: 98 - DNA, 20 bp, Artificial Sequence
  Synthetic oligonucleotide
tgtgtgaccatgggcagtta

SEQ ID NO: 99 - DNA, 20 bp, Artificial Sequence
  Synthetic oligonucleotide
cccctaaaatgcagagtaag

SEQ ID NO: 100 - DNA, 20 bp, Artificial Sequence
  Synthetic oligonucleotide
aagtcgactaaggctggcac

SEQ ID NO: 101 - DNA, 20 bp, Artificial Sequence
  Synthetic oligonucleotide
tgtgaccttgaggaagtggt

SEQ ID NO: 102 - DNA, 20 bp, Artificial Sequence
  Synthetic oligonucleotide
aaatgaagggaggcgatcag

SEQ ID NO: 103 - DNA, 20 bp, Artificial Sequence
  Synthetic oligonucleotide
gtggaccttgtaaccagccg

SEQ ID NO: 104 - DNA, 20 bp, Artificial Sequence
  Synthetic oligonucleotide
tcctaggatgcaaagagtct

SEQ ID NO: 105 - DNA, 20 bp, Artificial Sequence
  Synthetic oligonucleotide
tctgcaacatccaaaatagt

SEQ ID NO: 106 - DNA, 20 bp, Artificial Sequence
  Synthetic oligonucleotide
ctatgagttaacattccctc

SEQ ID NO: 107 - DNA, 20 bp, Artificial Sequence
  Synthetic oligonucleotide
gactaatgttctataaaccc

SEQ ID NO: 108 - DNA, 20 bp, Artificial Sequence
  Synthetic oligonucleotide
tagaagtcatttctaatgat

SEQ ID NO: 109 - DNA, 20 bp, Artificial Sequence
  Synthetic oligonucleotide
gccgaaggtgttttcttgcc

SEQ ID NO: 110 - DNA, 20 bp, Artificial Sequence
  Synthetic oligonucleotide
cttccccaaacaggcttcca

SEQ ID NO: 111 - DNA, 20 bp, Artificial Sequence
  Synthetic oligonucleotide
aagtgaccccaggacaaaca

SEQ ID NO: 112 - DNA, 20 bp, Artificial Sequence
  Synthetic oligonucleotide
gattagccaatcactcaggt

SEQ ID NO: 113 - DNA, 20 bp, Artificial Sequence
  Synthetic oligonucleotide
gttccccagctacctagcca

SEQ ID NO: 114 - DNA, 20 bp, Artificial Sequence
  Synthetic oligonucleotide
tccaggcctttgcacaggct

SEQ ID NO: 115 - DNA, 20 bp, Artificial Sequence
  Synthetic oligonucleotide
tgggcaattattgaataaaa

SEQ ID NO: 116 - DNA, 20 bp, Artificial Sequence
  Synthetic oligonucleotide
gtcttggttatcactatgtc

SEQ ID NO: 117 - DNA, 20 bp, Artificial Sequence
  Synthetic oligonucleotide
ttgaccaagacagatgagct

SEQ ID NO: 118 - DNA, 20 bp, Artificial Sequence
  Synthetic oligonucleotide
gcttgggactcagcattgac

SEQ ID NO: 119 - DNA, 20 bp, Artificial Sequence
  Synthetic oligonucleotide
gagagggaagccagtctgag

SEQ ID NO: 120 - DNA, 20 bp, Artificial Sequence
  Synthetic oligonucleotide
aacctggagcacctggtcag

SEQ ID NO: 121 - DNA, 20 bp, Artificial Sequence
  Synthetic oligonucleotide
tcagcccaagcacagcagca

SEQ ID NO: 122 - DNA, 20 bp, Artificial Sequence
  Synthetic oligonucleotide
ctaaaggagacagatgctca

SEQ ID NO: 123 - DNA, 20 bp, Artificial Sequence
  Synthetic oligonucleotide
ttgaattccaacaatcacag

SEQ ID NO: 124 - DNA, 20 bp, Artificial Sequence
  Synthetic oligonucleotide
gtgaccttccaactttgaat

SEQ ID NO: 125 - DNA, 20 bp, Artificial Sequence
  Synthetic oligonucleotide
ttctagcattctagaatccc

SEQ ID NO: 126 - DNA, 20 bp, Artificial Sequence
  Synthetic oligonucleotide
aattctagcattctagaatc

SEQ ID NO: 127 - DNA, 20 bp, Artificial Sequence
  Synthetic oligonucleotide
gattccaatgtttcagcttt

SEQ ID NO: 128 - DNA, 20 bp, Artificial Sequence
  Synthetic oligonucleotide
ggtatccacaattggccagt

SEQ ID NO: 129 - DNA, 20 bp, Artificial Sequence
  Synthetic oligonucleotide
gagataccaatattctgctt

SEQ ID NO: 130 - DNA, 20 bp, Artificial Sequence
  Synthetic oligonucleotide
aacattccaacactgagttc

SEQ ID NO: 131 - DNA, 20 bp, Artificial Sequence
  Synthetic oligonucleotide
tcaagaggttcaaactgaca

SEQ ID NO: 132 - DNA, 20 bp, Artificial Sequence
  Synthetic oligonucleotide
aattccagtatgccagtatt

SEQ ID NO: 133 - DNA, 20 bp, Artificial Sequence
  Synthetic oligonucleotide
ccaacctttgaggatcttgg

SEQ ID NO: 134 - DNA, 20 bp, Artificial Sequence
  Synthetic oligonucleotide
gaatccaacatttcagcttt

SEQ ID NO: 135 - DNA, 20 bp, Artificial Sequence
  Synthetic oligonucleotide
aagggaggaataaggtcaga

SEQ ID NO: 136 - DNA, 20 bp, Artificial Sequence
  Synthetic oligonucleotide
gtaggctattaatagttaag

SEQ ID NO: 137 - DNA, 20 bp, Artificial Sequence
  Synthetic oligonucleotide
ttccactcaatgaatggaaa

SEQ ID NO: 138 - DNA, 20 bp, Artificial Sequence
  Synthetic oligonucleotide
gacagcaagaccaacacctt

SEQ ID NO: 139 - DNA, 20 bp, Artificial Sequence
  Synthetic oligonucleotide
tttgaactacatgggtcctc

SEQ ID NO: 140 - DNA, 20 bp, Artificial Sequence
  Synthetic oligonucleotide
attcaagtaaggtctacaca

SEQ ID NO: 141 - DNA, 20 bp, Artificial Sequence
  Synthetic oligonucleotide
aggattcaagtaaggtctac

SEQ ID NO: 142 - DNA, 20 bp, Artificial Sequence
  Synthetic oligonucleotide
gatatctagaggaatatcta

SEQ ID NO: 143 - DNA, 20 bp, Artificial Sequence
  Synthetic oligonucleotide
attccttgatatctagagga

SEQ ID NO: 144 - DNA, 20 bp, Artificial Sequence
  Synthetic oligonucleotide
ttcaaatgtatctctaatta

SEQ ID NO: 145 - DNA, 20 bp, Artificial Sequence
  Synthetic oligonucleotide
cacatgcaatccaccgtgtt

SEQ ID NO: 146 - DNA, 20 bp, Artificial Sequence
  Synthetic oligonucleotide
ggccaatttccattgcatct

SEQ ID NO: 147 - DNA, 20 bp, Artificial Sequence
  Synthetic oligonucleotide
gaacaaattttcctatgaaa

SEQ ID NO: 148 - DNA, 20 bp, Artificial Sequence
  Synthetic oligonucleotide
ttgaacaagccgtctaggtg

SEQ ID NO: 149 - DNA, 20 bp, Artificial Sequence
  Synthetic oligonucleotide
ggcagcatcacctgggaact

SEQ ID NO: 150 - DNA, 20 bp, Artificial Sequence
  Synthetic oligonucleotide
tctgggaaaaagagtcctgg

SEQ ID NO: 151 - DNA, 20 bp, Artificial Sequence
  Synthetic oligonucleotide
tttccaagagccacagaagc

SEQ ID NO: 152 - DNA, 20 bp, Artificial Sequence
  Synthetic oligonucleotide
ttttccataataaaggaatt

SEQ ID NO: 153 - DNA, 20 bp, Artificial Sequence
  Synthetic oligonucleotide
ctggatgagagtttacgggc

SEQ ID NO: 154 - DNA, 20 bp, Artificial Sequence
  Synthetic oligonucleotide
agtgcaatacggtattgcag

SEQ ID NO: 155 - DNA, 20 bp, Artificial Sequence
  Synthetic oligonucleotide
aatgcccaagtcctcaccgt

SEQ ID NO: 156 - DNA, 20 bp, Artificial Sequence
  Synthetic oligonucleotide
tgtgcaacaaatgtttattg

SEQ ID NO: 157 - DNA, 20 bp, Artificial Sequence
  Synthetic oligonucleotide
cacaccctggaacatacaaa

SEQ ID NO: 158 - DNA, 20 bp, Artificial Sequence
  Synthetic oligonucleotide
gcaatgcttaagacaagcct

SEQ ID NO: 159 - DNA, 20 bp, Artificial Sequence
  Synthetic oligonucleotide
tcactaacacagattaagca

SEQ ID NO: 160 - DNA, 19 bp, Artificial Sequence
  primer
ctctccgacctgccacaga

SEQ ID NO: 161 - DNA, 28 bp, Artificial Sequence
  probe
ccctattcaagaccacccaccttctggt

SEQ ID NO: 162 - DNA, 18 bp, Artificial Sequence
  primer
aaacggaagcgcatcgaa

SEQ ID NO: 163 - DNA, 20 bp, Artificial Sequence
  primer
gggactggcgagccttagtt

SEQ ID NO: 164 - DNA, 23 bp, Artificial Sequence
  probe
ccatccgtggccagatcctgtcc

SEQ ID NO: 165 - DNA, 20 bp, Artificial Sequence
  Synthetic oligonucleotide
gtccaccattagcacgcggg

SEQ ID NO: 166 - DNA, 20 bp, Artificial Sequence
  Synthetic oligonucleotide
gtccttaaatacagccccgg